(19) United States
(12) Patent Application Publication
(10) Pub. No.: US 2024/0408193 A1
(43) Pub. Date: Dec. 12, 2024

(54) SARS-COV-2 VACCINES

(71) Applicants: NEC ONCOIMMUNITY AS, Oslo (NO); NEC LABORATORIES EUROPE GMBH, Heidelberg (DE)

(72) Inventors: Richard Stratford, Oslo (NO); Trevor Clancy, Oslo (NO); Clément Moliné, Oslo (NO); Boris Simovski, Oslo (NO); Brandon Malone, Heidelberg (DE); Jun Cheng, Heidelberg (DE)

(21) Appl. No.: 17/996,608

(22) PCT Filed: Apr. 20, 2021
(86) PCT No.: PCT/EP2021/060272

§ 371 Date: Oct. 19, 2022

(30) Foreign Application Priority Data
Apr. 20, 2020 (EP) 20170488.9
Jul. 24, 2020 (EP) 20187750.3

(51) Int. Cl. A61K 39/215; G01N 33/569

(52) U.S. Cl. CPC A61K 39/215; G01N 33/56983; G01N 2333/165

(57) ABSTRACT

The present invention relates to a coronavirus vaccine composition, comprising one or more epitopes suitable for stimulating a broad adaptive immune response across a plurality of human leukocyte antigen (HLA) populations, for either MHC Class I and/or MHC Class II immunogenicity. The selection of such epitopes is made possible by the generation of predictive data by an artificial intelligence (AI)-driven platform, through the analysis of large scale epitope mapping of the SARS-CoV-2 proteome and epitope scoring based upon predicted immunogenicity, followed by robust statistical analysis and Monte Carlo-based simulation. The vaccine compositions of the present invention are suitable for use in the therapeutic or prophylactic treatment of SARS-CoV-2 infections. The invention also describes methods for using said compositions.

### FIELD OF INVENTION

The present invention relates to vaccine compositions optimised for the prophylactic or therapeutic treatment of an infection caused by SARS-CoV-2, wherein said vaccine compositions are comprised of one or more epitopes selected for their ability to stimulate a broad and effective adaptive immune response across a diverse spectrum of human leukocyte antigen (HLA) populations.

### BACKGROUND

The outbreak of coronavirus disease 2019 (COVID-19) and its rapid worldwide transmission resulted in its declaration as a pandemic and global health emergency by the World Health Organisation (WHO). COVID-19 is caused by Severe Acute Respiratory Syndrome Coronavirus 2 (SARS-CoV-2), a positive-sense RNA coronavirus that has an envelope encapsulating its large RNA genome and is further characterised by an exposed spike glycoprotein (S-protein), projecting from its viral surface (Gorbalenya et al. 2020, Nat Microbiol (4): 536-544).

Whilst the majority of COVID-19 cases result only in mild symptoms including fever, cough, or shortness of breath, a significant number of cases progress to viral pneumonia and multi-organ failure (Hui et al. 2020, Int J Infect Dis 91:264-66). The rapid rise in the number of infections and deaths around the globe highlights the urgent need for better therapeutic and prophylactic interventions to combat the disease and an effective vaccine has been hailed by many as a crucial cornerstone in our potential fight against the SARS-CoV-2 virus.

Vaccination has been established as an effective form of epidemiological control, and vaccines have had significant success in aiding the decline of infections and mortalities associated with viral infections such as smallpox and polio. Other infections, however, have proven harder to vaccinate against. Much of the global efforts to develop Coronaviridae vaccines to date have focused primarily on stimulating an antibody response against the S-protein, serving as the most exposed structural protein on the virus.

However, although responses against the S-protein of closely-related SARS-CoV have been shown to confer short-term protection in mice (Yang et al. 2004, Nature 428 (6982): 561-4), neutralising antibody responses against the same structure in convalescent patients are typically of low titre and short-lived (Channappanavar et al. 2014, Immunol Res 88 (19): 11034-44) (Yang et al. 2006, Clin Immunol 120 (2) 171-8). Furthermore, the induction of antibody responses to S-protein in SARS-CoV has been associated with harmful effects in some animal models, raising possible safety concerns regarding the use of the S-protein as a vaccine target. In macaque models, for example, it was observed that anti-S-protein antibodies were associated with severe acute lung injury (Liu et al. 2019 JCI Insight 4 (4)), whilst sera from SARS-CoV patients also revealed that elevated anti-S-protein antibodies were observed in those patients that succumbed to the disease.

Further concerns over an S-protein-centred approach arise when considering the possibility of antibody-dependent enhancement (ADE), a biological phenomenon wherein antibodies facilitate viral entry into host cells and enhance the infectivity of the virus (Tirado & Yoon 2003, Viral Immunol 16 (1) 69-86). It has been demonstrated that a neutralising antibody may bind to the S-protein of a Coronavirus, triggering a conformational change that facilitates viral entry (Wan et al. J Virol 2020, 94 (5)). As such, there is increasing evidence to suggest that a vaccine designed to generate anti-S-protein antibodies via a humoral immune response may not, in fact, offer an effective and safe method of providing protection against SARS-CoV-2 infection.

As an alternative arm of the adaptive immune system that is also specialised to resolve infections and prevent reinfection from pathogens, cellular immunity often works in tandem with humoral-antibody-based-immunity upon natural exposure to a foreign body. A cellular immune response involves the interaction of T cells, each providing a variety of immune-related functions to aid in the reduction or elimination of pathogen-infected host cells (Amanna & Slifka 2011, Virology 411 (2): 206-215). Furthermore, the generation of memory T cells as part of the cellular immune response results in the ability to mount a faster and stronger immune response upon re-exposure to a previously encountered pathogen (Restifo & Tattinoni 2013, Current Opinion in Immunology 25 (5): 556-63). As SARS-CoV-2 vaccine development has been focused on activating a neutralising antibody-based humoral immune response, most commonly through the generation of S protein-based subunit vaccines (Amanat & Krammer 2020, Cell Press Immunity 52:583-589), however such subunit vaccines are unlikely to generate robust cellular immune responses in a broad population (Testa & Philip 2012, Future Virol 7 (11): 1077-1088).

However, when designing vaccines engineered to instigate a broad T cell response, there exists a further challenge of human leukocyte antigen (HLA) restriction within an individual and a broader population. An HLA system is a gene complex encoding the major histocompatibility complex (MHC) proteins in humans, responsible for the regulation of an individual's immune system, as well as the ability to specifically present at the surface of infected cells, and elicit an immune response against, epitopes delivered to said individual in the form of a vaccine (Marsh et al. 2010 Tissue Antigens 75 (4): 291-455).

The high polymorphism of HLA alleles and subsequent immune system variability between individuals results in a diverse spectrum of “HLA types” across the population. As an added complication to peptide-based vaccine development, such HLA types can have a significant impact on the efficacy of a potentially prophylactic viral vaccine composition between different individuals. As such, generation of an epitope-based vaccine composition that is compatible with a particular subset of HLA types may prove ineffective with a significant proportion of the global population comprising individuals with different HLA types. In light of this, the generation of T-cell and B-cell epitope vaccines, that target a limited number of HLA types, may only prove advantageous for a narrow, select population.

The current lack of an approved vaccine composition which is efficacious across a wide range of HLA populations, creates significant danger for at-risk populations, including health care workers and patients in acute danger of nosocomial or community-transmitted infections.

Thus, there exists an urgent need for a safe and effective vaccine for use in the therapeutic or prophylactic treatment of COVID-19, optimised to incorporate epitopes covering a diverse spectrum of HLA types, with the potential to stimulate a broad adaptive immune response against SARS-CoV-2 across the global human population.

### SUMMARY OF INVENTION

This invention is based on the surprising discovery that, by using an extensive artificial intelligence (AI) platform to identify predicted SARS-CoV-2 epitopes that bind HLA molecules across a broad spectrum of HLA types, a safe and effective vaccine can be formulated that comprises one or more of said epitopes. Such a vaccine thus has the potential to stimulate a broad adaptive immune response to SARS-CoV-2 that is both cellular and humoral in nature, for the therapeutic or prophylactic treatment of COVID-19 in humans across the global population.

In a first aspect of the invention, there is provided a coronavirus vaccine composition, comprising one or more epitopes found within any one or more hotspot regions identified in FIGS. 1-10, or a polynucleotide encoding said epitope, wherein each epitope is at least 8 amino acids in length, and wherein each epitope has a mean antigen presentation (AP) cut off value according to the following table:

| | Mean Antigen |
|---|---|
| | Presentation |
| | (AP) |
| | Cut-Off |
| | Value |
| Averaged HLA Type of MHC Class I | ≥0.4 |
| Averaged HLA Type of MHC Class II | ≤13 |

or a mean immune presentation (IP) score of at least 0.5, and wherein an antigen presentation (AP) value or immune presentation value is a prediction score assigned to each amino acid as shown in FIGS. 1-10 for each hotspot region, and wherein the mean AP cut-off value is the value, averaged across all amino acids within an epitope, for which said epitope is considered able to stimulate a broad adaptive immune response across a plurality of HLA types, for either MHC Class I and/or MHC Class II immunogenicity.

In a second aspect of the invention, there is provided a coronavirus vaccine composition, comprising an immunogenic portion of the coronavirus, said immunogenic portion consisting of one or more epitopes found within any one or more hotspot regions identified in FIGS. 1-10, or a polynucleotide encoding said epitope, wherein each of said epitope is at least 8 amino acids in length, and wherein each of said epitope is considered able to stimulate a broad adaptive immune response across a plurality of HLA types, for either MHC Class I and/or MHC Class II immunogenicity.

In a third aspect of the invention, there is provided a coronavirus vaccine composition, comprising one or more epitopes found within Table 1, or a polynucleotide encoding said epitope, wherein each epitope is at least 8 amino acids in length, preferably 9 amino acids, and wherein the epitope is considered able to stimulate a broad adaptive immune response across a plurality of HLA types, for either MHC Class I immunogenicity, optionally wherein said composition also further comprises any of the one or more epitopes according to first or second aspects of the invention.

In a fourth aspect of the invention, there is provided a coronavirus vaccine composition according to the first, second or third aspects of the invention, for use in the therapeutic or prophylactic treatment of a coronavirus infection in a subject.

In a fifth aspect of the invention, there is provided a use of a coronavirus vaccine composition according to the first, second or third aspects of the invention, in the manufacture of a medicament for the therapeutic or prophylactic treatment of a coronavirus infection.

In a sixth aspect of the invention, there is provided a diagnostic assay to determine whether a patient has or has had prior infection with SARS-CoV-2, wherein the diagnostic assay is carried out on a biological sample obtained from a subject, and wherein the diagnostic assay comprises the utilisation or identification within the biological sample of one or more epitopes according to any of the appended claims.

### BRIEF DESCRIPTION OF FIGURES

FIG. 1 shows a full amino acid sequence of SARS-CoV-2 ORF1ab, wherein each amino acid has been given two antigen presentation (AP) scores and an immune presentation (IP) score. The first two columns of “AA” and “SEQ” relate to the amino acid number and amino acid type, respectively. The first AP score (labelled MHC I) is the antigen presentation value for a chosen amino acid, averaged across 66 HLA alleles that correspond to MHC Class I, whilst the second AP score (labelled MHC II) is the antigen presentation value for the same chosen amino acid, averaged across 34 HLA alleles that correspond to MHC Class II. Regions that contain epitopes that satisfy the desired IP score found within ORF1ab are also highlighted in grey within this figure.

FIG. 2 shows a full amino acid sequence of SARS-CoV-2 spike(S) protein, wherein each amino acid has been given two antigen presentation (AP) scores and an IP score akin to FIG. 1. Regions that contain epitopes that satisfy the desired IP score found within the S protein are also notated within this figure.

FIG. 3 shows a full amino acid sequence of SARS-CoV-2 ORF3a, wherein each amino acid has been given two antigen presentation (AP) scores and an IP score akin to FIG. 1. Regions that contain epitopes that satisfy the desired IP score found within ORF3a are also notated within this figure.

FIG. 4 shows a full amino acid sequence of SARS-CoV-2 envelope (E) protein, wherein each amino acid has been given two antigen presentation (AP) scores and an IP score akin to FIG. 1. Regions that contain epitopes that satisfy the desired IP score found within the E protein are also notated within this figure.

FIG. 5 shows a full amino acid sequence of SARS-CoV-2 membrane (M) protein, wherein each amino acid has been given two antigen presentation (AP) scores and an IP score akin to FIG. 1. Regions that contain epitopes that satisfy the desired IP score found within the M protein are also notated within this figure.

FIG. 6 shows a full amino acid sequence of SARS-CoV-2 ORF6, wherein each amino acid has been given two antigen presentation (AP) scores and an IP score akin to FIG. 1. Regions that contain epitopes that satisfy the desired IPAP score found within ORF6 are also notated within this figure.

FIG. 7 shows a full amino acid sequence of SARS-CoV-2 ORF7a, wherein each amino acid has been given two antigen presentation (AP) scores and an IP score akin to FIG. 1. Regions that contain epitopes that satisfy the desired IP score found within ORF7a are also notated within this figure.

FIG. 8 shows a full amino acid sequence of SARS-CoV-2 ORF8, wherein each amino acid has been given two antigen presentation (AP) scores and an IP score akin to FIG. 1. Regions that contain epitopes that satisfy the desired IP score found within ORF8 are also notated within this figure.

FIG. 9 shows a full amino acid sequence of SARS-CoV-2 nucleocapsid (N) protein, wherein each amino acid has been given two antigen presentation (AP) scores and an IP score akin to FIG. 1. Regions that contain epitopes that satisfy the desired IP score found within the N protein are also notated within this figure.

FIG. 10 shows a full amino acid sequence of SARS-CoV-2 ORF10, wherein each amino acid has been given two antigen presentation (AP) scores and an IP score akin to FIG. 1. Regions that contain epitopes that satisfy the desired IP score found within ORF10 are also highlighted within this figure.

FIG. 11 shows the top 100 HLA-A and HLA-B Class I alleles and HLA-DR Class II alleles used for analysis according to the present invention.

FIG. 12 shows a schematic of the weighted bipartite graph matching problem setting according to Example 5.

FIG. 13 shows a table of defined unfiltered hotspots from any of FIGS. 1-10, each of which meet the required AP scores.

FIG. 14 shows a table of defined unfiltered hotspots from any of FIGS. 1-10, each of which meet the required IP scores.

FIG. 15 shows a table of filtered hotspots from any of FIGS. 1-10, each of which meet the required AP scores.

FIG. 16 shows a table of filtered hotspots from any of FIGS. 1-10, each of which meet the required IP scores.

FIG. 17 shows a table of hotspots selected following digital twin analysis, each meeting the required AP scores, representing a preferred selection of hotspots.

FIG. 18 shows a table of hotspots selected following digital twin analysis, each meeting the required IP scores, representing a further preferred selection of hotspots.

FIG. 19 shows a selection of preferred epitopes, wherein said epitopes may overlap with more than one hotspot.

FIG. 20 shows the peptides selected in Example 6 for a patient study.

FIG. 21 shows the ELISpot assay results for IFNγ response in seven patients tested with allele-specific peptide pools.

FIG. 22 shows a heatmap of 10 patients tested with pan-allele peptide pools.

FIGS. 23 to 34 show (a) violin plots for each hotspot region with patient results for both (i) IFNγ secretion response and (ii) T-cell proliferation response after restimulation with predicted peptides, and (b) heatmaps for each hotspot region with patient results for both (i) IFNγ secretion response and (ii) T-cell proliferation response after restimulation with predicted peptides.

FIG. 35 shows hotspot immunogenicity as measured by (a) IFNγ-secretion and (b) T cell proliferation (3H-thymidine CPM count).

FIG. 36 shows the number of hotspots recognised per donor as measured by (a) IFNγ-secretion and (b) T cell proliferation (3H-thymidine CPM count).

FIG. 37 shows the 67 peptides and the hotspot regions that were validated in Example 7.

### DETAILED DESCRIPTION OF THE INVENTION

This invention is predicated on the development of an artificial intelligence (AI) platform that can predict SARS-CoV-2 epitopes that would safely and most effectively stimulate a broad adaptive immune response to SARS-CoV-2 that is both cellular and humoral in nature, and that the incorporation of such epitopes into a vaccine composition would allow for the therapeutic or prophylactic treatment of coronavirus disease 19 (COVID-19). It is envisaged that the vaccine composition of the present invention may differ from other COVID-19 vaccination approaches through its design to stimulate a broad adaptive immune response through the specific activation of CD8+ and CD4+ T cells, aiming to generate a more substantial level of immunity. Furthermore, a surprisingly robust statistical model allows for the identification of those predicted SARS-CoV-2 epitopes that are capable of triggering immunogenicity across a wide variety of human leukocyte antigen (HLA) types, hence the vaccine composition may have the potential to elicit protection against the coronavirus across the global human population.

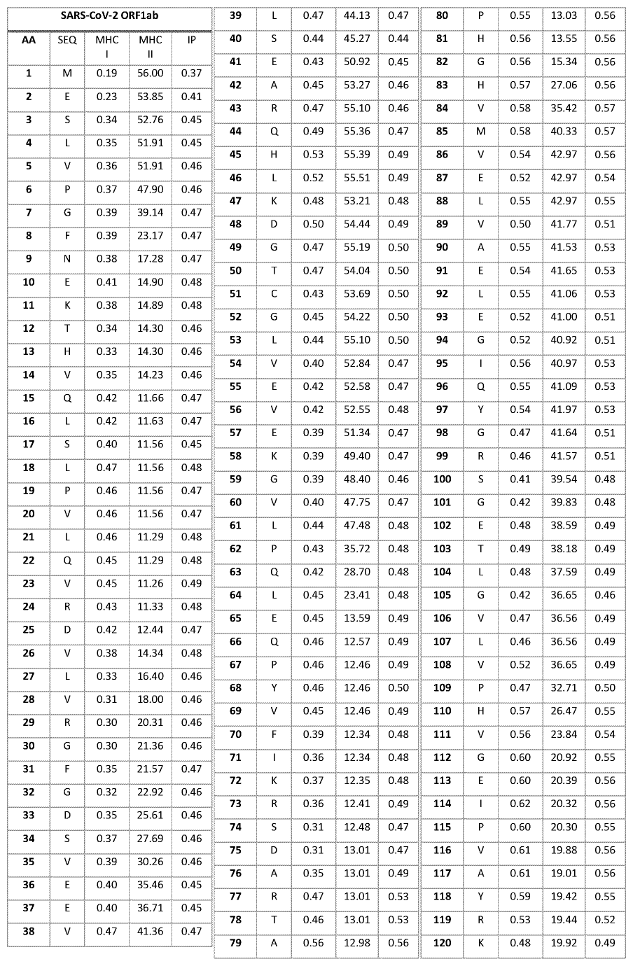

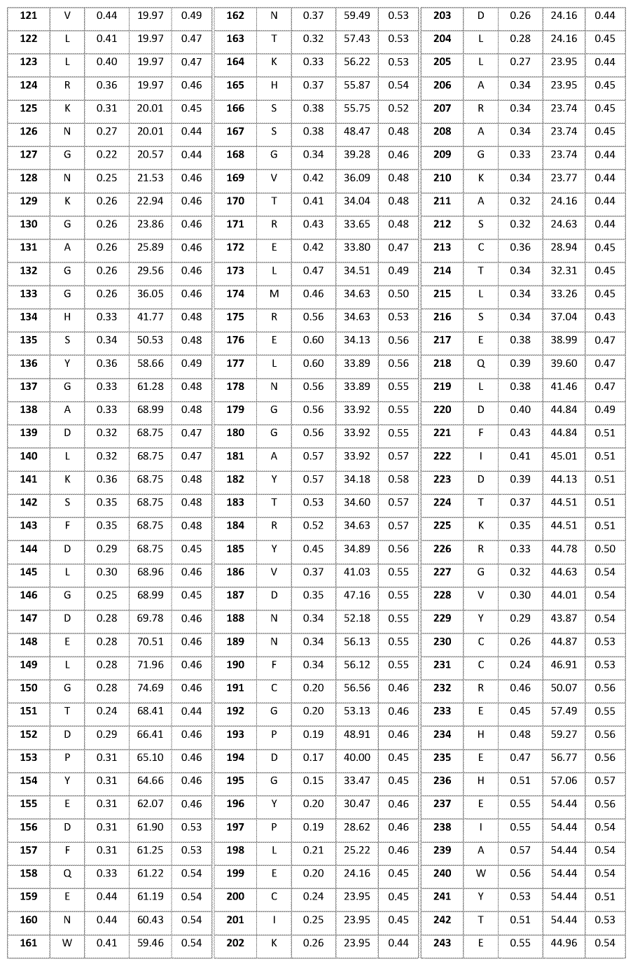

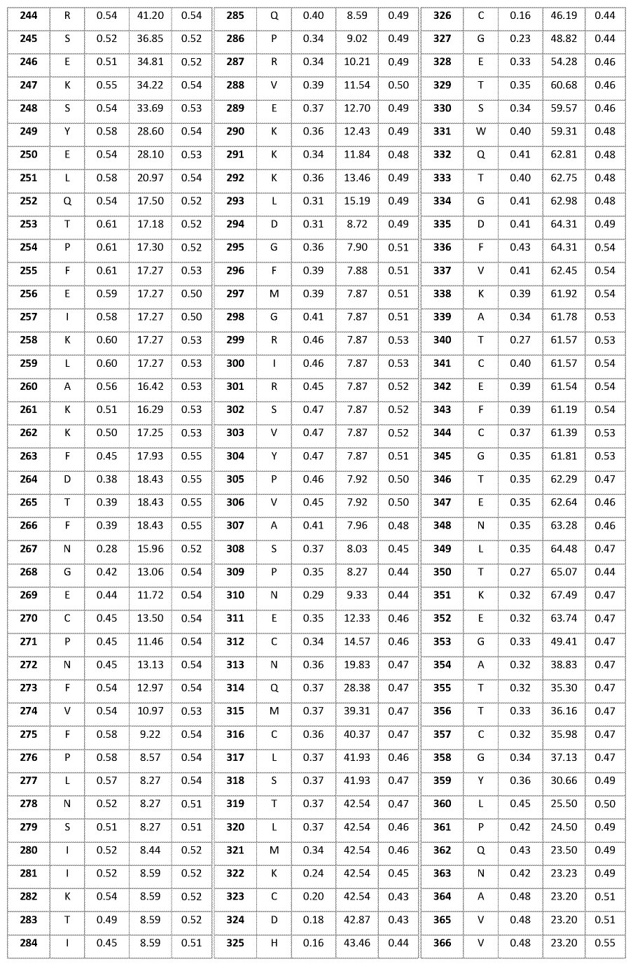

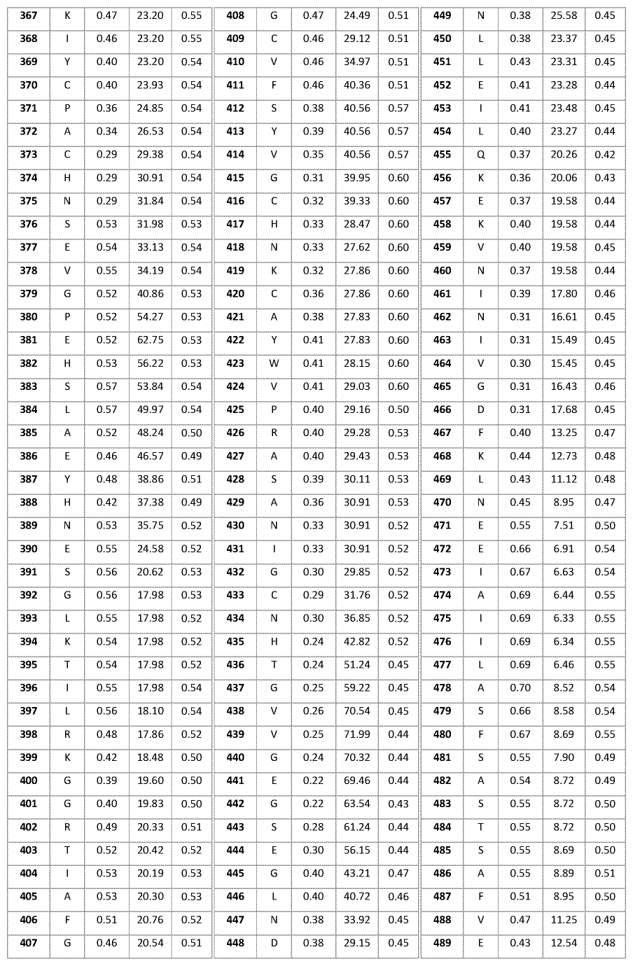

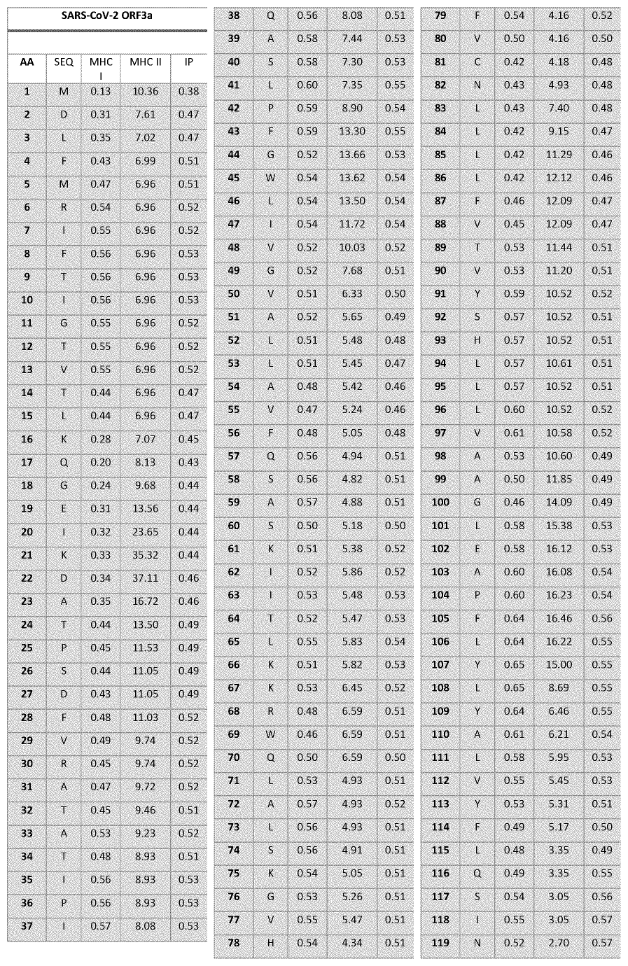

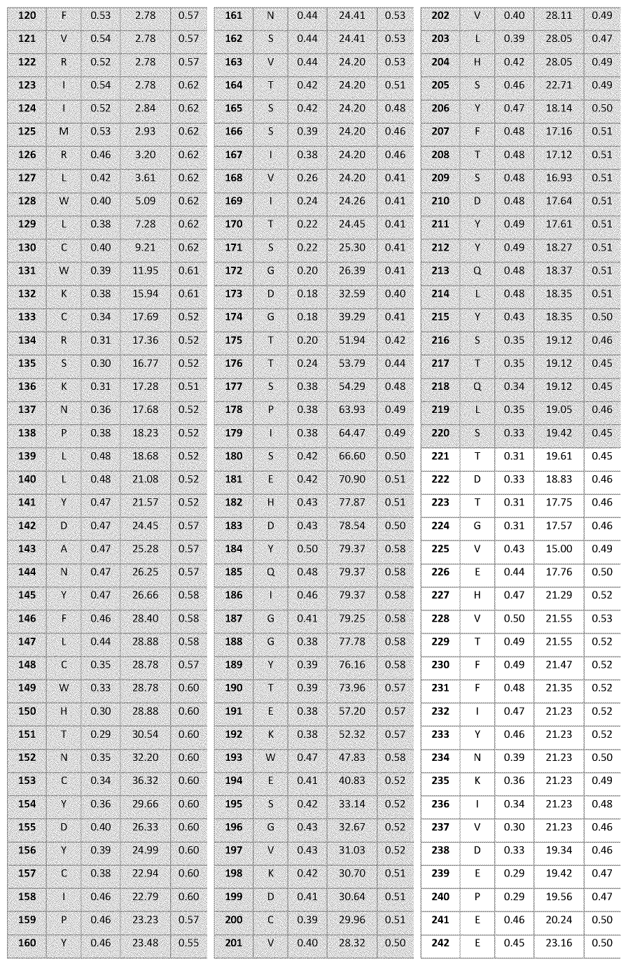

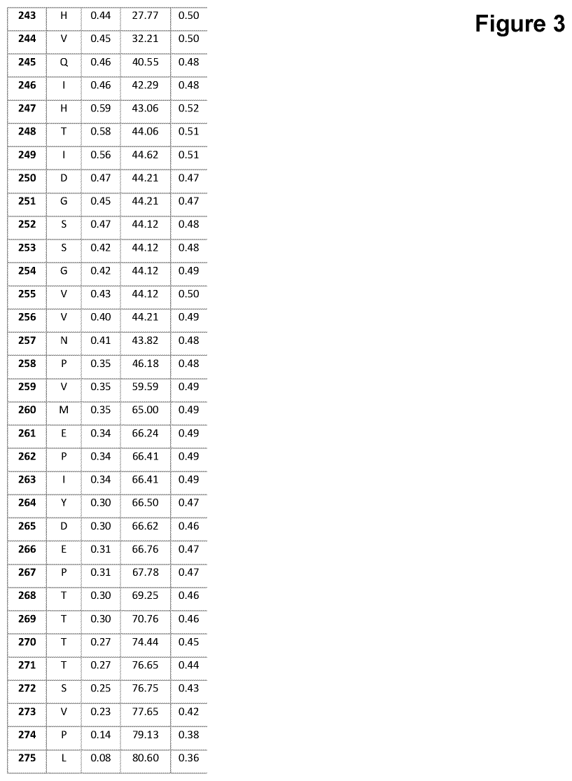

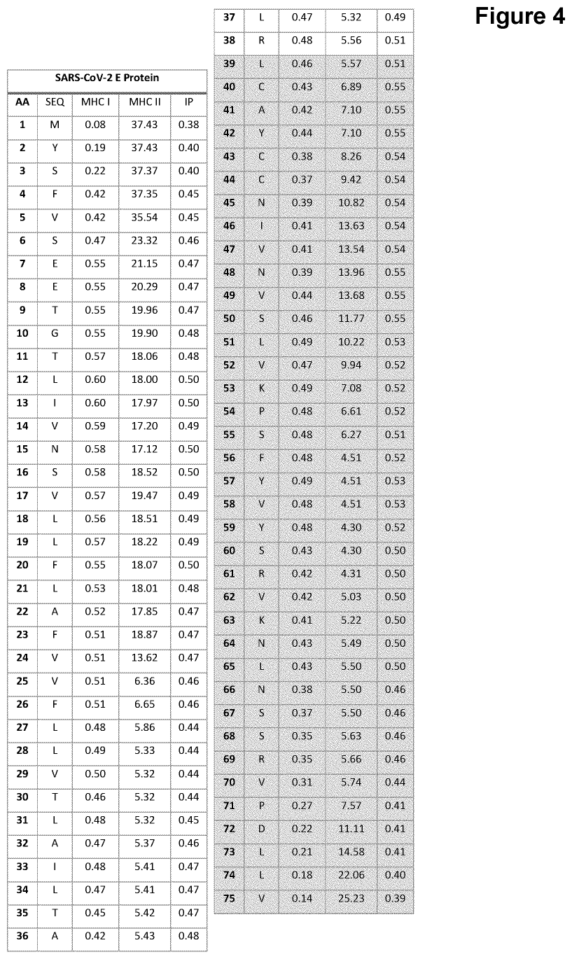

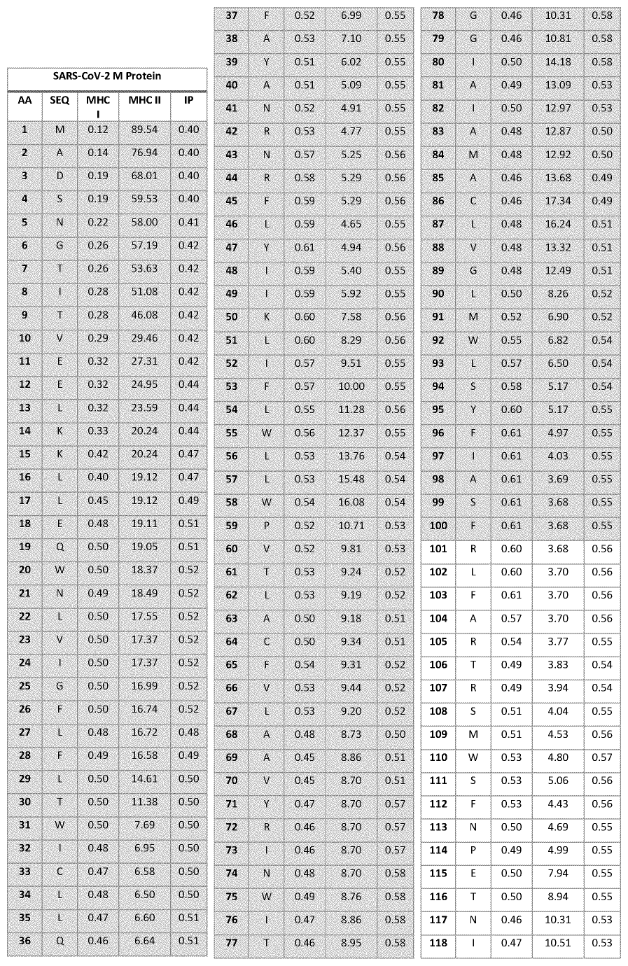

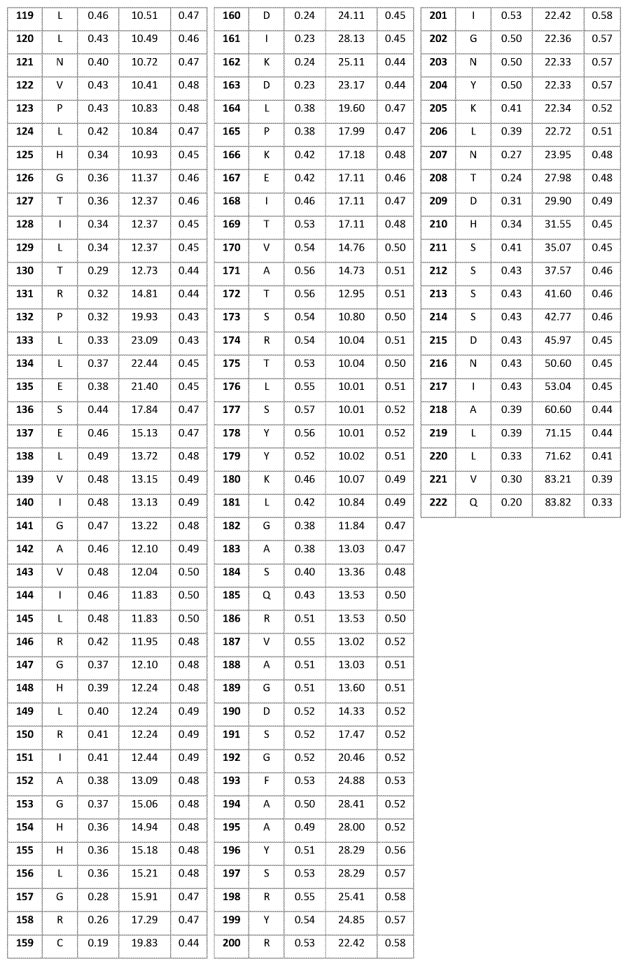

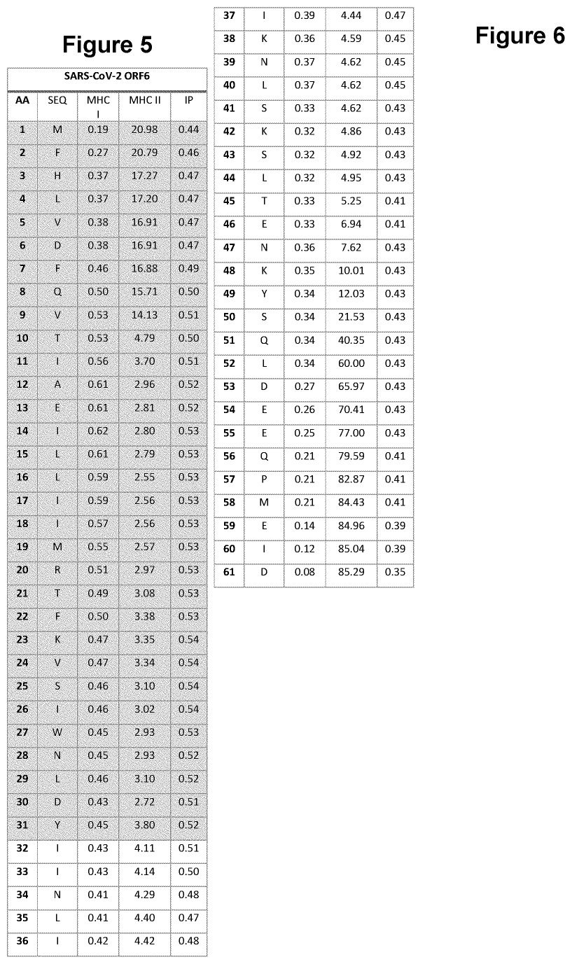

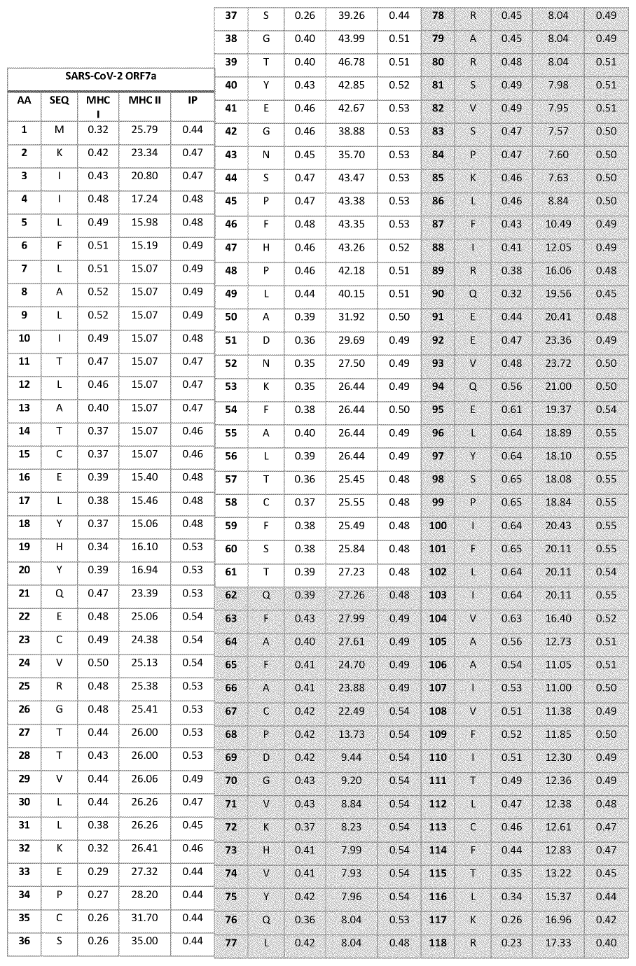

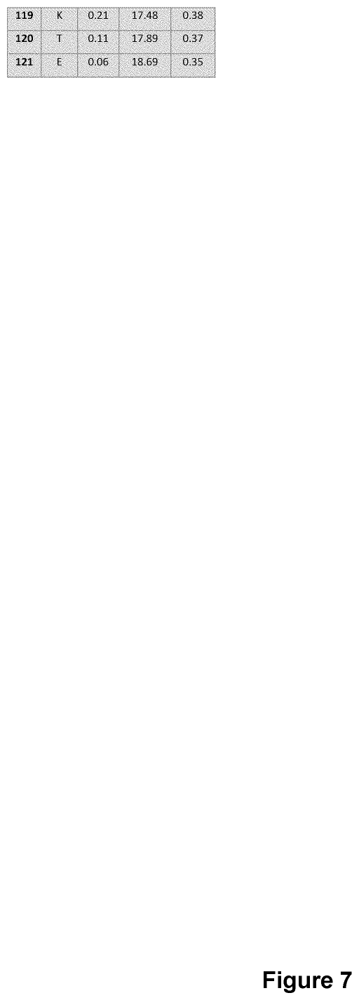

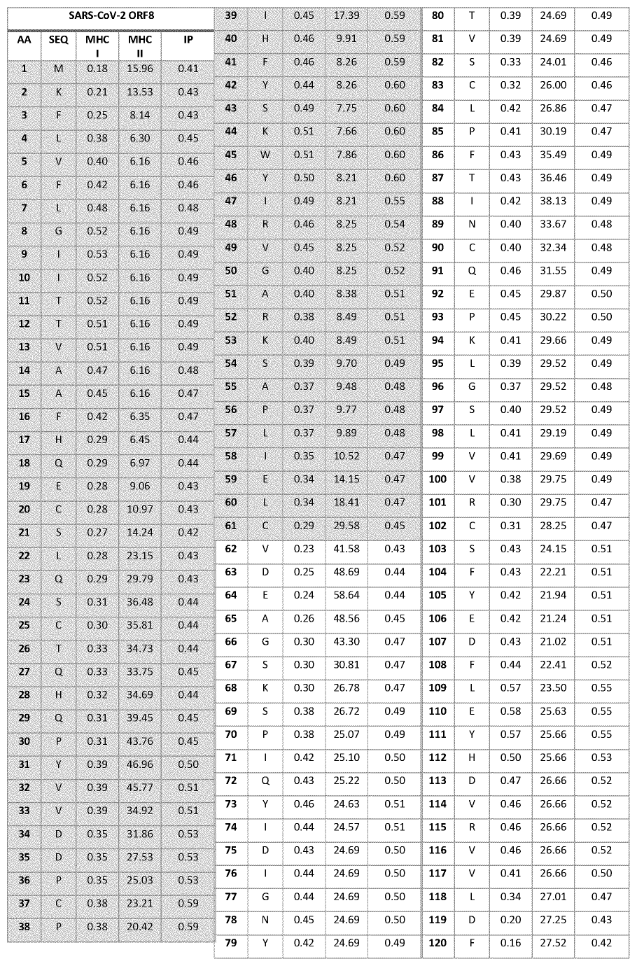

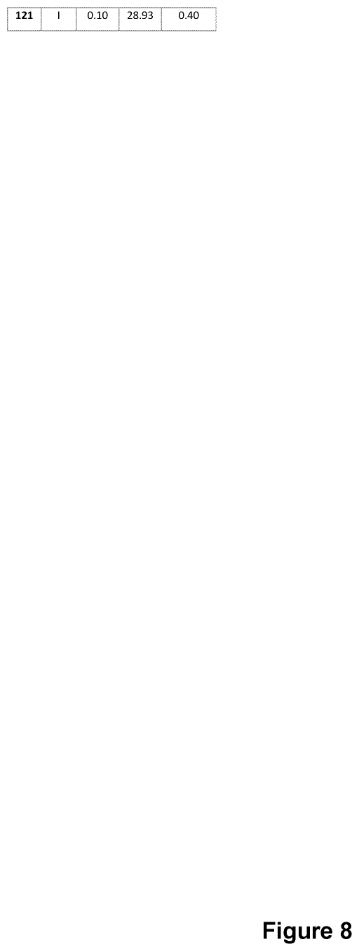

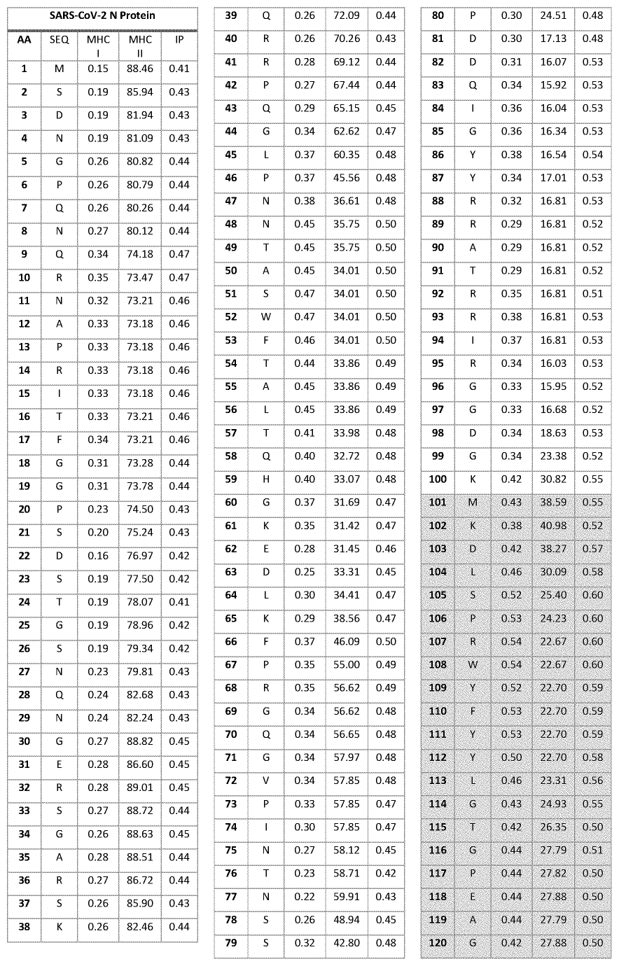

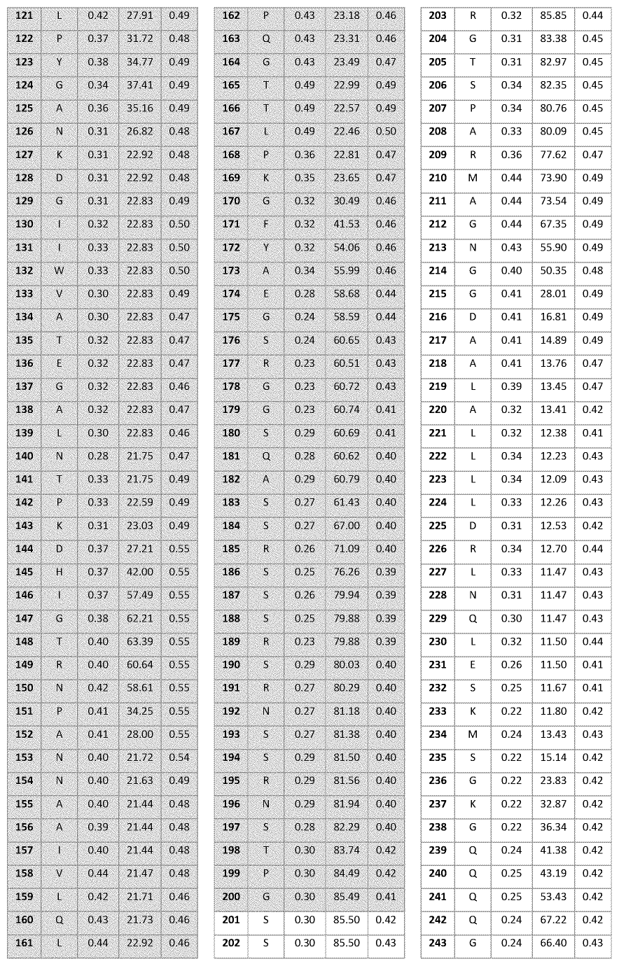

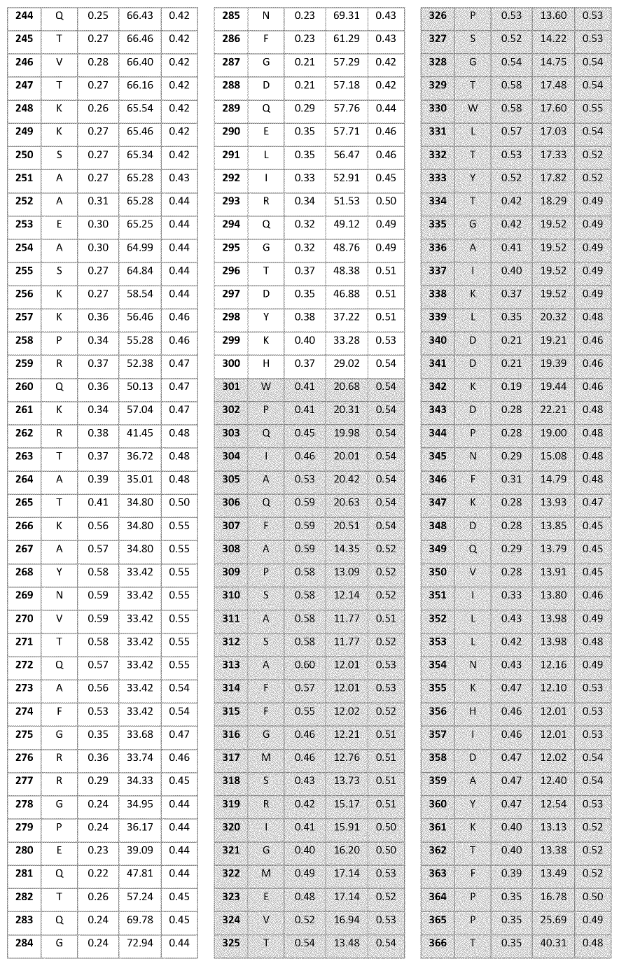

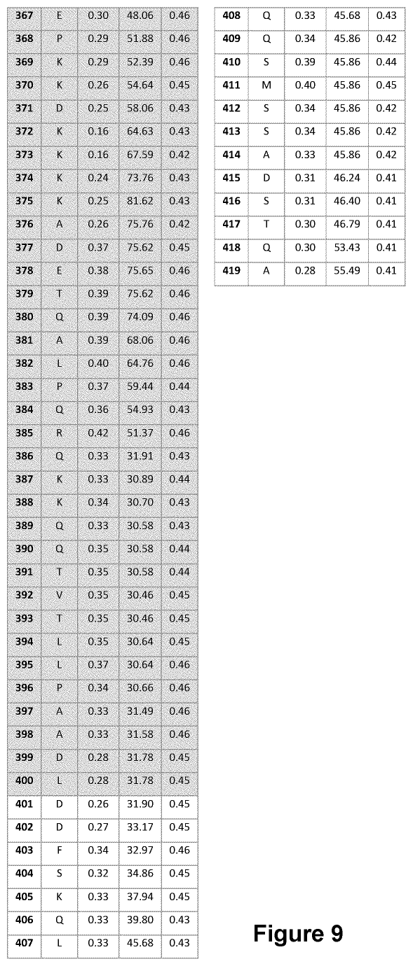

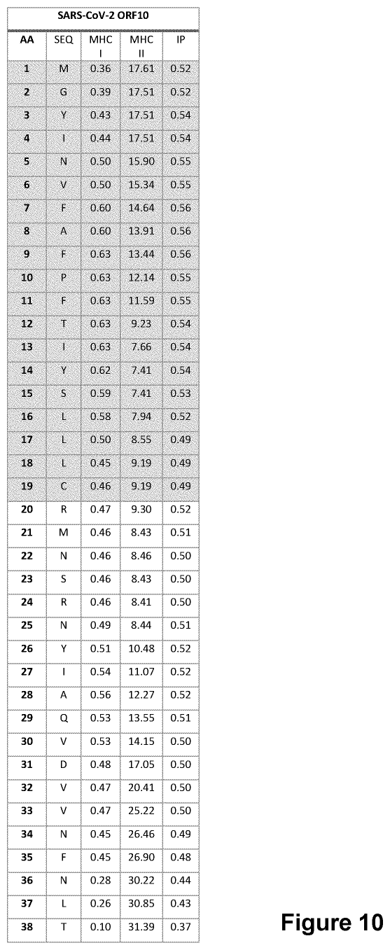

Thus, in a first aspect of the invention, there is provided a coronavirus vaccine composition, comprising one or more epitopes found within any one or more hotspot regions identified in FIGS. 1-10, or a polynucleotide encoding said epitope, wherein each epitope is at least 8 amino acids in length, and wherein each epitope has a mean antigen presentation (AP) cut off value according to the following table:

| | Mean Antigen |
|---|---|
| | Presentation |
| | (AP) |
| | Cut-Off |
| | Value |
| Averaged HLA Type of MHC Class I | ≥0.4 |
| Averaged HLA Type of MHC Class II | ≤13 |

or a mean immune presentation (IP) score of at least 0.5, and wherein an antigen presentation (AP) value is a prediction score assigned to each amino acid as shown in the hotspot regions in FIGS. 1-10, and wherein the mean AP cut-off value is the value, averaged across all amino acids within an epitope, for which said epitope is considered able to stimulate a broad adaptive immune response across a plurality of HLA types, for either MHC Class I and/or MHC Class II immunogenicity.

In the context of the present invention, the term “plurality” is used to refer to “at least two”, or “two or more”.

It is envisaged that the coronavirus vaccine composition of the present invention may be used against any coronavirus infection. Coronaviruses, from the family Coronaviridae, are a group of enveloped, positive-sense single-stranded RNA ((+ssRNA) viruses which can cause respiratory tract infections in human hosts. Mild coronavirus infections include some cases of the common cold, whilst more lethal species of coronavirus such as severe acute respiratory syndrome-related coronavirus (SARS-CoV), Middle East respiratory syndrome-related coronavirus (MERS-CoV), and severe acute respiratory syndrome-related coronavirus 2 (SARS-CoV-2), can cause the more serious diseases SARS, MERS, and COVID-19, respectively. It is proposed that SARS-CoV-2 shares zoonotic origins and close genetic similarity with SARS-CoV, and as such much of our understanding of COVID-19, as well as the research and development of potential prophylactic and therapeutic treatments, has come from the analysis of such other coronaviruses.

SARS-CoV-2 is the causative viral agent behind the 2019-2020 pandemic of COVID-19, a respiratory syndrome characterised by high fever, malaise, rigors, headache, dry cough, lymphopenia and progression to interstitial infiltration in lungs with an eventual mortality of greater than 10% in many countries. SARS-related pathologies of the lungs involve the subsequent stages of viral replication, immune system hyperactivation, and pulmonary destruction (Weis & Navas-Martin 2005, Microbiol Mol Biol Rev. 69 (4): 635-64) and inflammatory exudates in the lungs.

Coronaviruses, such as SARS-CoV-2, attach to their specific cellular receptors via the viral spike protein-invading cells lining the respiratory tract. The receptor for the SARS-CoV-2 virus, a positive single stranded RNA ((+) ssRNA) coronavirus, was identified as angiotensin-converting enzyme 2 (ACE2): a zinc metalloprotease (Li et al. 2003, Nature 426:450-454). Diseased lungs show diffuse alveolar damage, epithelial cell proliferation, and an increased number of macrophages. Further, multinucleate giant-cell infiltrates of macrophage or epithelial cells with syncytium-like cell formation have been described. In addition to hemophagocytosis in the lung, lymphopenia and white-pulp atrophy of the spleen have been observed in SARS patients. At present, most COVID-19 patients receive traditional supportive care such as breathing assistance and/or steroid therapy.

It is envisaged that the vaccine composition of the present invention may aid in the therapeutic or prophylactic treatment of a SARS-CoV-2 infection, or COVID-19, in a human subject, wherein said composition comprises one or more epitopes of the present invention that are capable of stimulating a broad adaptive immune response across a variety of HLA types.

The term “prophylactic treatment”, as used herein, refers to a medical procedure whose purpose is to prevent, rather than treat or cure, a viral infection. In the present invention, this applies particularly to the vaccine composition. The term “prevent” as used herein is not intended to be absolute and may also include the partial prevention of the viral infection and/or one or more symptoms of said viral infection. In contrast, the term “therapeutic treatment” refers to a medical procedure with the purpose of treating or curing a viral infection or the associated symptoms thereof, as would be appreciated within the art.

The term vaccine composition, or vaccine, which from herein may be referred to interchangeably as the “composition”, relates to a biological preparation that provides active acquired immunity to a particular infectious disease, in this case a coronavirus infection. Typically the vaccine contains an agent, or “foreign” agent, that resembles the infection-causing virus, which within the prior art has often been a weakened or killed form of said virus, or one or more of its surface proteins such as the spike(S) protein or other associated proteins (Williamson et al. 1995, FEMS Immunology and Medical Microbiology 12 (3-4): 223-230). Such a foreign agent would be recognised by a vaccine-receiver's immune system, which in turn would destroy said agent and develop “memory” against the virus, inducing a level of lasting protection against future viral infections from the same or similar sub-species. Through the route of vaccination, including those vaccine compositions of the present invention, it is envisaged that once the vaccinated subject again encounters the same virus or viral isolate of which said subject was vaccinated against, the individual's immune system may thereby recognise said virus or viral isolate and elicit a more effective defence against infection. A more in-depth description of types of vaccines within the art can be found in U.S. Pat. No. 6,541,003 B1, which is hereby incorporated as reference.

The active acquired immunity that is induced may be humoral and/or cellular. Humoral immunity refers to a response involving B cells which produce antibodies that specifically bind to antigens, or any future antigens, corresponding to those within the administered vaccine composition. B cells, each expressing a unique B cell receptor (BCR), recognise antigens in their native form, such as the tertiary structure of a SARS-CoV-2 spike protein. Upon this recognition and further interaction with other cells of the immune system, the activated B cell can differentiate into a plasma cell specialised to secrete antibodies against the encountered antigen. The term antibody refers to an immunoglobulin (Ig) that is used by the immune system to specifically identify and neutralize foreign antigens. A subset of these B-cell derived plasma cells become long-lived antigen-specific memory B cells, as would be well understood by the skilled person.

Cellular immunity, meanwhile, can be broken into two distinct arms. The first involves helper T cells, or CD4+ T cells, which produce cytokines and orchestrate the activity of other immune cells in the immune response. The second involves killer T cells, also known as cytotoxic T lymphocytes (CTLs), or CD8+ T cells, which are cells capable of recognising antigens/epitopes presented by HLA and eradicate viral or bacterial infected host cells. In contrast to B cells, T cells only recognise antigens that have been processed into peptides and have been loaded onto histocompatibility complex (MHC) molecule and presented at the cell surface. CD4+ T cells interact with MHC class II molecules (MHC Class II), and are responsible for orchestrating the immune response, recognizing foreign antigens, activating various parts of the immune system and activating B cells and CD8+ T cells. CD8+ T cells interact with MHC Class I receptors and play a role in mounting an immune response against intracellular pathogens. As would be understood by the skilled person, on resolution of the infection, a subset of both CD8+ T cells and CD4+ T cells may remain as memory T cells, contributing to the acquired adaptive immunity, and allowing for a faster and stronger response to any secondary infection from the same foreign body (Bonilla & Oettgen 2010, Journal of Allergy and Clinical Immunology 125:33-40).

It is envisaged that the vaccine composition of the present invention may be an epitope-based vaccine, or in other words, is comprised of one or more epitopes. Epitope-based vaccines (EVs) make use of short antigen-derived peptides corresponding to immune epitopes, which are administered to trigger a protective humoral and/or cellular immune response. EVs potentially allow for precise control over the immune response activation by focusing on the most relevant-immunogenic and conserved-antigen regions. Experimental screening of large sets of peptides is time-consuming and costly; therefore, in silico methods that facilitate T-cell epitope mapping of protein antigens are paramount for EV development. The prediction of T-cell epitopes focuses on the presentation of peptides at the infected cell surface by proteins encoded by the major histocompatibility complex (MHC).

The epitopes of the present invention may interact with MHC Class I and/or MHC Class II molecules to induce a CD8+ T cell and/or CD4+ T cell response, respectively. In a preferred embodiment of the present invention, there may be at least one epitope that interacts with MHC Class I, and at least one epitope that interacts with MHC Class II.

The term “epitope” as used herein refers to any part of an antigen that is recognised by any antibodies, B cells, or T cells. An “antigen” refers to a molecule capable of being bound by an antibody, B cell or T cell, and may be comprised of one or more epitopes. As such, the terms epitope and antigen may be used interchangeably herein. Epitopes may also be referred to by the molecule for which they bind, such as “T cell epitopes”, or more specifically, “MHC Class I epitopes” or “MHC Class II epitopes”. T cell epitopes presented by MHC Class I molecules are typically peptides between 8 and 11 amino acids in length, whereas MHC Class II molecules present longer peptides, and as such epitopes presented by MHC Class II are often 13-17 amino acids in length (Alberts 2002, Molecular Biology of the Cell P. 1401).

The one or more epitopes of the present invention are at least 8 amino acids in length. In some embodiments of the present invention, the one or more epitopes are between 8 and 11 amino acids in length. In other embodiments of the invention, the one or more epitopes are between 8 and 17 amino acids in length and may be 8 to 24 amino acids in length. In further embodiments of the invention, the one or more epitopes may be between 8 and 30 amino acids in length.

It is envisaged that the epitopes may differ in length from each other, and may overlap with each other. For example, the vaccine composition of the present invention may comprise one minimal epitope of 8 amino acids in length, in addition to a further epitope of 25 amino acids in length, wherein said epitope of amino acids in length may overlap with part of, or fully comprise the entirety of, the first epitope of 8 amino acids in length.

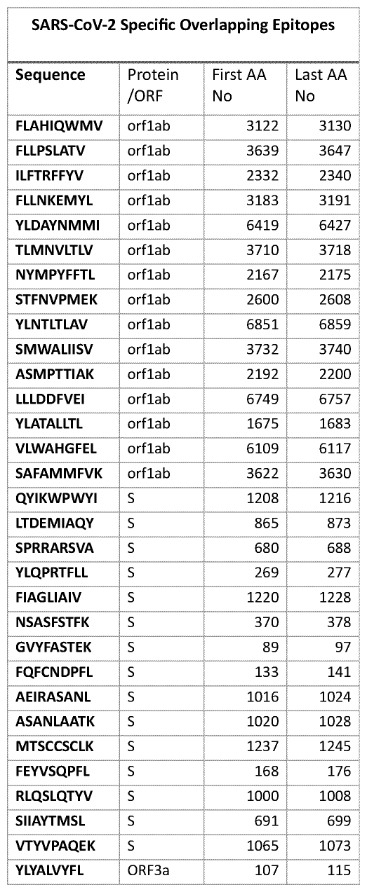

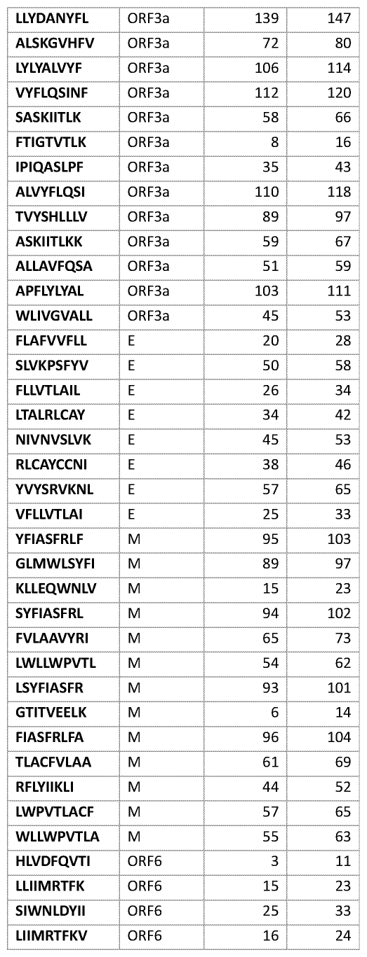

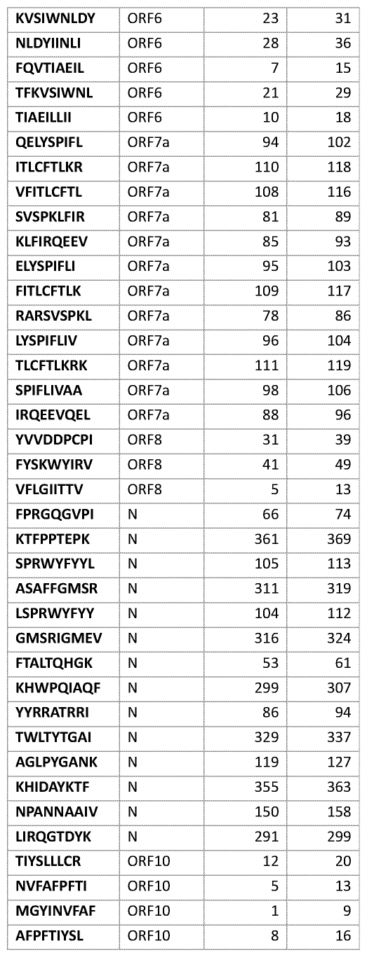

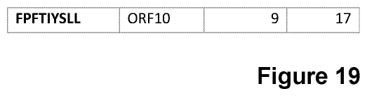

Thus in some embodiments of the present invention, the one or more epitopes may have the same length, or same number of amino acids. In other embodiments, the one or more epitopes may differ in length, or the number of amino acids. In some embodiments, the one or more epitopes may overlap with each other at least partly. In other embodiments, the one or more epitopes may overlap across more than one hotspot. A list of particularly preferred epitopes that may overlap with more than one hotspot can be found in FIG. 19.

In other embodiments, one of the epitopes may fully comprise the entirety of another epitope within the same composition. Various “hotspot” regions containing one or more epitopes are identified herein and, as explained in more detail below, can be utilised in the vaccine composition to present the epitopes. Accordingly, the invention encompasses a vaccine composition made up from one or more hotspot regions, each hotspot containing one or more epitopes as defined herein.

It is envisaged that the one or more epitopes of the present invention are capable of stimulating a broad adaptive immune response across a plurality of human leukocyte antigen (HLA) types. The human leukocyte antigen (HLA) system is a complex of genes encoding the MHC proteins in humans. Owing to the highly polymorphic nature of HLA genes, in which the term “polymorphic” refers to a high variability of different alleles, the precise MHC proteins of each human individual coded by varying HLA genes may differ to fine-tune the adaptive immune system. Many thousands of different alleles have been recognised for HLA molecules. As a result, each individual may have a unique “HLA type”, or HLA phenotype, that differs across the global population, with a slight variability in the functioning of the immune system. The terms HLA type, HLA allele, or HLA phenotype may be used interchangeably herein. HLA types are of particular significance when considering a vaccine comprised of epitopes that interact with MHC class I or class II molecules, as many epitopes are restricted in their capability of binding only particular HLA molecules encoded by particular HLA alleles, or in other words, restricted to certain HLA types only. It would thus be appreciated by the skilled person that T cell epitopes that are capable of binding to a subject's MHC Class I or MHC Class II molecules (and be presented at the infect cell surface), compatible with said subject's HLA type, would thus present as a robust vaccine. A vaccine composition consisting of the same T cell epitopes may not prove effective if given to a subject with a different HLA type, if said HLA type encodes MHC molecules that are not capable of interacting with said T cell epitopes. Such epitopes would not be able to stimulate a broad adaptive immune response across for either MHC Class I and/or MHC Class II immunogenicity in that particular subject.

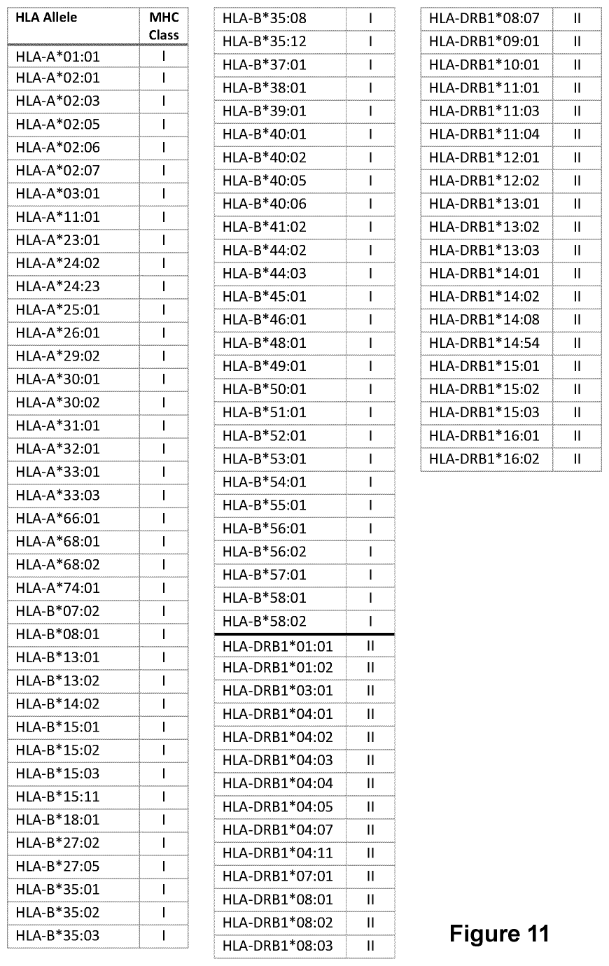

The epitopes of the present invention, in contrast, have been identified to be able to stimulate a broad adaptive immune response across a plurality of HLA types, including alleles such as HLA-A*24:02 and HLA-DRB1*01:01. The HLA alleles as referenced herein are given contemporary HLA nomenclature as standard to the field, wherein HLA-A, for example, refers to the gene loci in chromosome 6, whilst HLA-A*24:02 refers to the protein the allele codes for. An in depth explanation of the complexities of HLA nomenclature can be found in Marsh et al. 2010, Tissue Antigens 75 (4): 291-455. The artificial intelligence (AI)-driven approach of the present invention analysed all 100 of the most frequent HLA-A and HLA-B Class I and HLA-DR Class II alleles in the human population, as shown in FIG. 11.

The AI-driven platform used to identify and predict the one or more epitopes of the present invention was surprisingly robust, as was its integrated statistical analysis. Firstly, epitope mapping of the SARS-CoV-2 virus proteome for Class I epitopes was carried out using cell-surface antigen presentation and immunogenicity predictors from the “NEC Immune Profiler” suite of tools. Antigen Presentation (AP) was predicted from a machine learning model that integrates in an ensemble machine learning layer information from several HLA binding predictors-trained using empirically measured binding affinity data- and 13 different predictors of antigen processing.

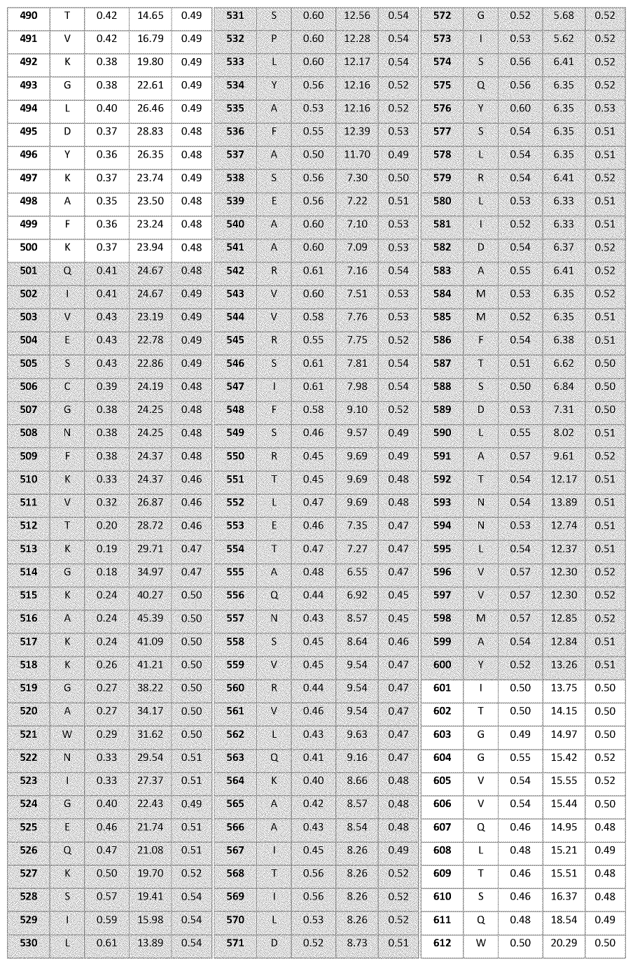

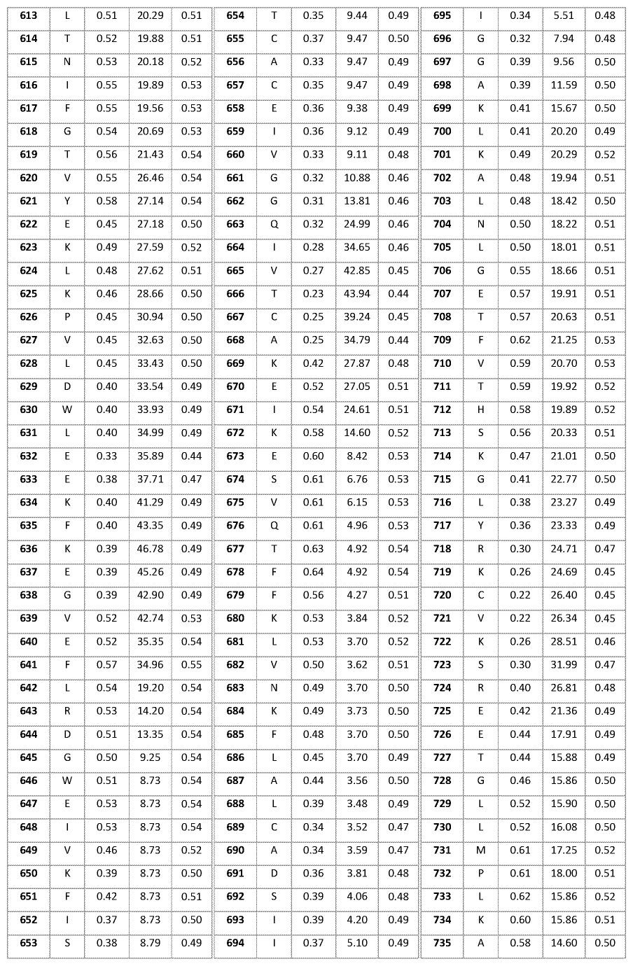

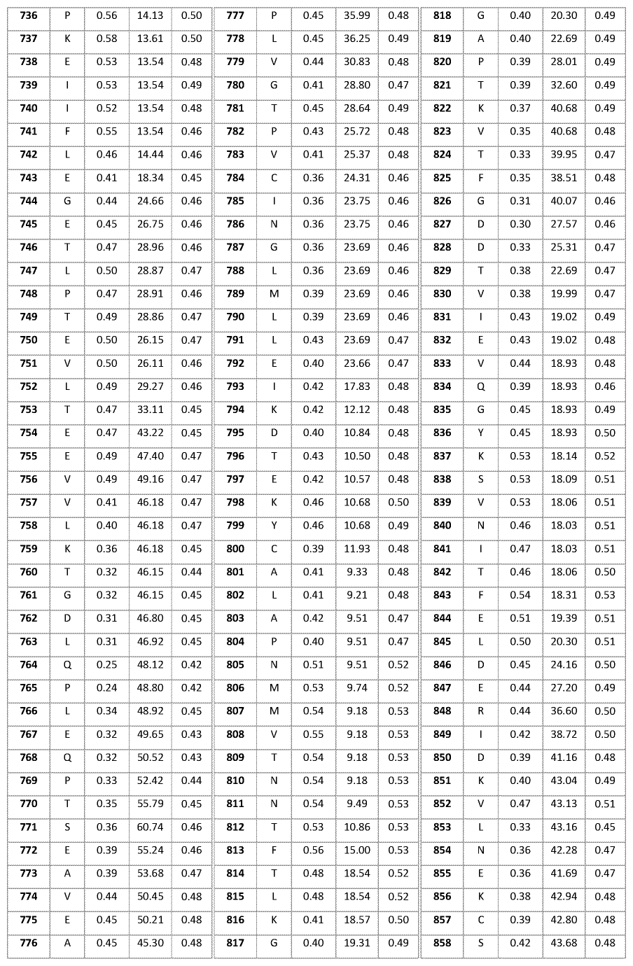

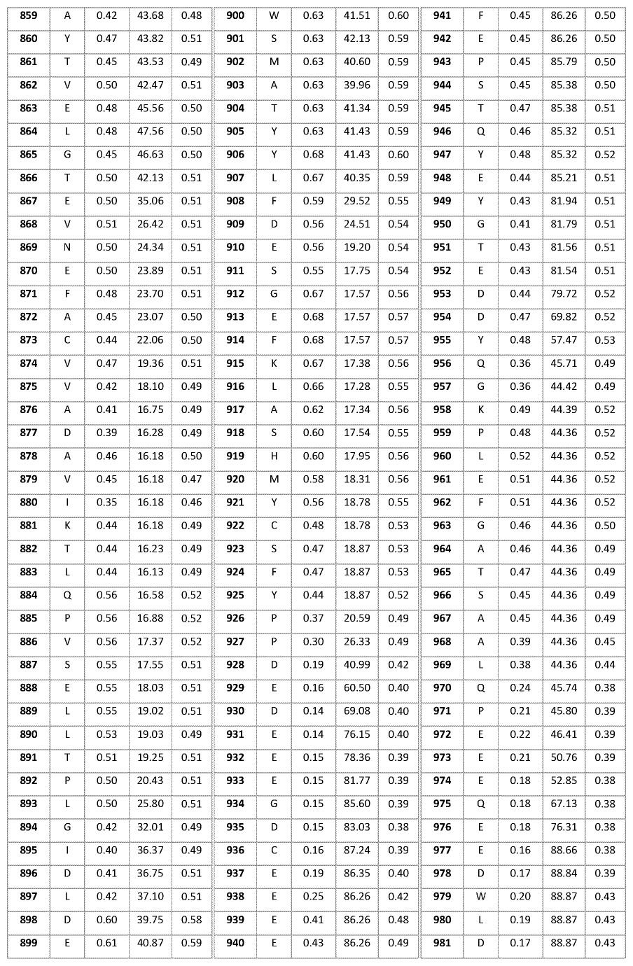

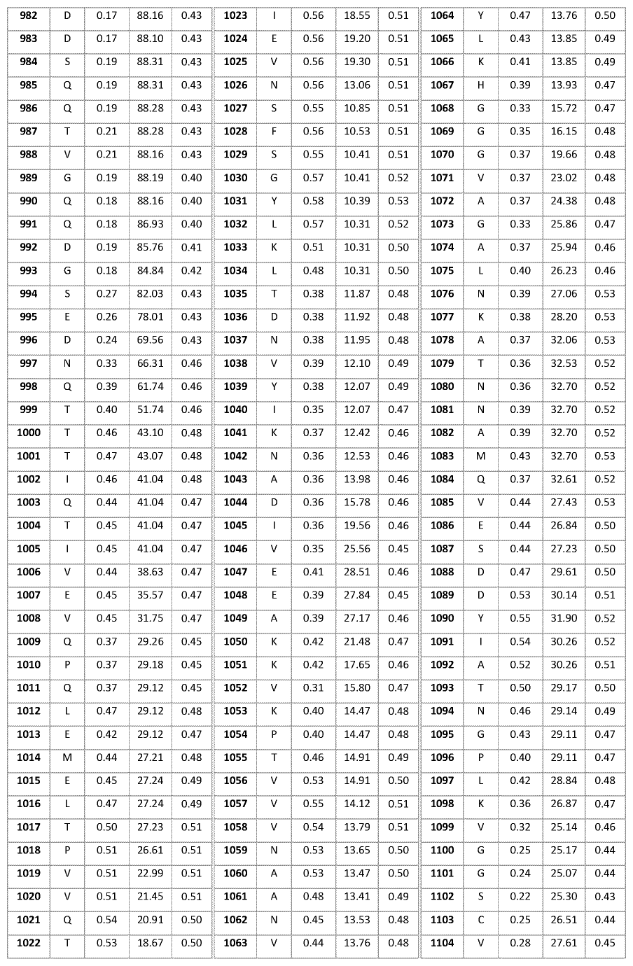

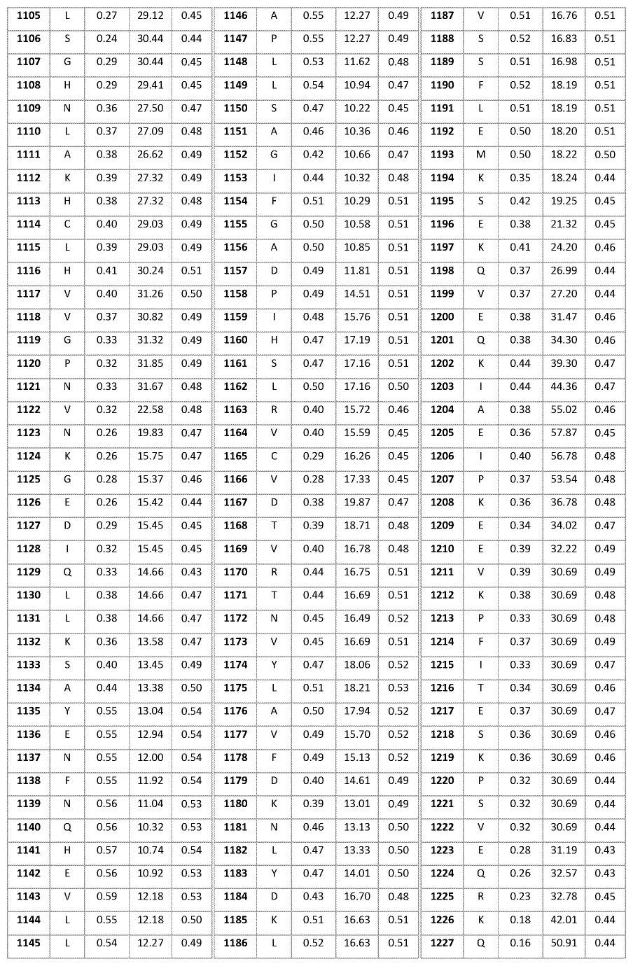

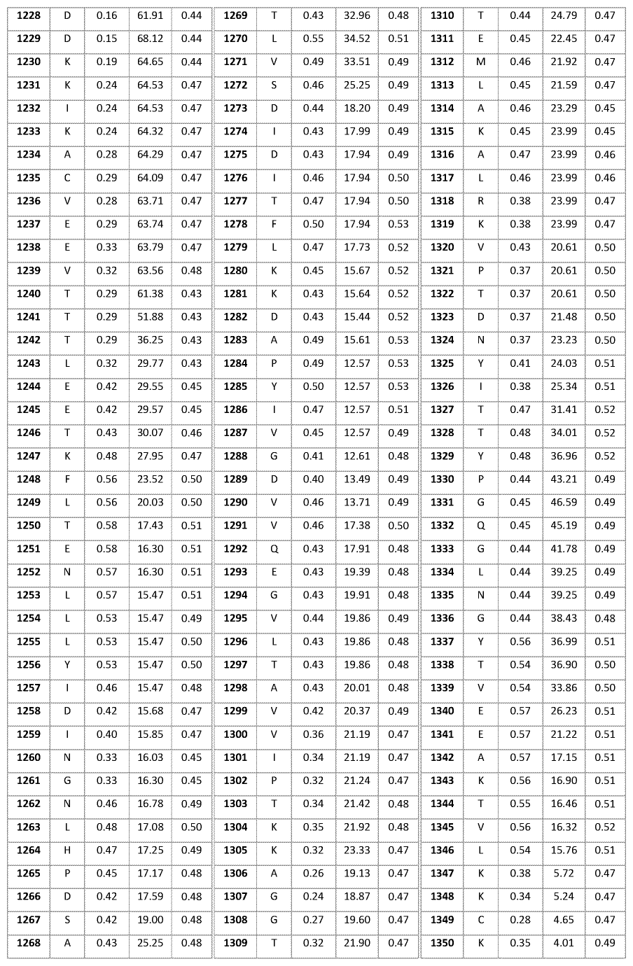

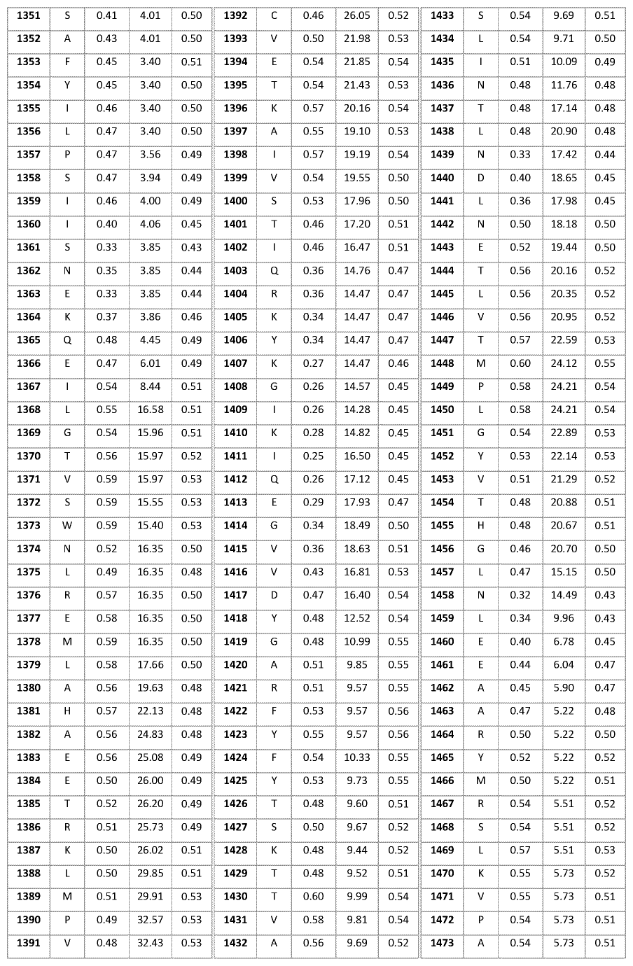

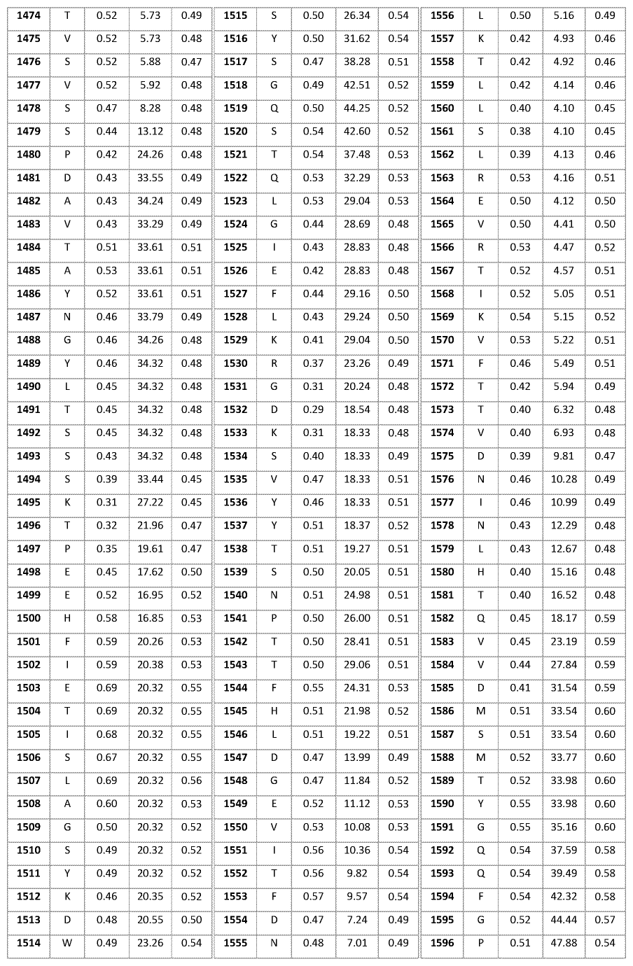

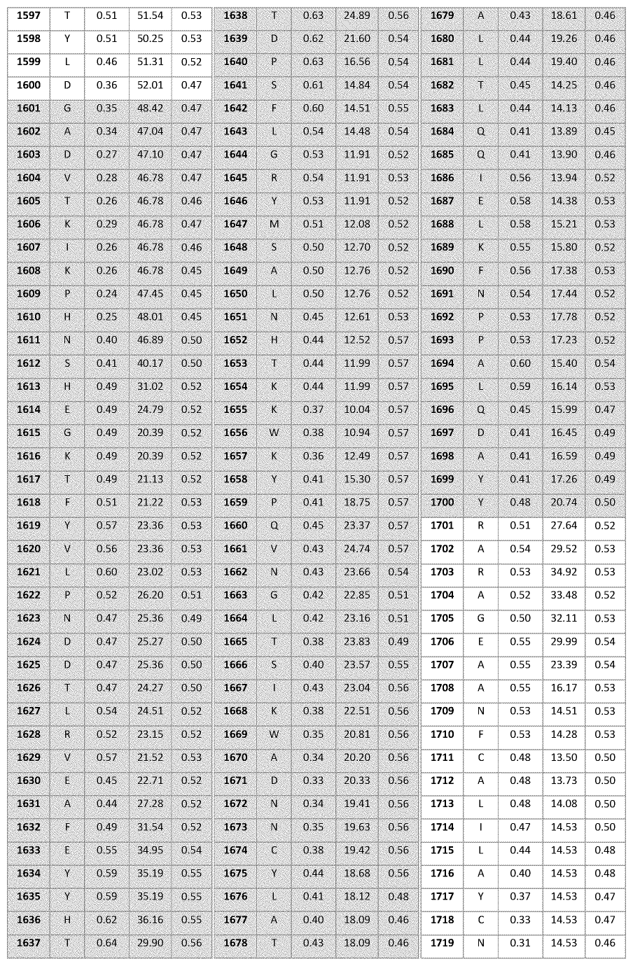

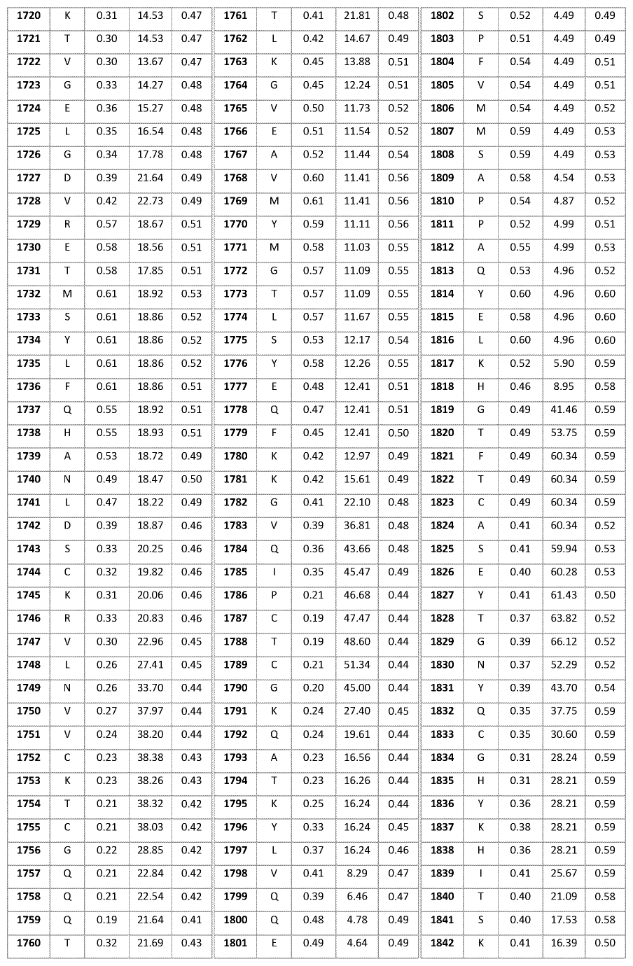

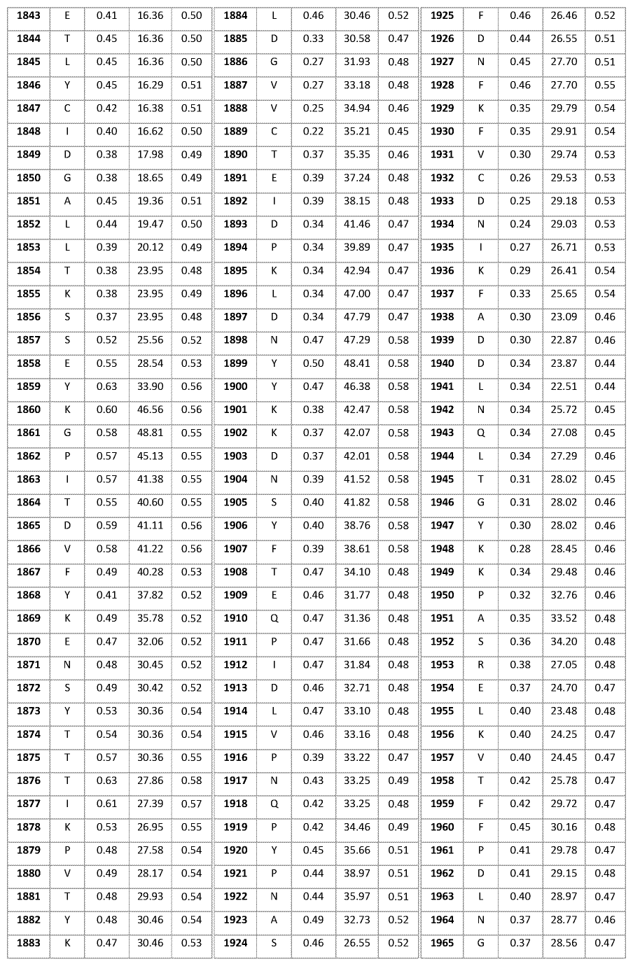

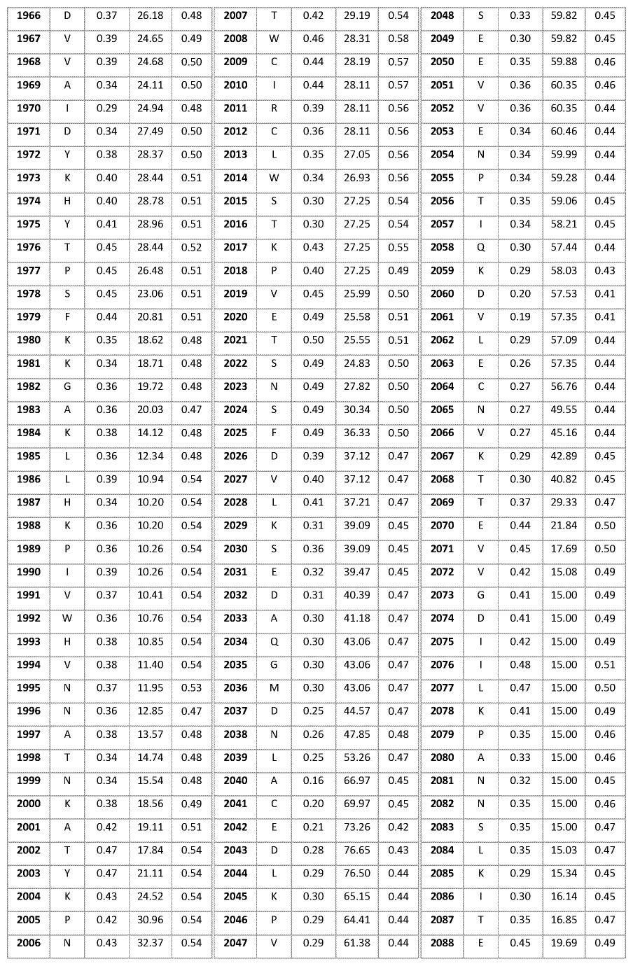

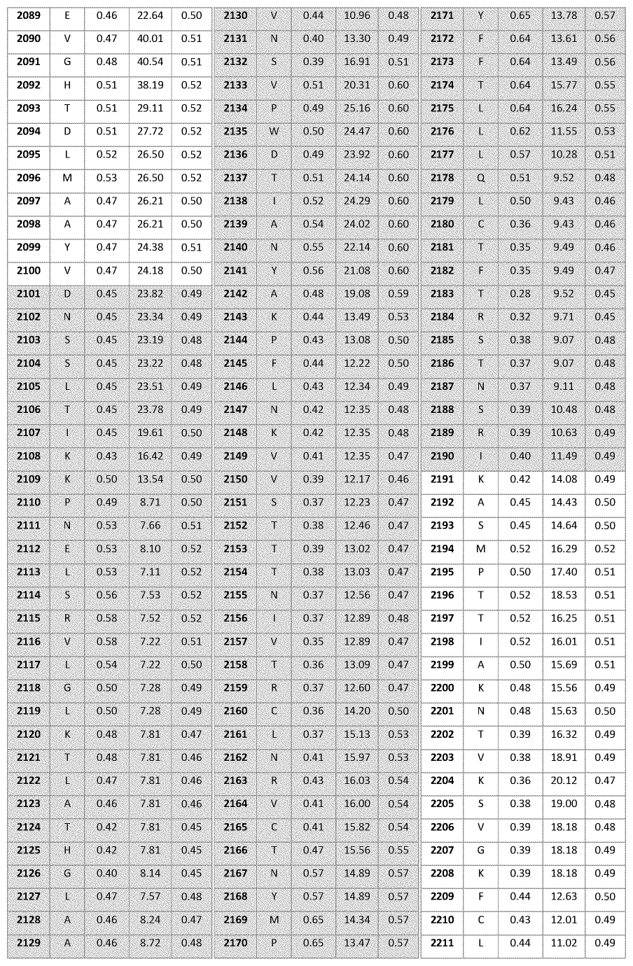

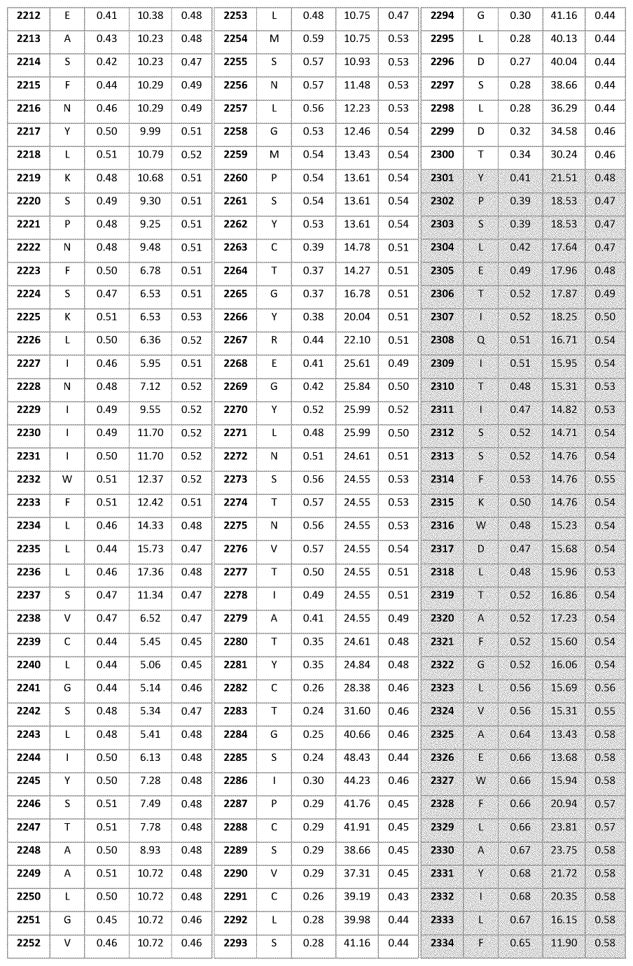

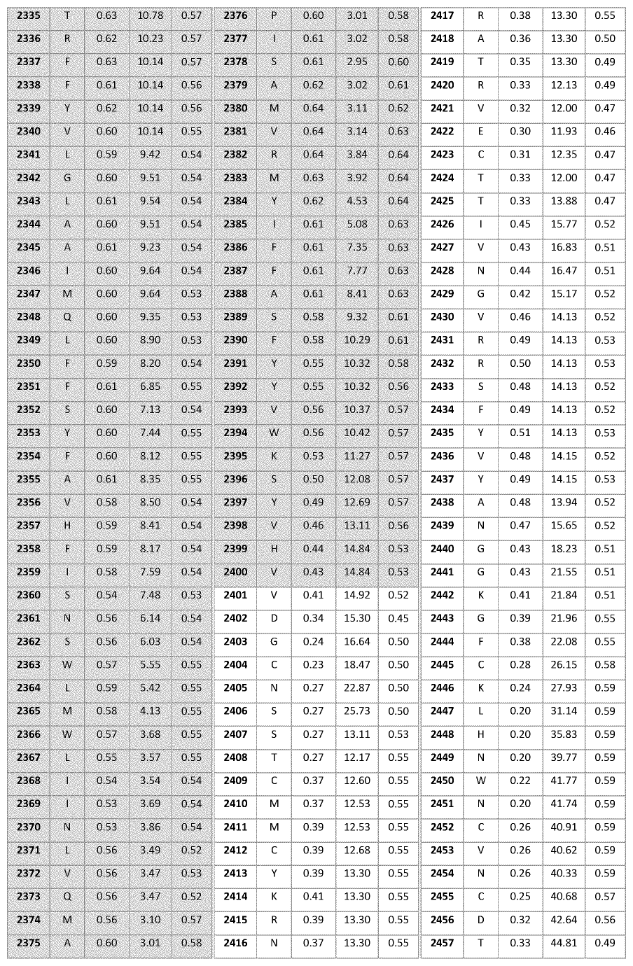

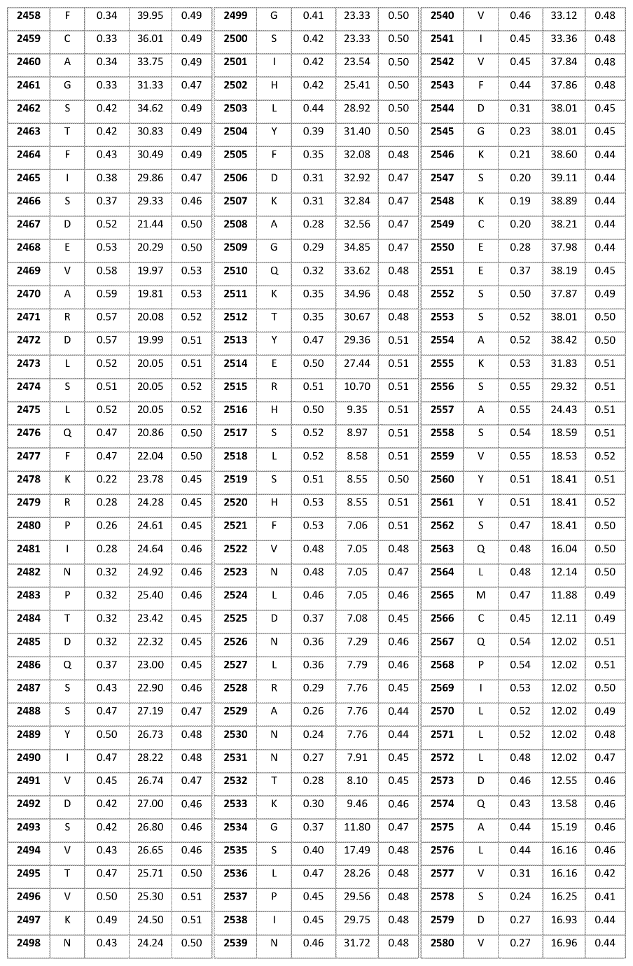

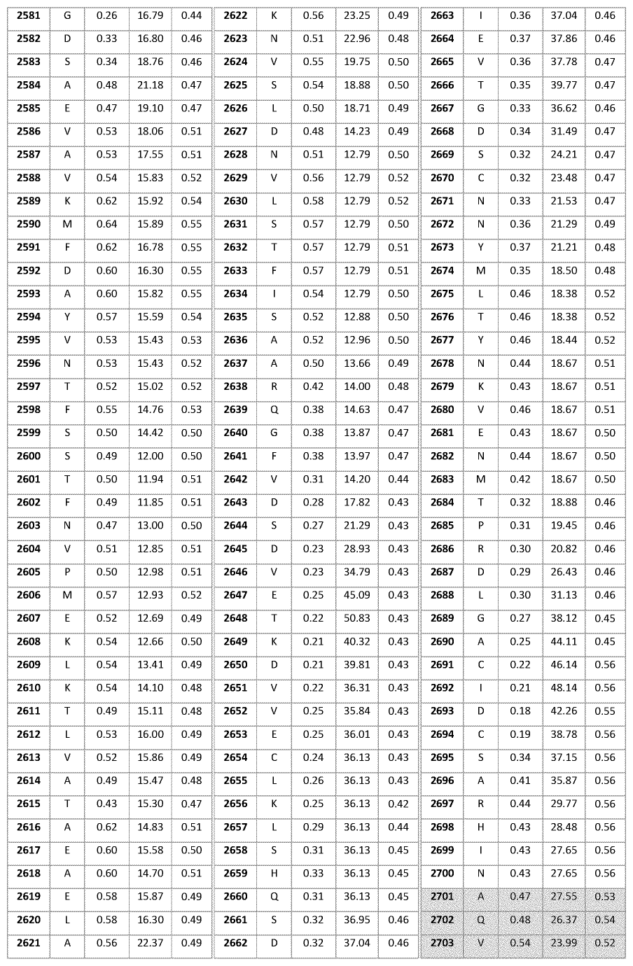

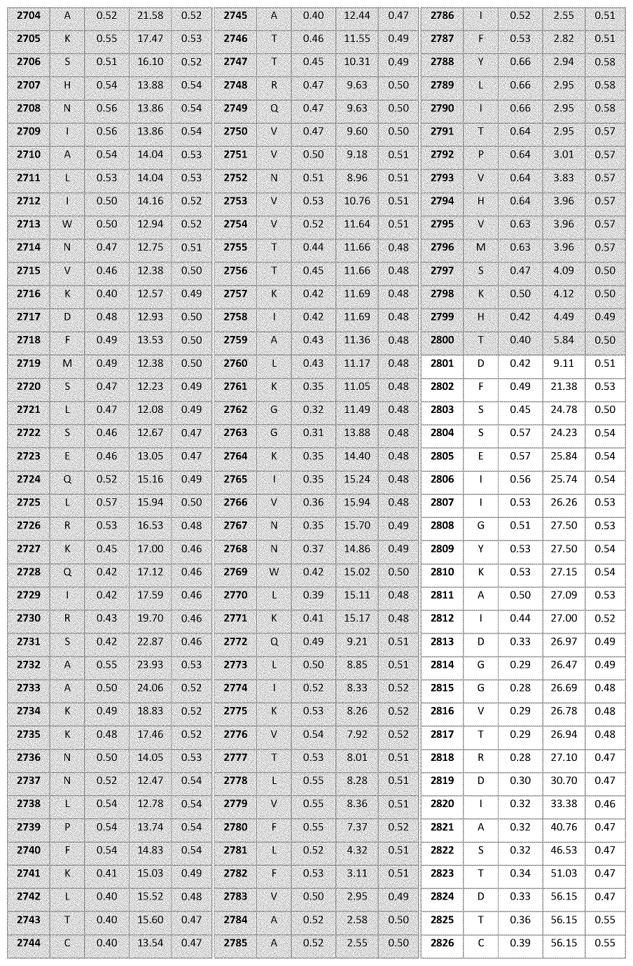

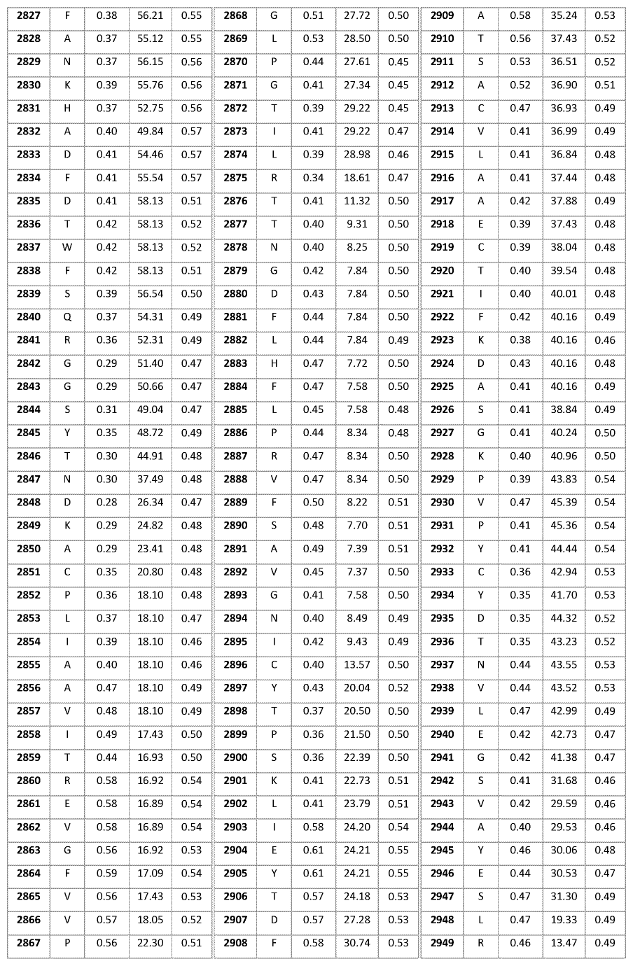

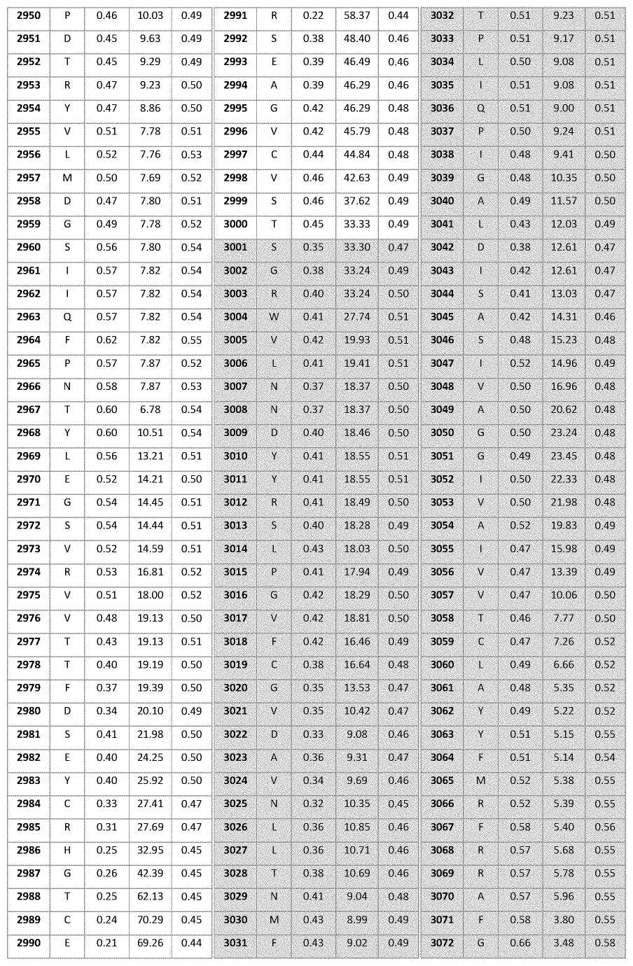

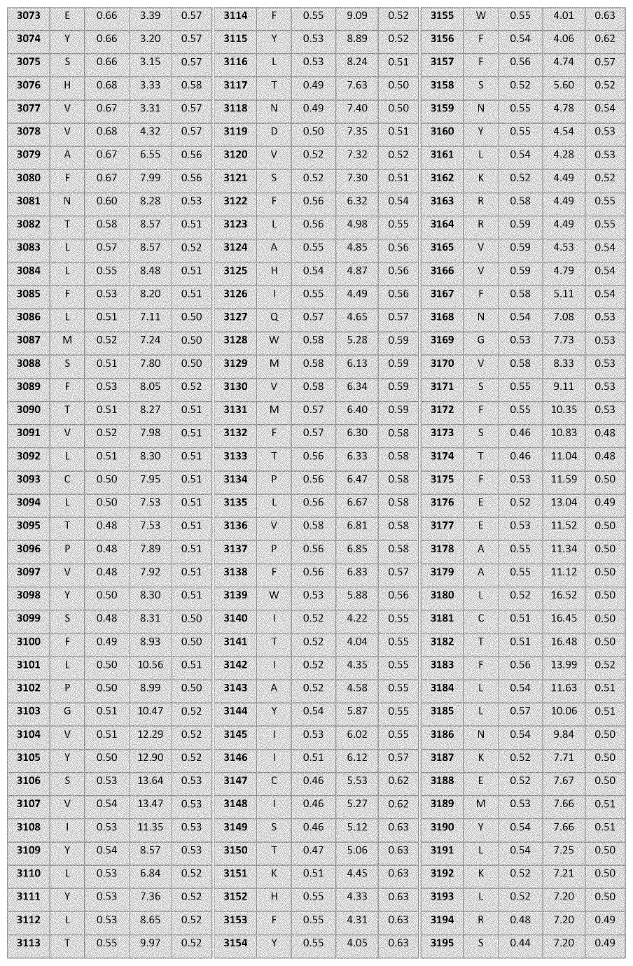

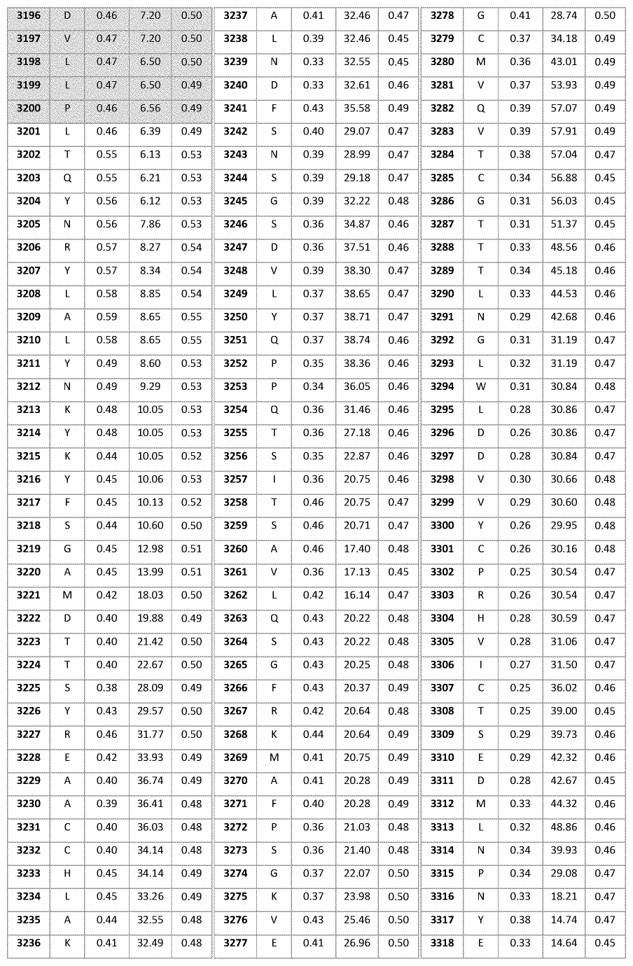

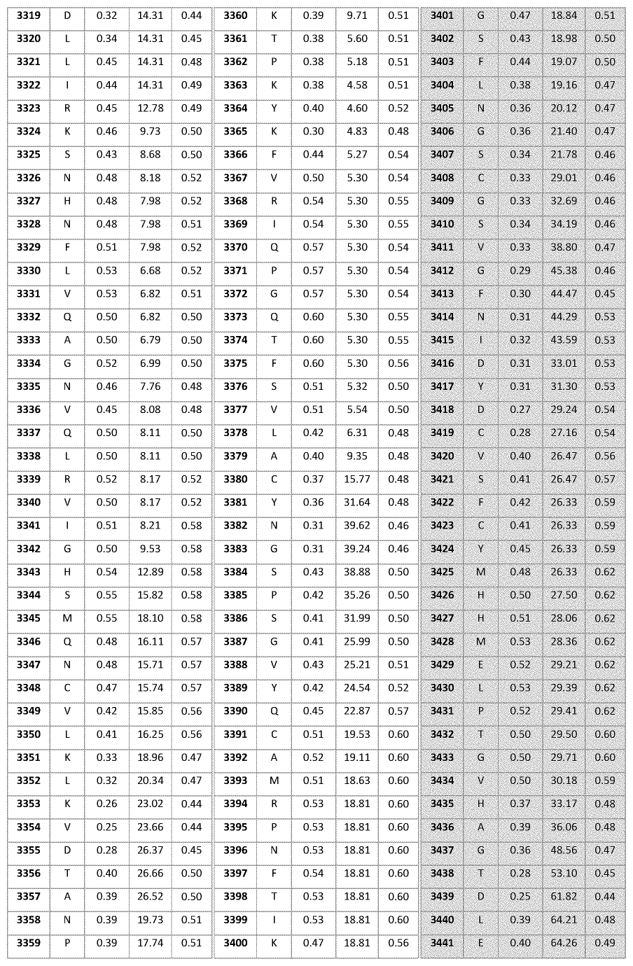

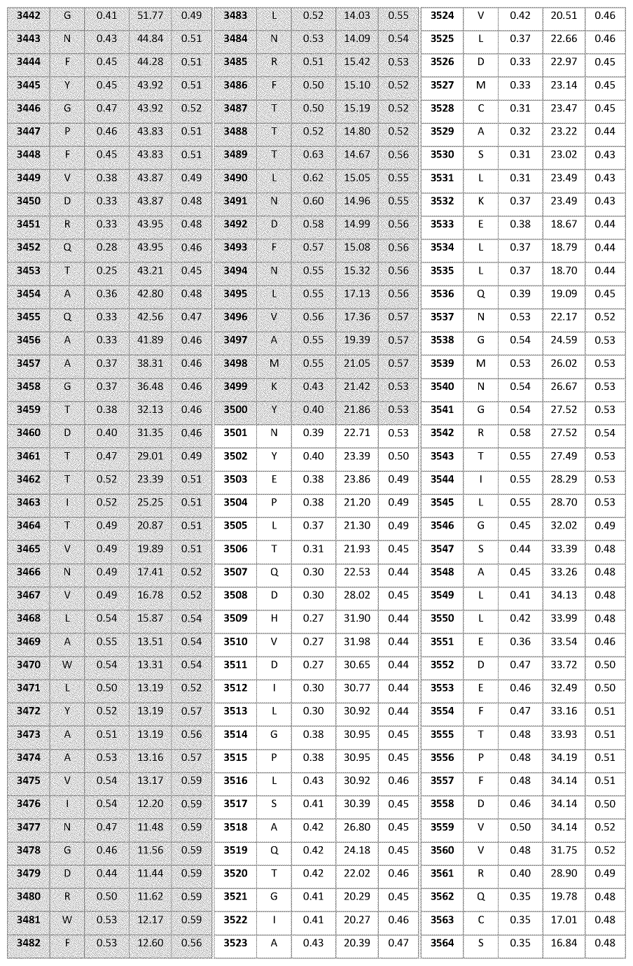

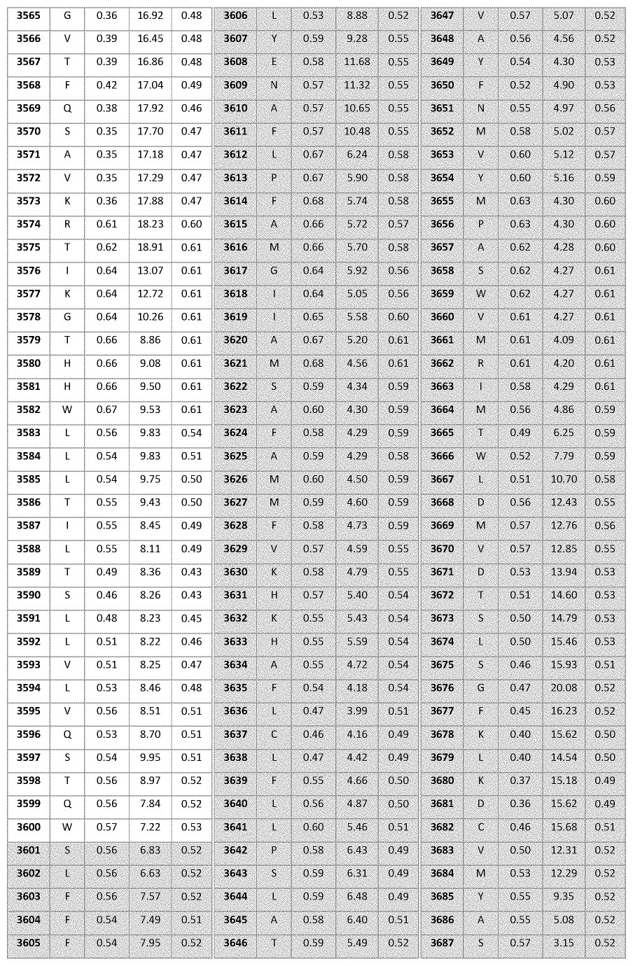

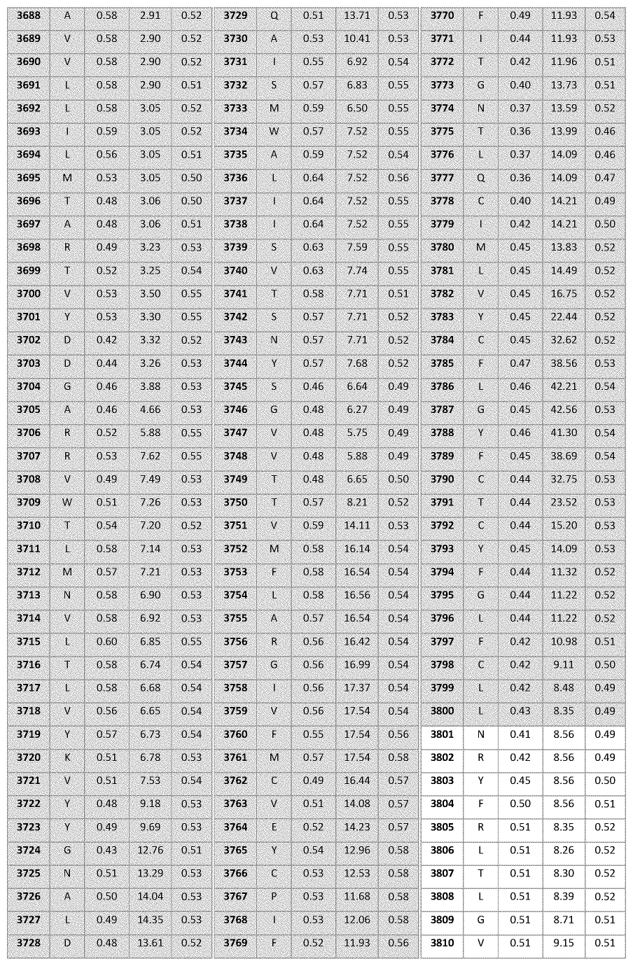

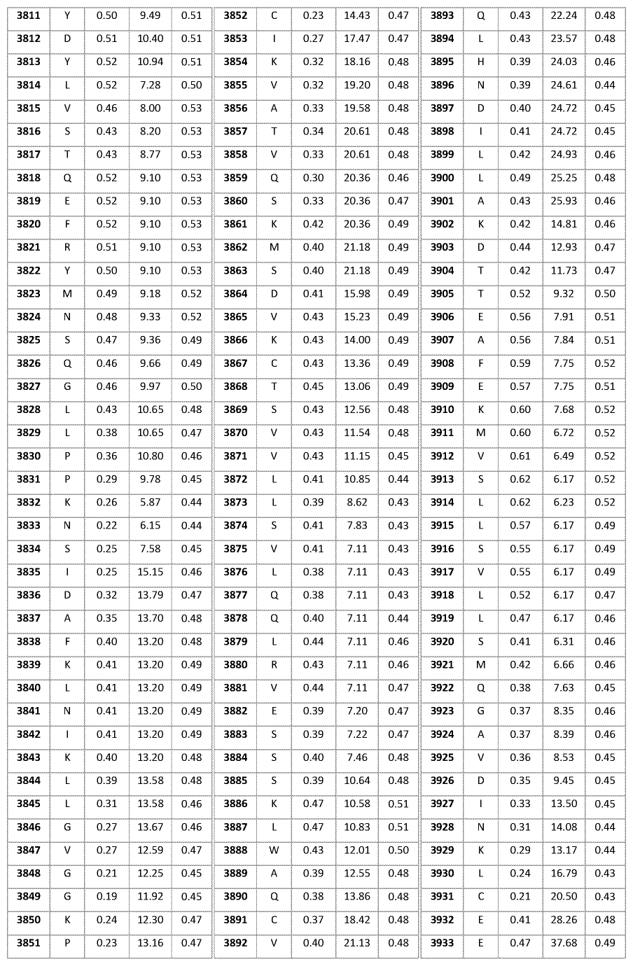

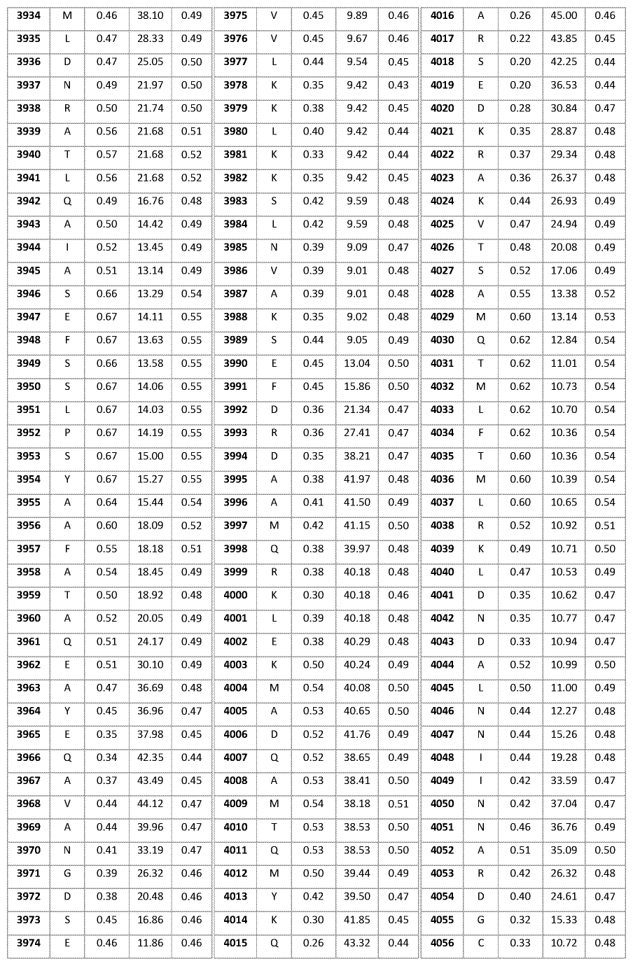

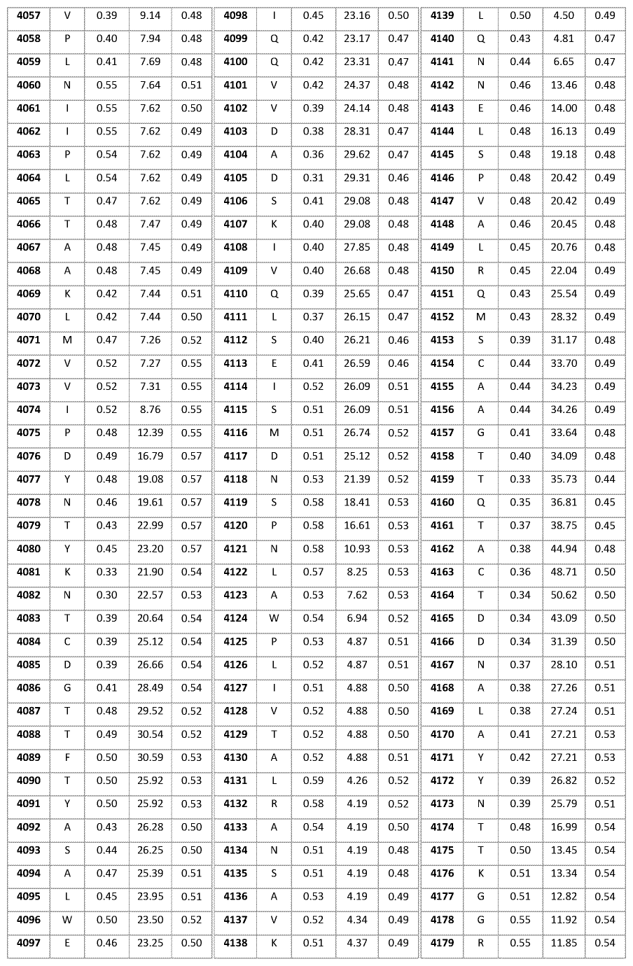

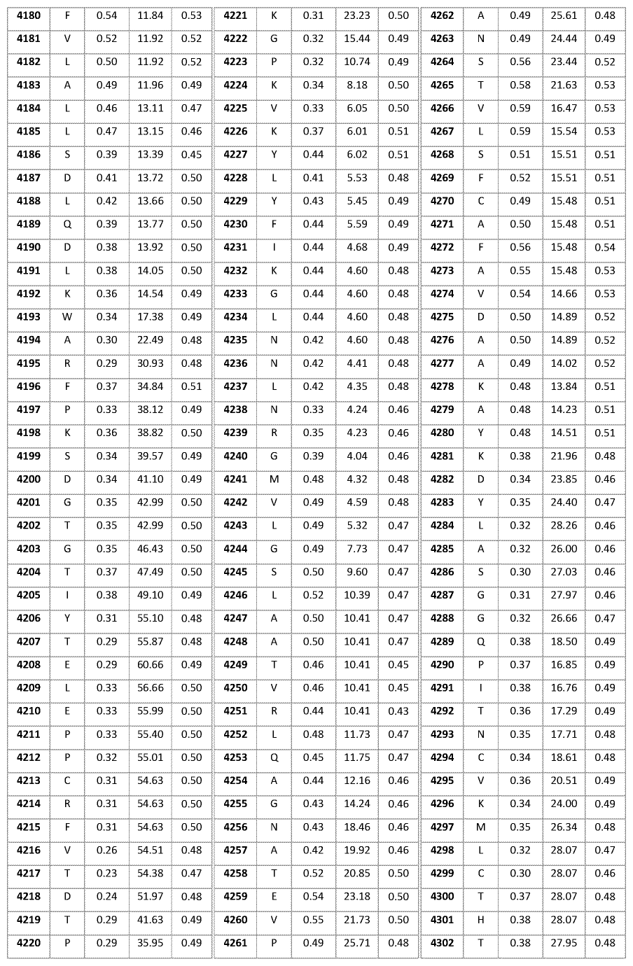

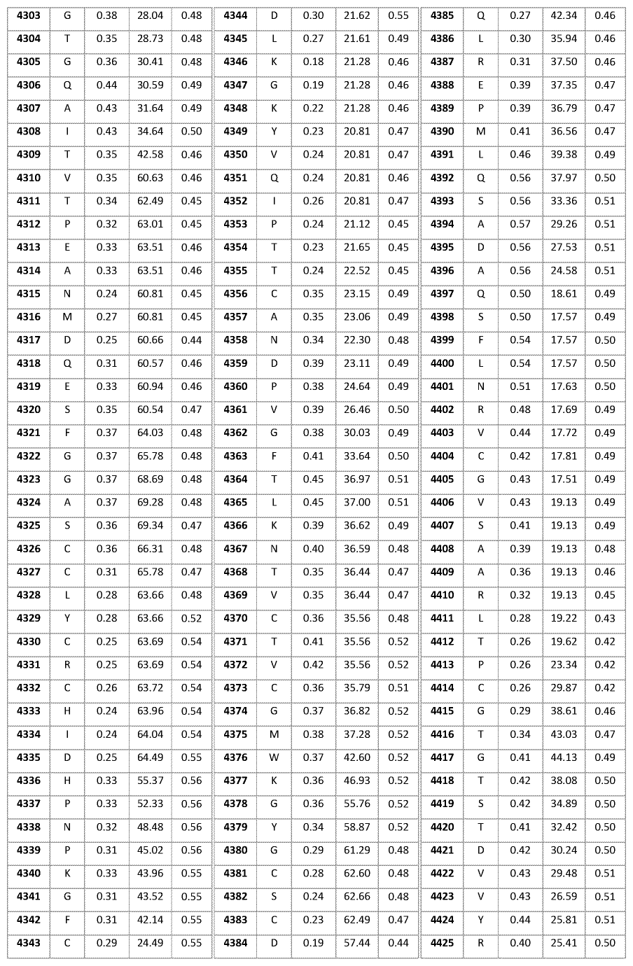

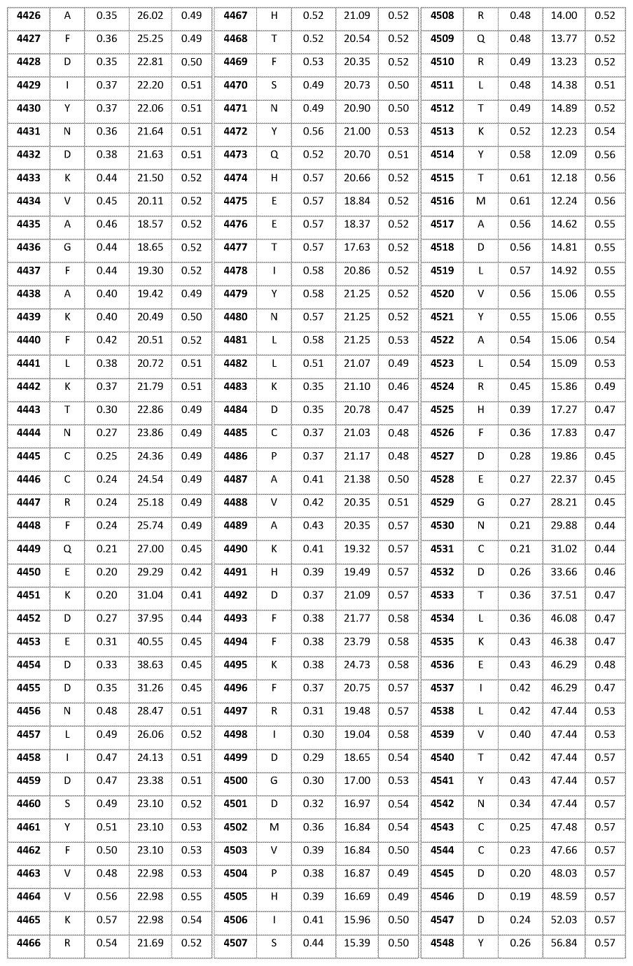

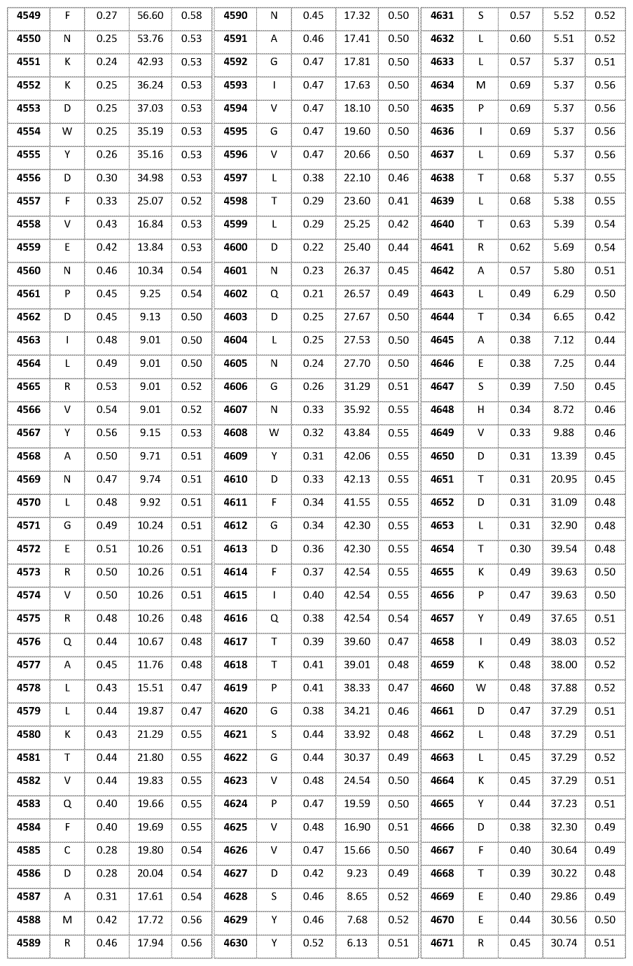

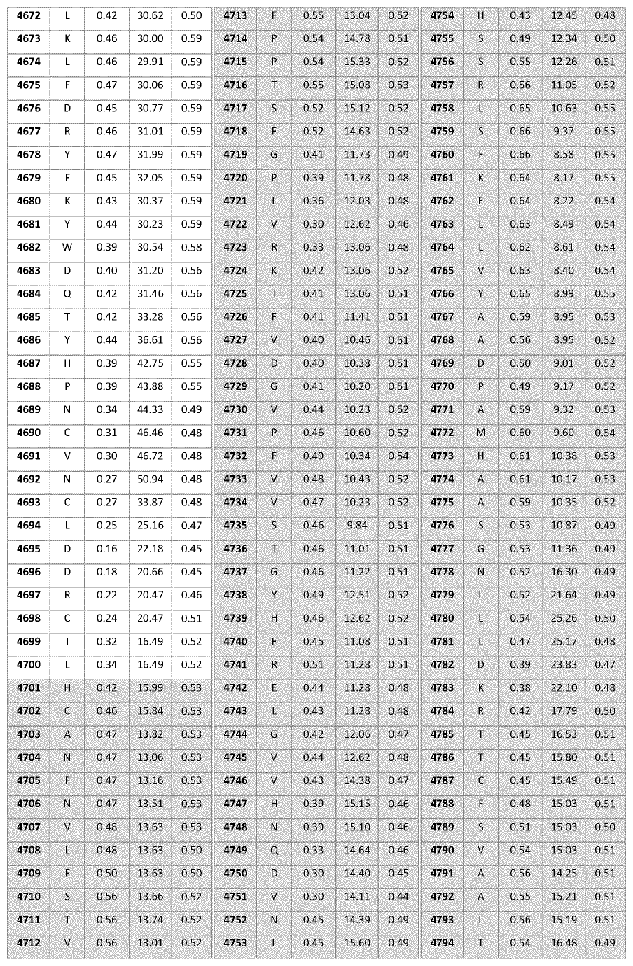

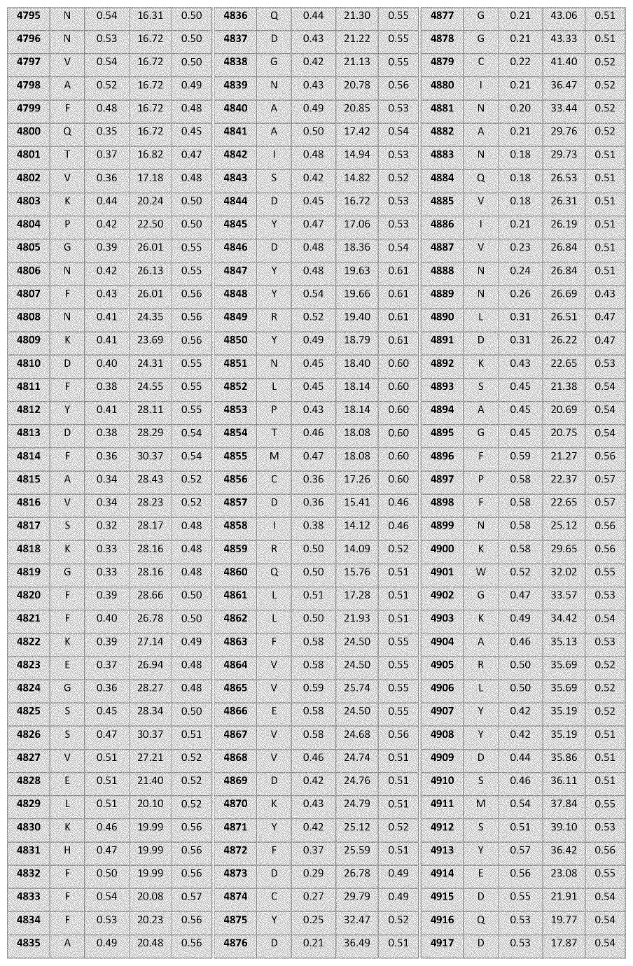

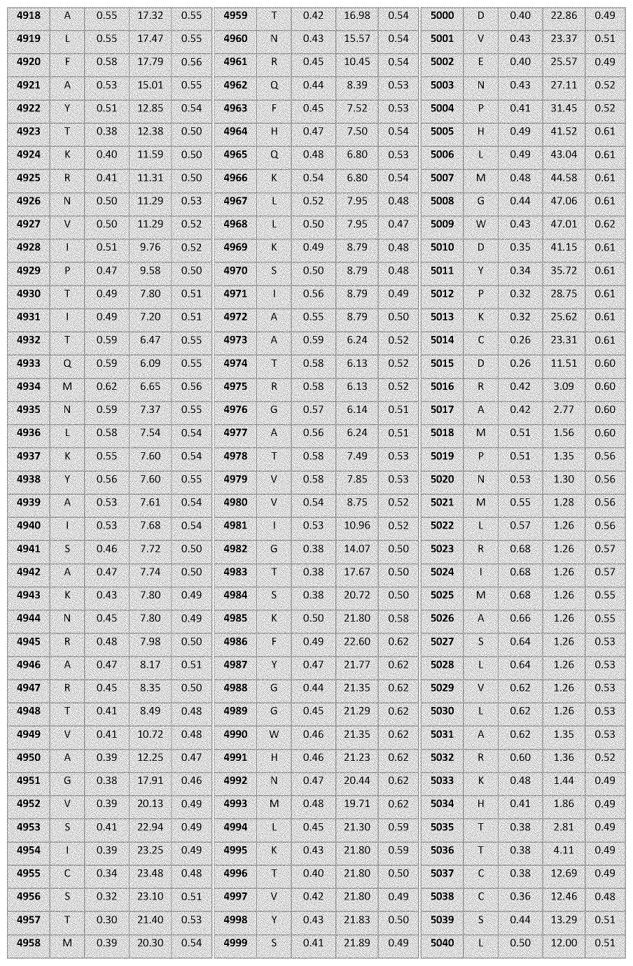

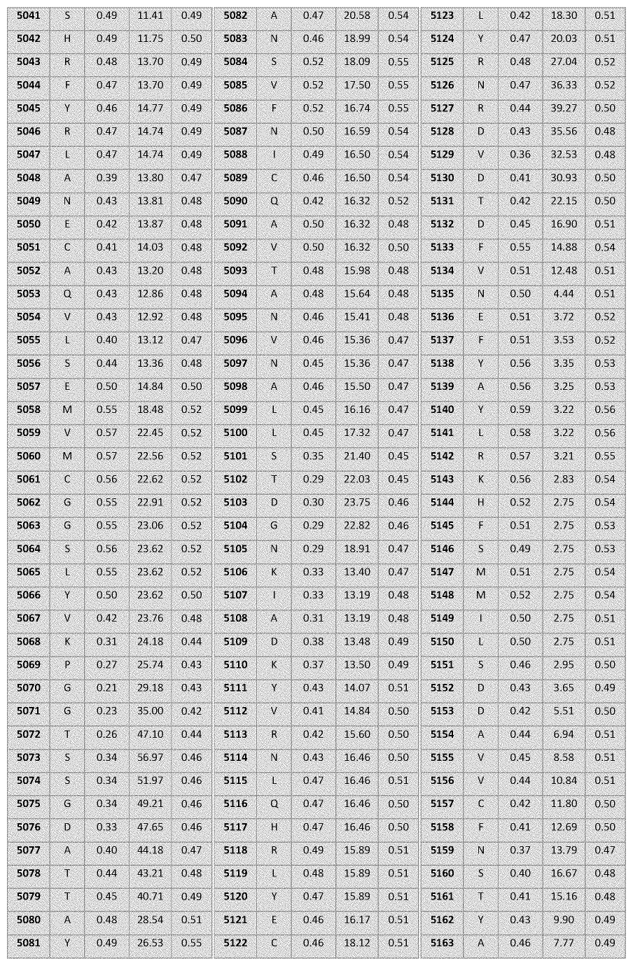

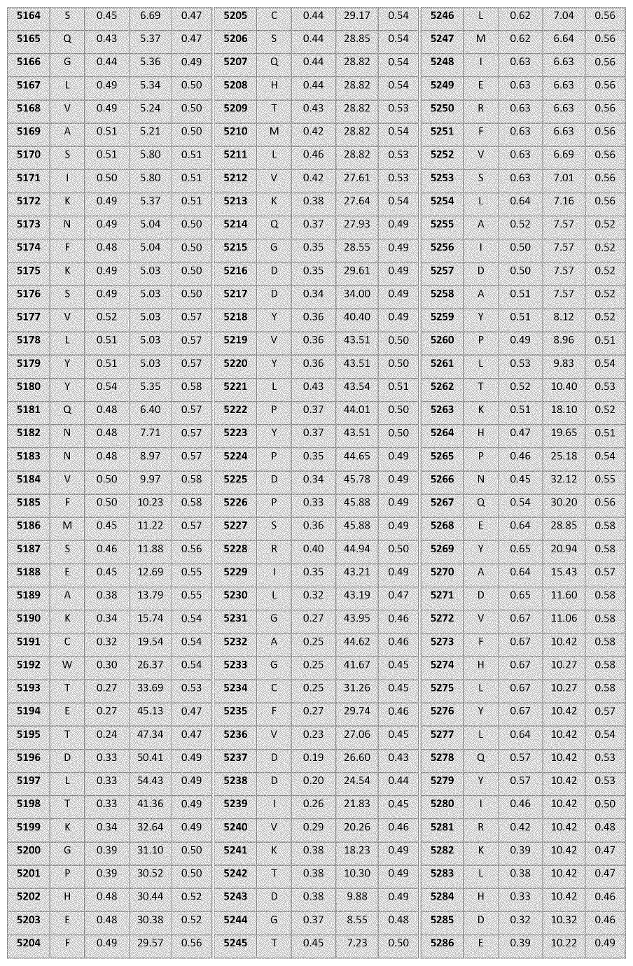

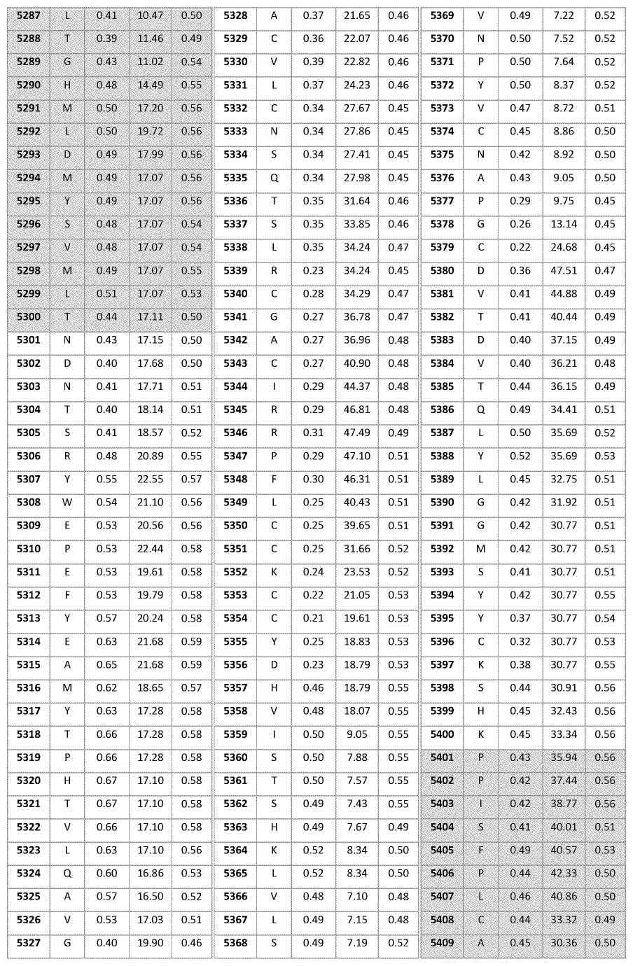

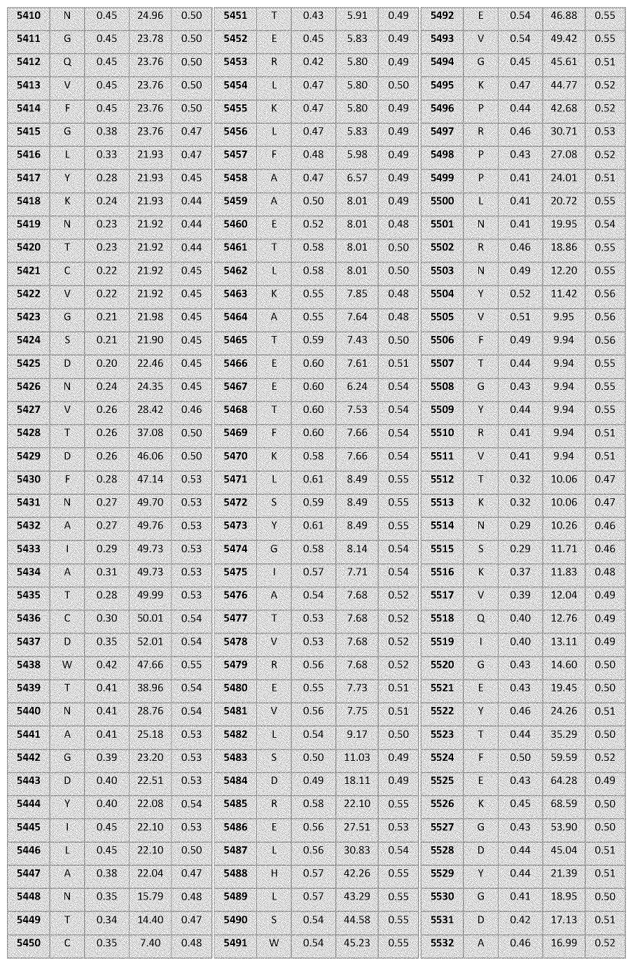

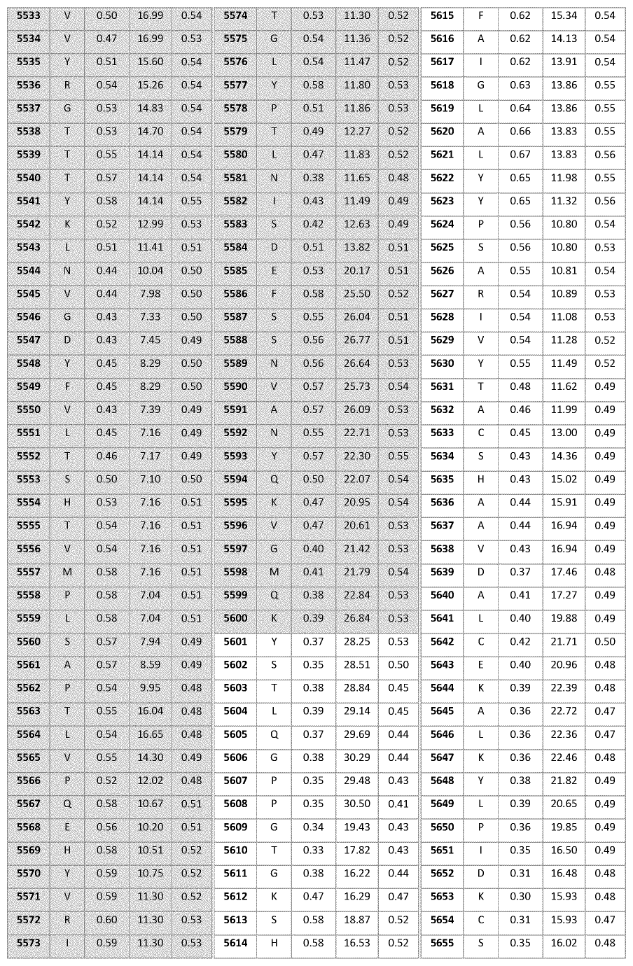

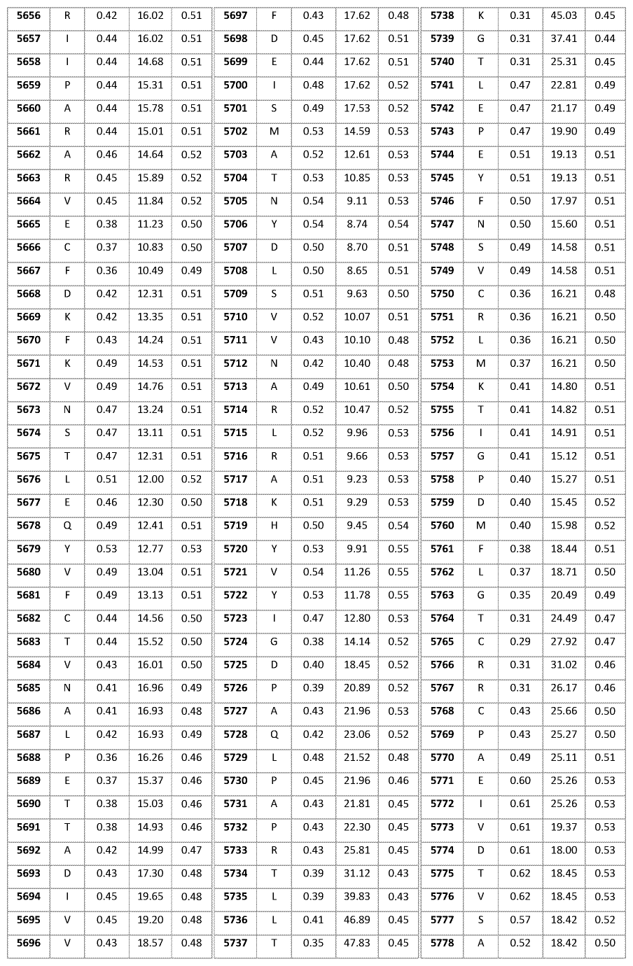

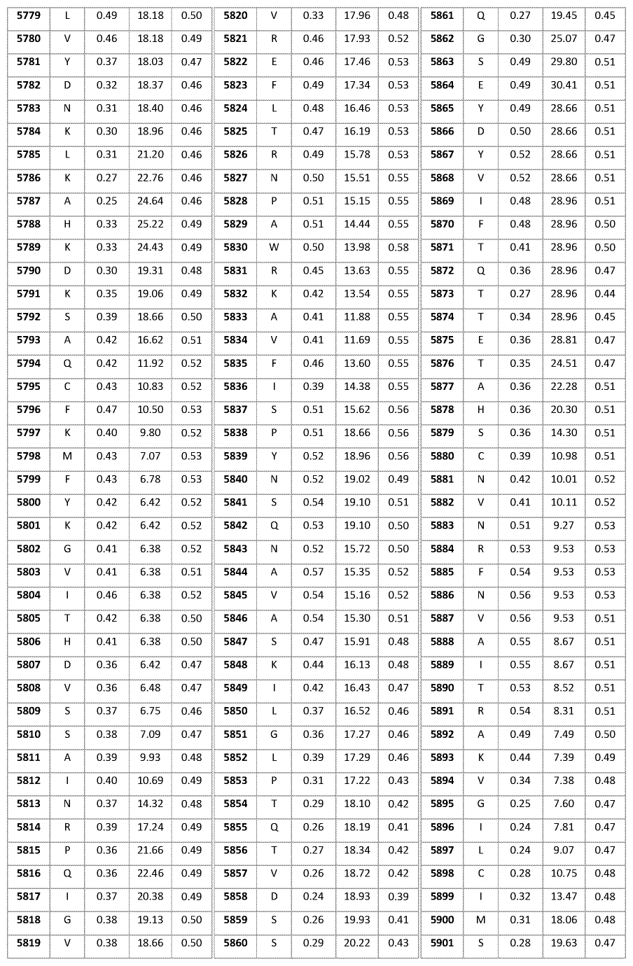

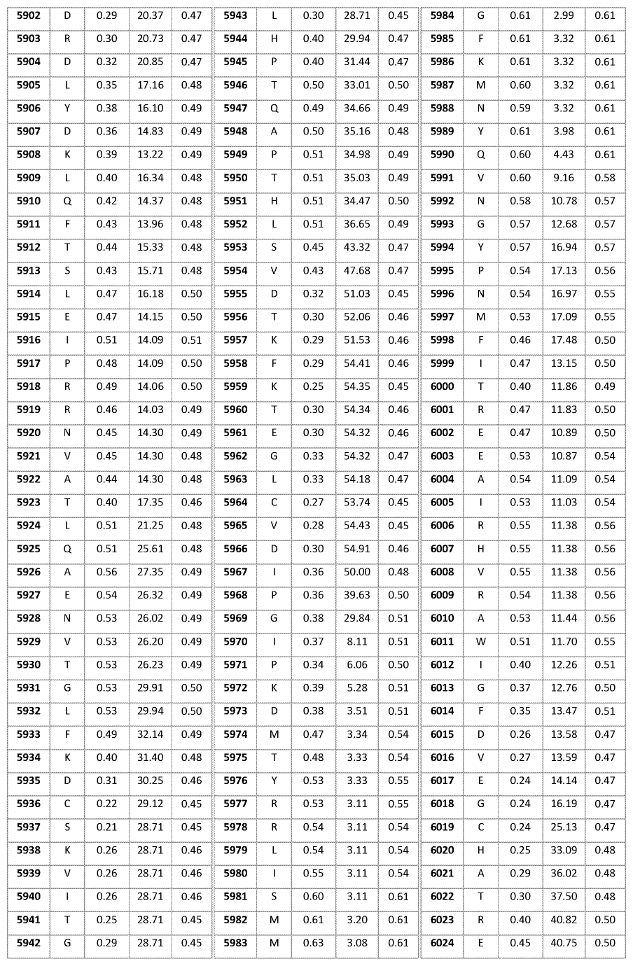

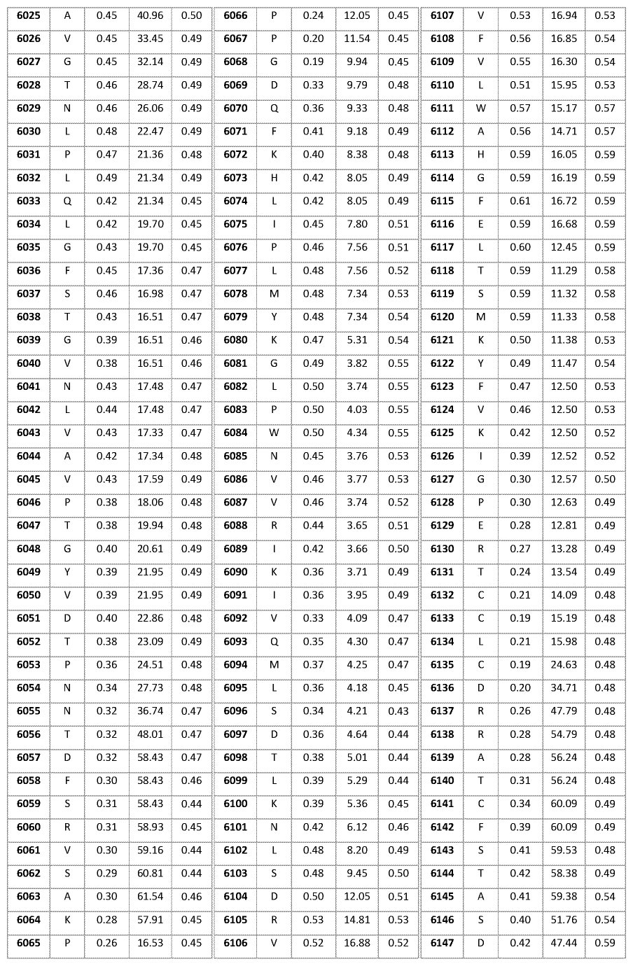

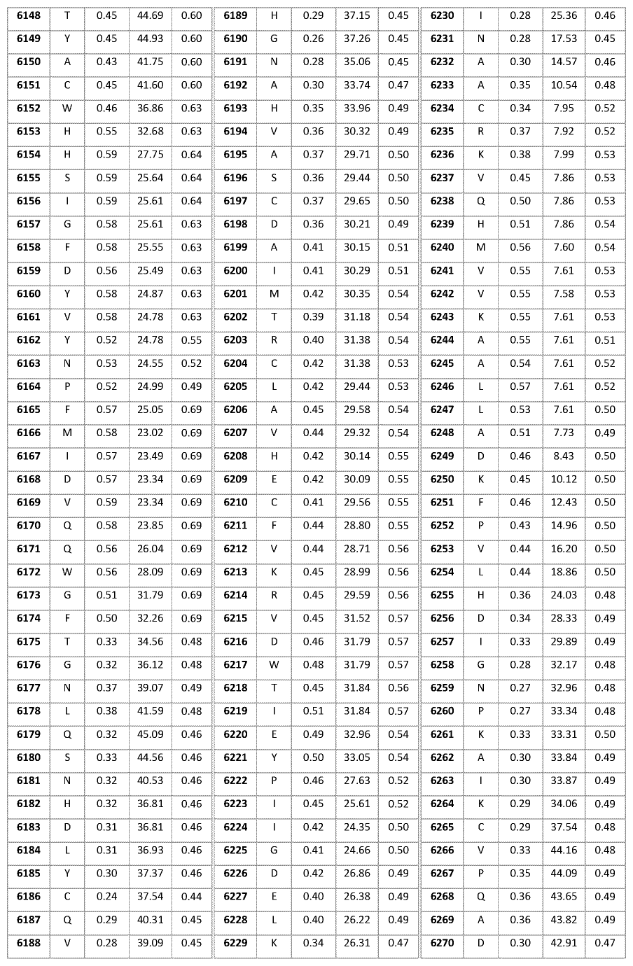

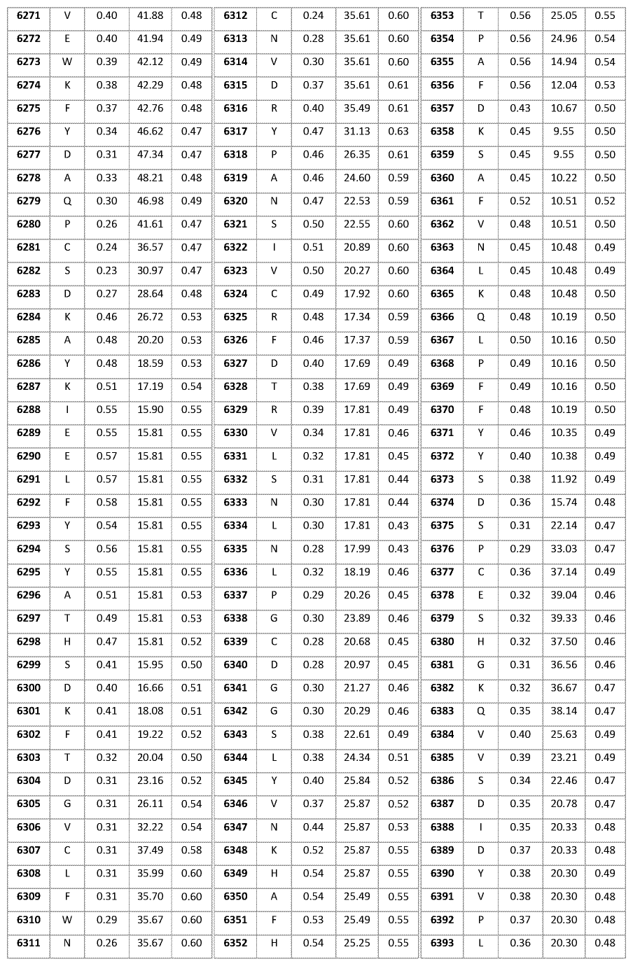

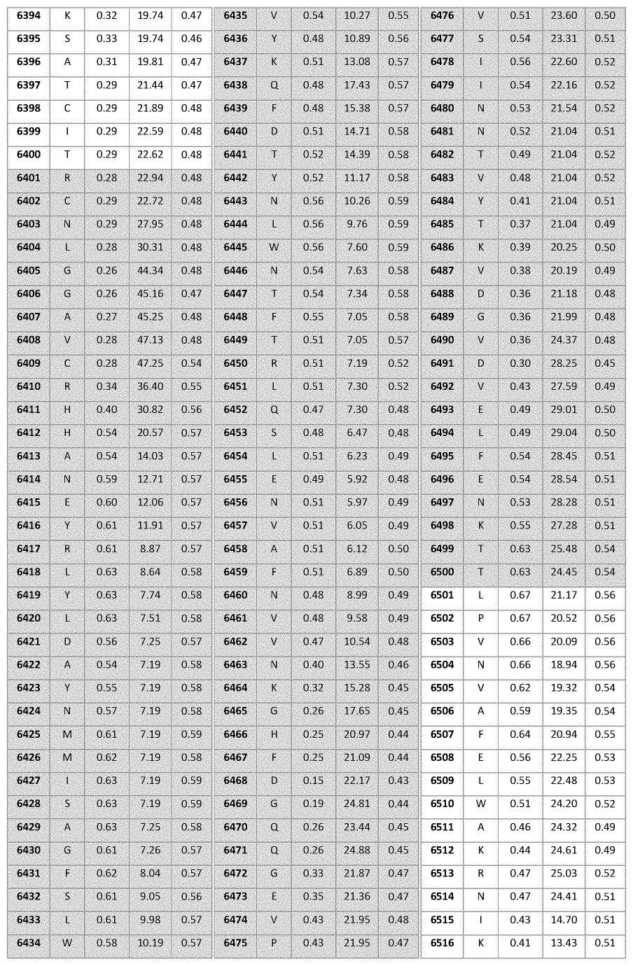

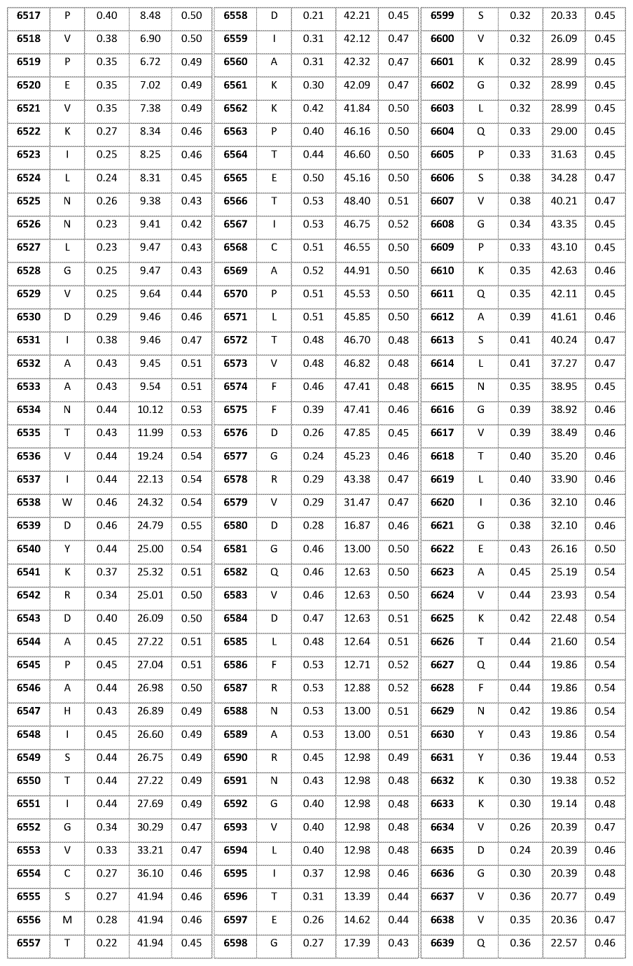

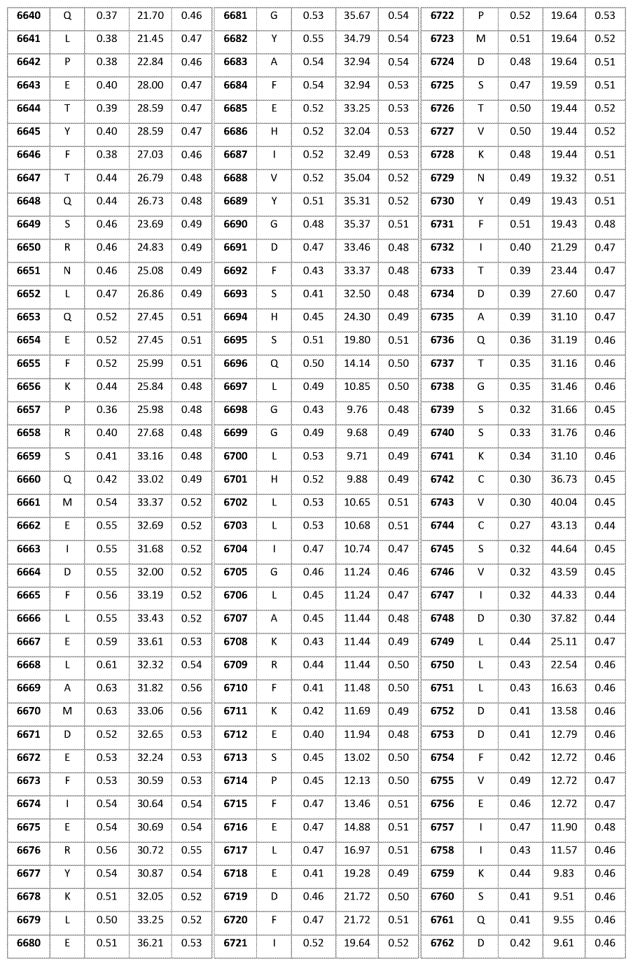

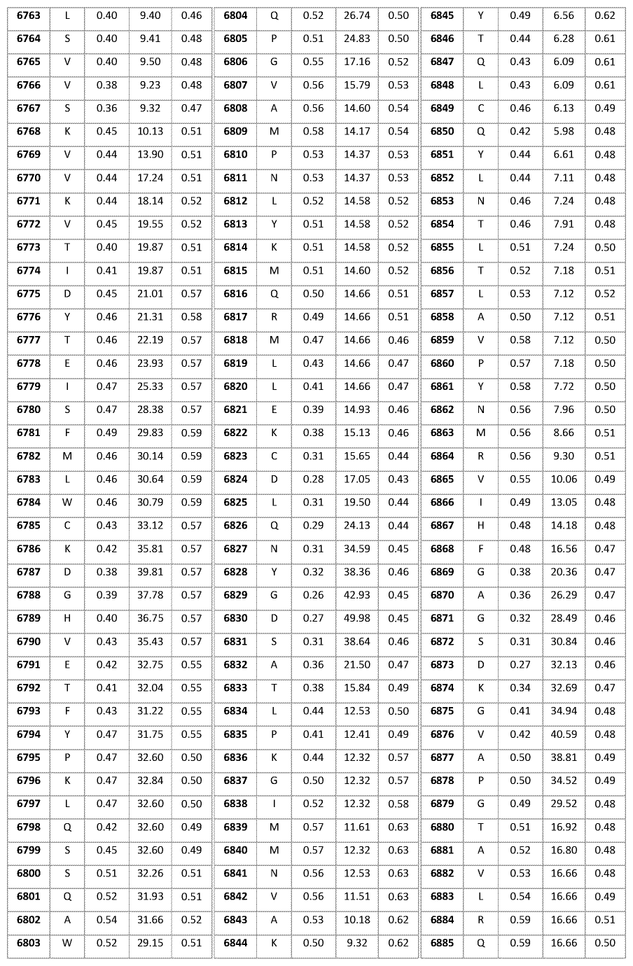

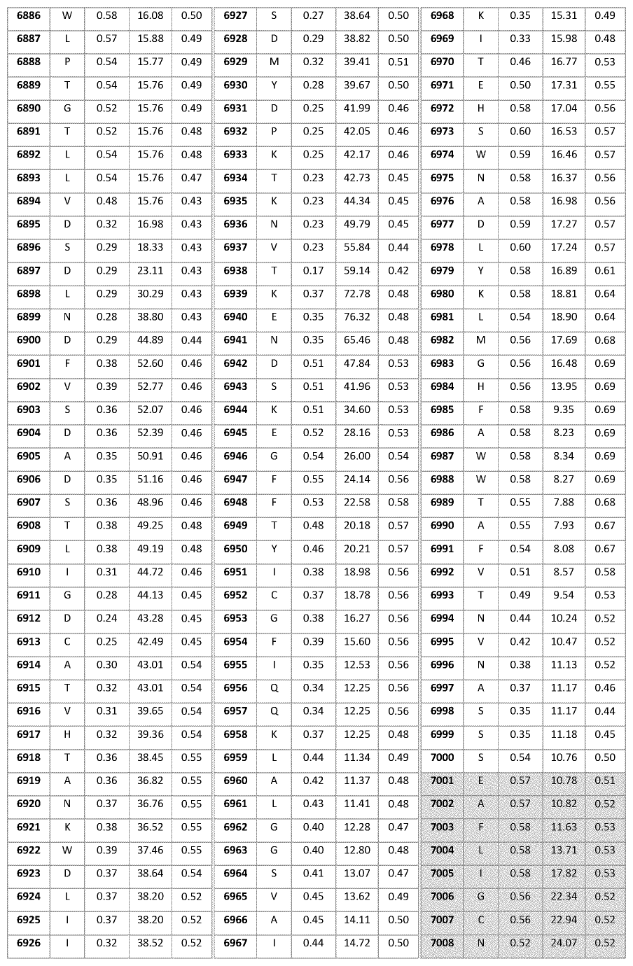

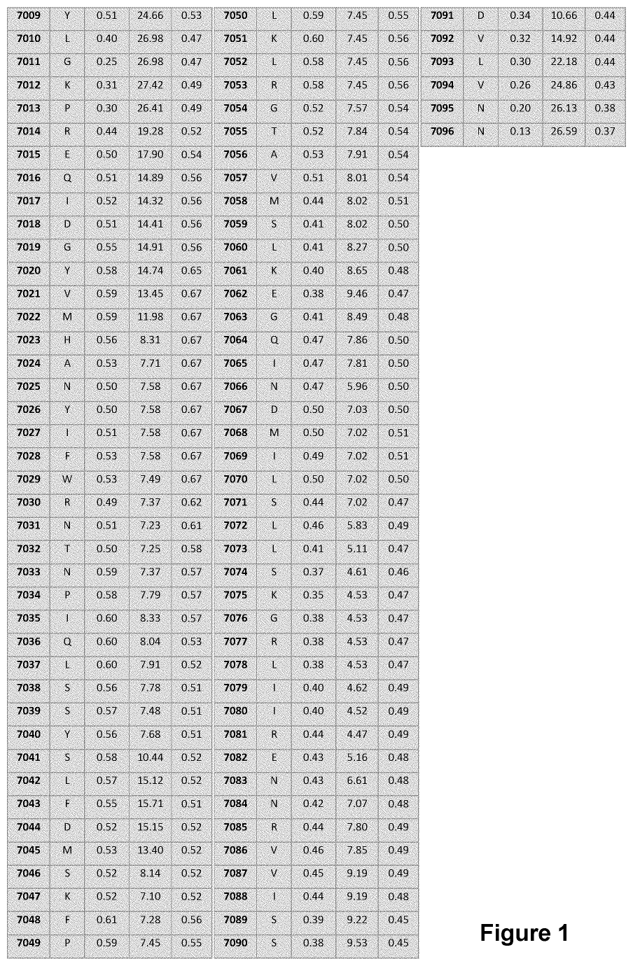

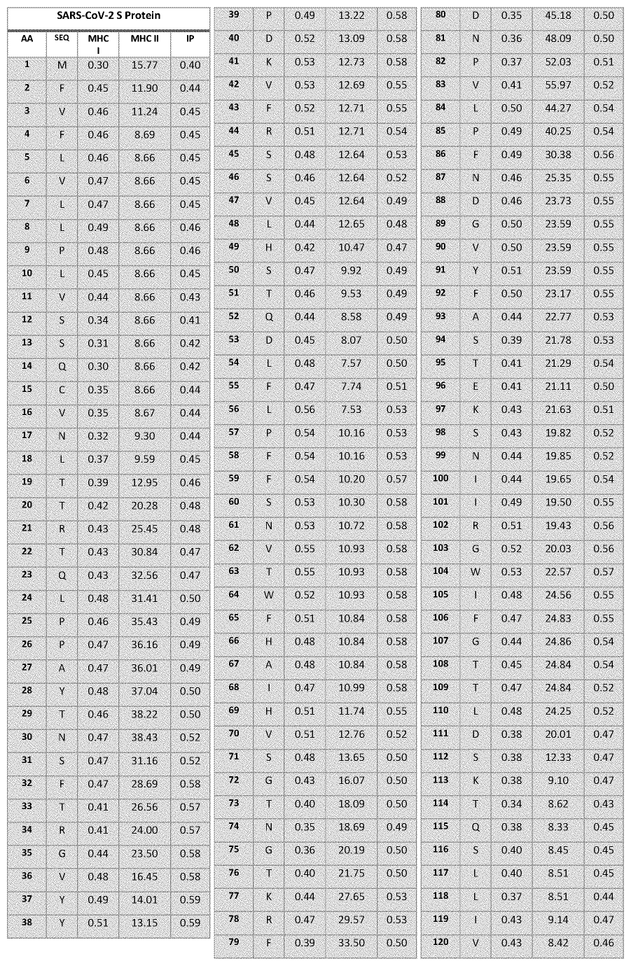

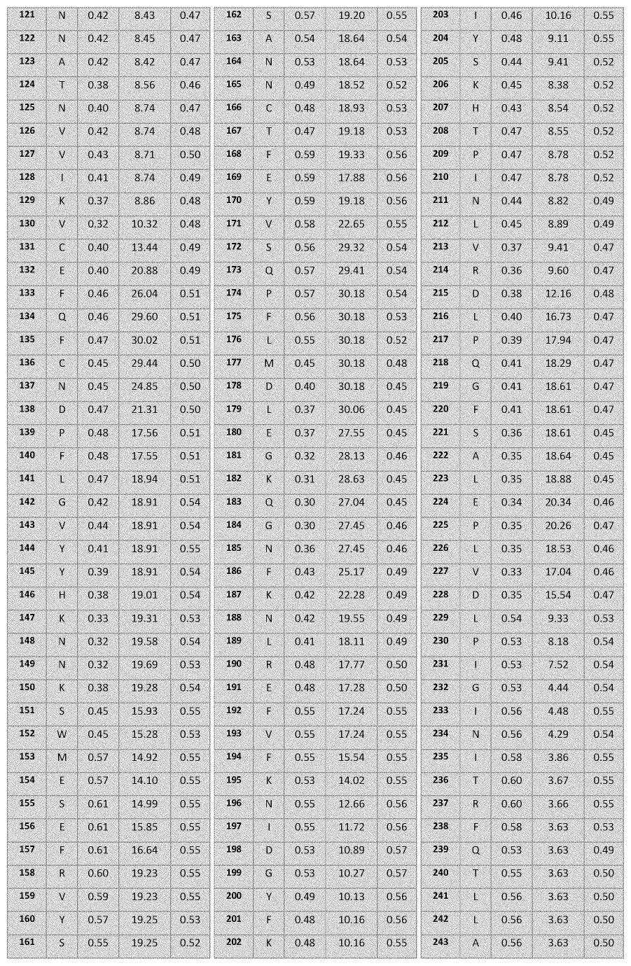

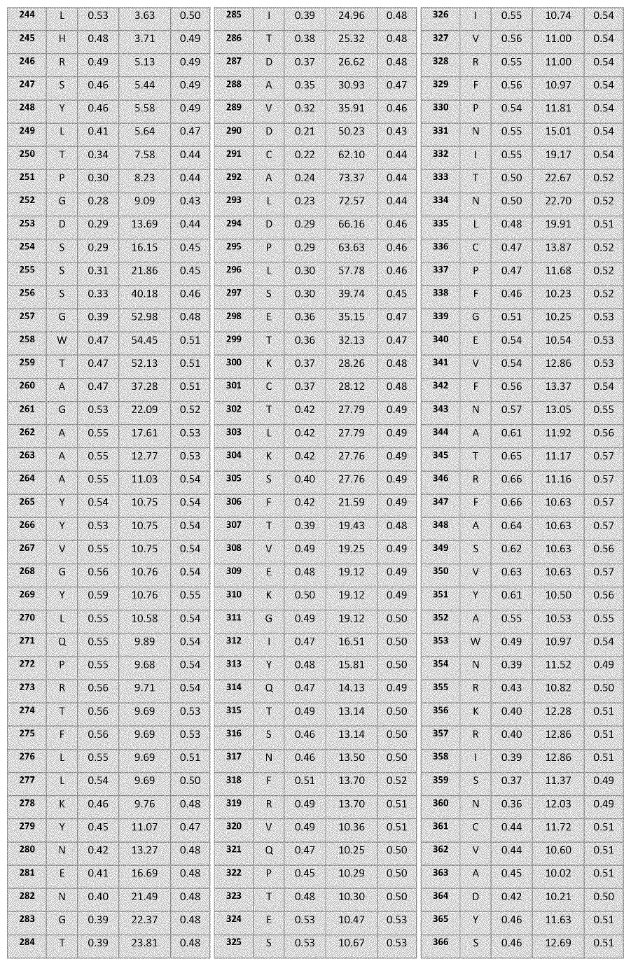

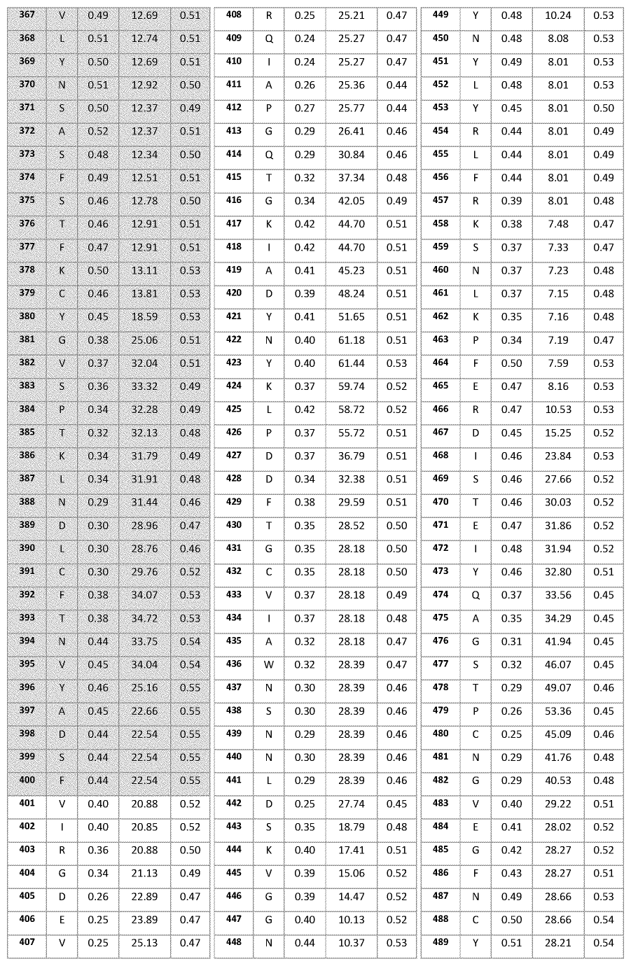

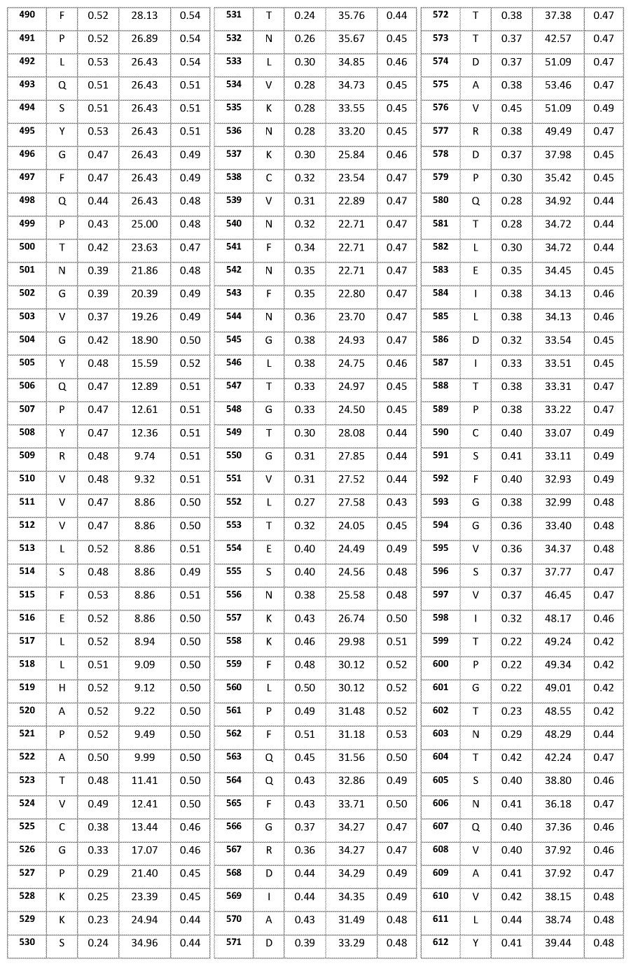

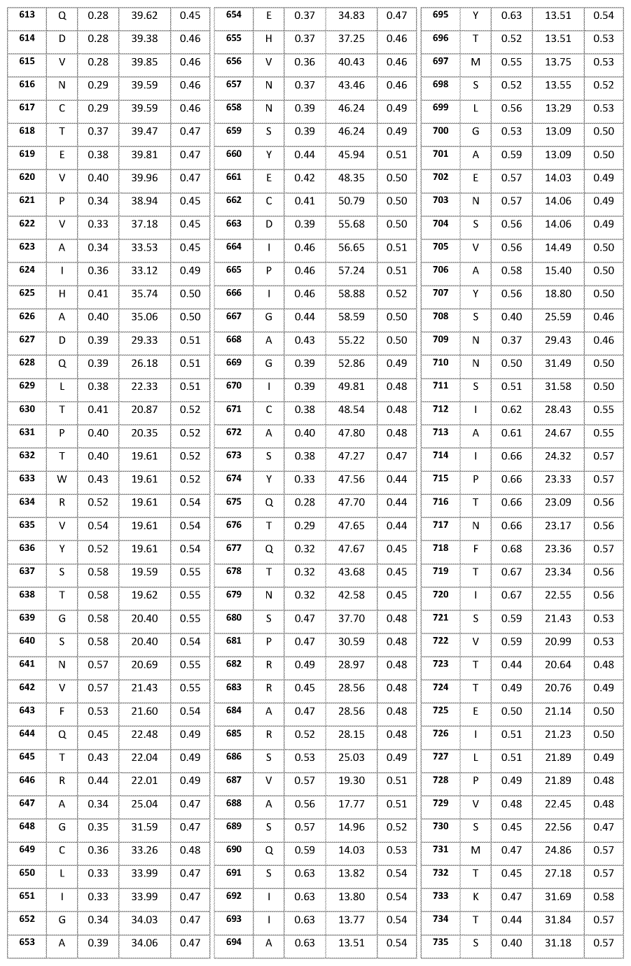

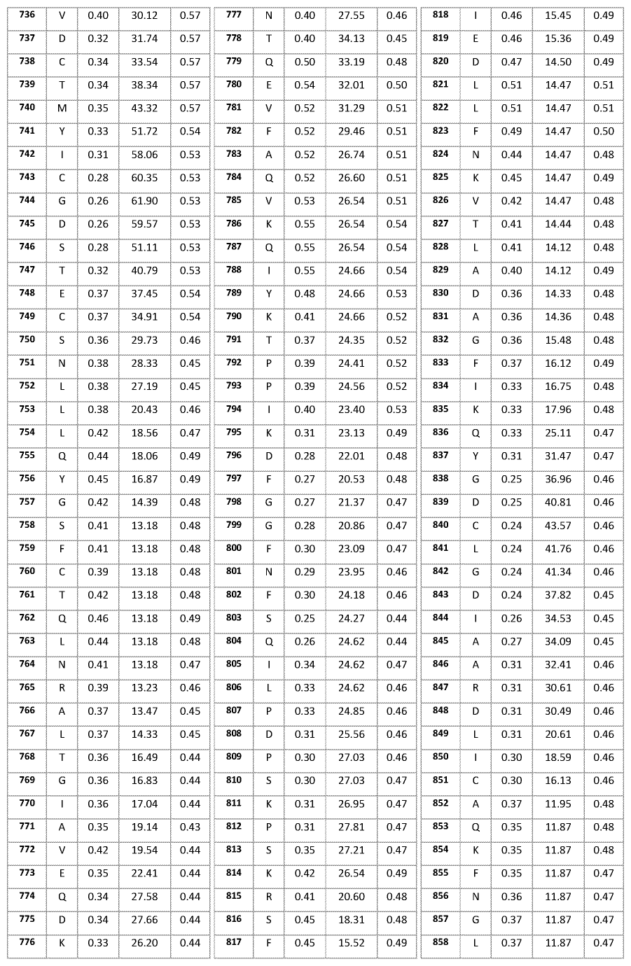

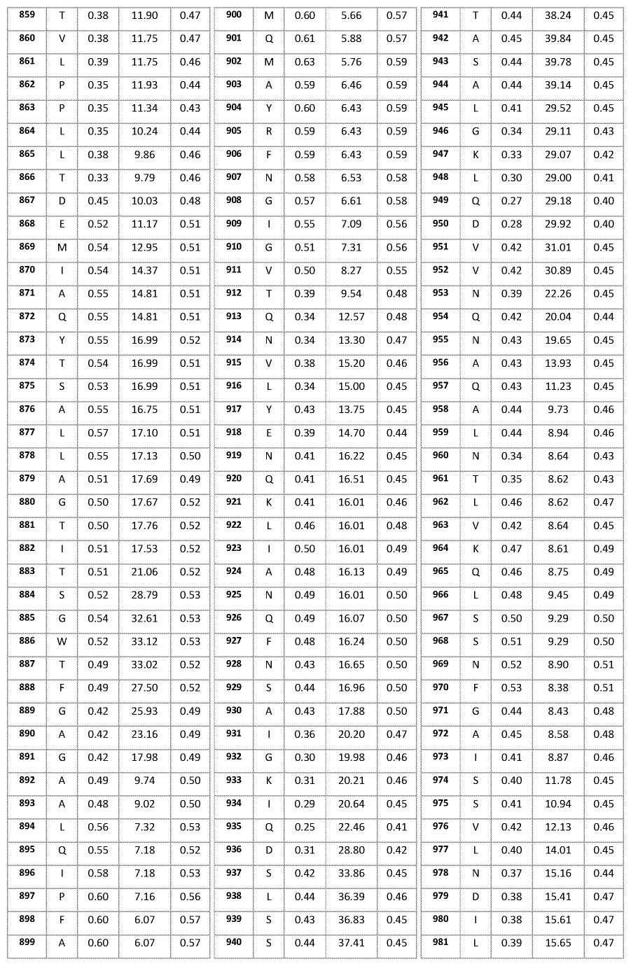

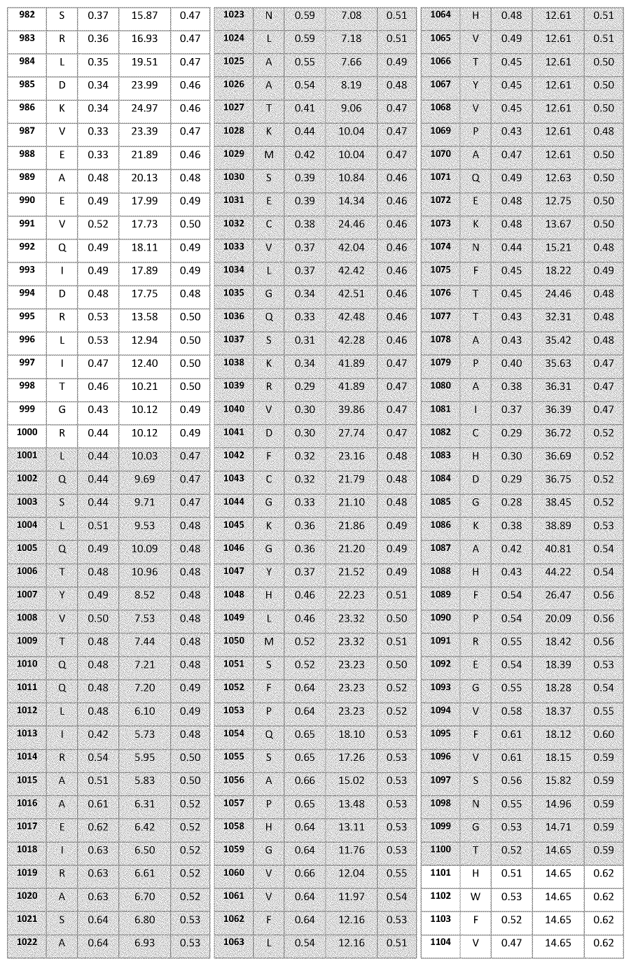

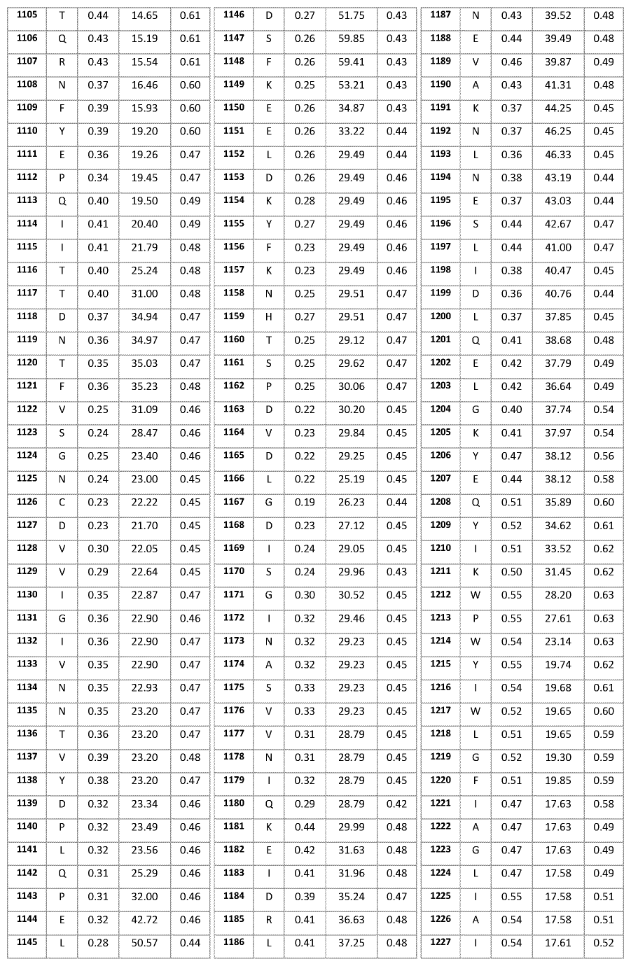

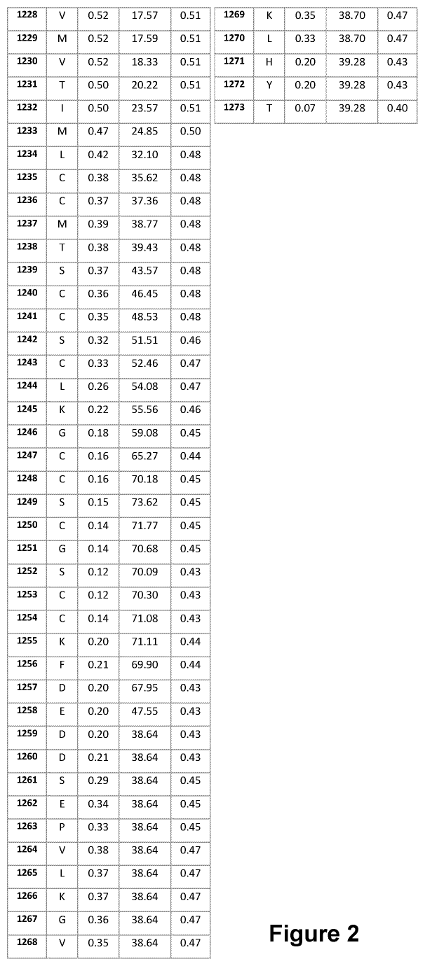

This A1-driven approach advantageously uses a statistical model to quantitatively analyse the predicted immunogenic potential of one or more epitopes—in other words the predicted ability of the one or more epitopes to instigate an immunogenic response-within an amino acid sub-sequence, across a set of different HLA types. The candidate regions (or “hotspots”) of the amino acid sequence that are identified by the quantitative statistical analysis may represent regions (or areas) of the one or more source proteins that are most likely to be viable vaccine targets and may be used in vaccine design and creation. These source proteins include each of the four structural proteins of SARS-CoV-2: the spike(S) protein, envelope (E) protein, membrane (M) protein, and nucleocapsid (N) protein, as shown in FIGS. 2, 4, 5 and 9, respectively. As well as said source proteins, the quantitative statistical analysis also utilised various open reading frames (ORFs) of the SARS-CoV-2 genome in its epitope mapping, as shown in FIGS. 1, 3, and 6-10.

It is envisaged that each of the hotspots identified herein may comprise one or more epitopes capable of stimulating an adaptive immune response through MHC Class I and/or MHC Class II. A candidate region may comprise a single epitope that is predicted to instigate an immunogenic response across a plurality of the HLA types. Such an epitope may be termed as “overlapping with” a number of HLA types. More typically however, a candidate region comprises a plurality of epitopes that, collectively, overlap with a large proportion of the analysed HLA types. For example, one epitope within a candidate region may overlap with n HLA types and a different epitope within the candidate region may overlap with m HLA types such that the candidate region is predicted to instigate an immunogenic response across the (m+n) HLA types.

The AI-driven approach comprised the step of assigning, for each of the set of HLA types, an antigen presentation (AP) score for each amino acid, wherein said score is indicative of the immunogenic potential of an epitope comprising that amino acid, for that HLA type. For a given HLA allele, the score allocated to an amino acid corresponds to the best score obtained by an epitope prediction overlapping with this amino acid. For Class I HLA alleles, 1 represents the best score, wherein the amino acid has a higher likelihood of being naturally presented on the cell surface, whereas a score closer to 0 represents a lower likelihood. For Class II HLA alleles, in contrast, the predictions are of percentile rank binding affinity scores wherein lower scores are best. With a range of possible output scores of 0 to 100 for Class II HLA alleles, a score of 0 represents the best score, with the highest binding affinity.

The predictions for Class I and Class II HLA types were performed using an antigen presentation and binding affinity prediction algorithm, as well as experimental data. Examples of publically available databases and tools that may be used for such predictions include the Immune Epitope Database (IEDB) (https://www.iedb.org/), the NetMHC prediction tool (http://www.cbs.dtu.dk/services/NetMHC/), the TepiTool prediction tool (http://tools.iedb.org/tepitool/), the NetChop prediction tool (http://www.cbs.dtu.dk/services/NetChop/) and the MHC-NP prediction tool (http://tools.immuneepitope.org/mhcnp/.). Other techniques are disclosed in WO2020/070307 and WO2017/186959.

Antigen presentation was predicted from a machine learning model that integrates in an ensemble machine learning layer information from several HLA binding predictors (trained on ic50 nm binding affinity data) and a plurality of different predictors of antigen processing (trained on mass spectrometry data).

Each of the identified epitopes was then preferably allocated a score based on the immunogenic potential predicted using the above techniques. Advantageously, the method not only identified candidate regions comprising epitopes that may bind to a HLA molecule, but also those CD8 epitopes that are naturally processed by a cell's antigen processing machinery, and presented on the surface of host infected cells.

The AP scores were assigned by the following protocol. Firstly, a plurality of epitopes were identified across the amino acid sequence, in a “moving window” of amino acids of fixed length. This was performed for each HLA type. For each of the identified first epitopes, a score was generated that is indicative of the immunogenic potential of that epitope, for the respective HLA type. A plurality of further epitopes were subsequently identified across the amino acid sequence, for each HLA type. Again, this was performed using a “moving window approach”. Each of the further epitopes were also assigned a score that was indicative of the immunogenic potential of that epitope, for the respective HLA type. Each amino acid was then assigned, for each HLA type, the score of the epitope that was predicted to have the best immunogenic potential of all the epitopes comprising that amino acid. Hence, for a particular HLA type, if epitope “A” and epitope “B” both comprised a particular amino acid “X”, the amino acid “X” would have been assigned the score of whichever epitope “A” or “B” is predicted to have the best immunogenic potential. In other words, for a given HLA type, the score allocated to an amino acid corresponds to the best score obtained by an epitope overlapping with this amino acid.

The AP score for each amino acid within a given source protein, or open reading frame, was averaged across HLA types, as shown in FIGS. 1-10. Two AP scores are given for each amino acids, wherein the first is the average AP score of that amino acid across 66 of the most common HLA-A and HLA-B alleles that correspond to MHC Class I, whilst the second is the average AP score of that same amino acid across 34 of the most common HLA-DR alleles that correspond to MHC Class II. In total, 100 of the most common human HLA-A, HLA-B and HLA-DR alleles across the globe were subjected to analysis.

The HLA types analysed may further be characterised into HLA types of the same or different human population groups. A population group may be an ethnic population group (e.g. Caucasian, Africa, Asian) or a geographical population group (e.g. Lombardy, Wuhan).

The AI-driven approach further involved the application of a Monte Carlo simulation, a statistical model that is used to identify regions of statistical significance. The input AP data of each amino acid for MHC Class I and MHC Class II across source proteins or ORFs was transformed into binary datasets such that for Class I values, a score of >0.7 was assigned a value of 1, whilst a score of ≤0.7 was assigned 0. For Class II binding affinity, values<10 were assigned a value of 1, whilst those ≥10 were assigned a value of 0. The Monte Carlo analysis identified statistically significant “bins”, “hotspots”, or regions of a protein, for a given selection of HLA types. In the case of the present invention, this selection of HLA types was the top 100 most common HLA-A, HLA-B and HLA-DR alleles in the human population, including 66 corresponding to MHC Class I and 34 to MHC Class II. The providing of the top 100 HLA alleles is not to be construed as a limitation to the epitopes of the present invention. The one or more epitopes of the present invention may, further to being able to interact with the top 100 HLA-A, HLA-B or HLA-DR alleles, also be able to stimulate a broad adaptive immune response across a plurality of HLA types including HLA-C, HLA-DQ and/or HLA-DP alleles.

The statistically significant hotspots were identified by a quantitative statistical analysis involving the designation of a region metric. The region metric for an amino acid sub-sequence hotspot is indicative of the predicted immunogenic potential of the one or more epitopes within the hotspot, across the tested set of HLA types. Thus, a “relatively better” region metric indicates that the one or more epitopes within that amino acid sub-sequence are collectively predicted to instigate an immunogenic response across a large proportion of the HLA types. A “relatively worse” region metric indicates that the one or more epitopes within that amino acid sub-sequence are not collectively predicted to instigate an immunogenic response across a large proportion of the HLA types in the analysis (for example epitope(s) within that amino acid sub-sequence are not predicted to instigate an immunogenic response at all, or only over a very few HLA types). Said region metrics were generated based on the AP scores for each amino acid within the respective hotspot amino acid sequence, across the set of selected HLA types.

Thus, by generating the region metrics based on the scores for the amino acids within the respective amino acid sub-sequence (which are in turn indicative of the immunogenic potential of a corresponding epitope), each region metric is indicative of the predicted immunogenic potential of the one or more epitopes within the respective amino acid sub-sequence, across the set of HLA types.

In the context of the present invention, the region metric is an average of the amino acid scores within the respective amino acid sub-region, across the set of 100 HLA types.

The Monte Carlo statistical model further identifies those hotspots that have a statistically significant region metric. In particular, the statistical model is applied to identify any region metric that is better than expected by chance. As would be understood by the skilled person, the significance threshold of such statistical modelling may be chosen accordingly, for example based on the perceived accuracy of the predicted immunogenic potential of the epitope(s). In the case of the present invention, a significance threshold was selected at a 5% false discovery rate (FDR), where those hotspots below 5% FDR represent regions that are most likely to contain presented epitopes based on the most frequent HLA alleles in the human population. The FDR procedure used within the present invention was the Benjamin-Hochberg procedure.

The application of the Monte Carlo simulation allowed for the estimation of a p-value for each of the generated region metrics. These estimated p-values were then used to identify the statistically significant amino acid sub-sequence hotspots and, consequently, the candidate regions (hotspots). The null model for this statistical modelling is typically defined as the generative model of the set of amino acid scores, for each HLA type, if they were to be generated by chance.

The set of amino acid scores for a particular HLA type may be referred to as an “HLA track”. The Monte Carlo simulation was used to iteratively produce a set of 100 HLA tracks and a plurality of associated simulated region metrics, from which the p-value—and hence the statistical significance—of each region metric was estimated.

The arrangement of the amino acid scores for each HLA type (arrangement of each HLA track) into a plurality of epitope segments and epitope gaps reflects whether the amino acid was part of an epitope predicted to have a good immunogenic potential or not, based on its assigned score. Thus, an epitope segment is a consecutive sequence of (typically at least 8) scores assigned to amino acids within an epitope predicted to have a good immunogenic potential, and an epitope gap is one or more consecutive scores assigned to amino acids that are not part of such epitopes. By iteratively randomizing the epitope segments and epitope gaps rather than individual amino acid scores, the null model more faithfully reflects the methodology behind the region metrics, thereby providing a more reliable result.

As a further step in the identification of suitable epitopes for the present invention, the outputted average AP scores were used as input to compute “immune presentation” (IP) across the epitope map. The IP score is representative of HLA-presented peptides that are likely to be recognised by circulating T cells in the periphery, i.e. T cells that have not been deleted or anergised, and thus are most likely to be immunogenic. The degree of immunogenicity would prove beneficial in the context of the present invention, as would be appreciated by the skilled person.

The IP score also penalizes those peptides that have degrees of “similarity to self” against the human proteome, and awards peptides that have “distance from self”. The resulting IP score identifies therefore those T cell epitopes that are not tolerized, and therefore most likely to induce unwanted auto immune responses. The concept of tolerance, or central tolerance, refers to the negative selection process of eliminating any developing T or B cells that are reactive to self, ensuring that the immune system does not attack self-peptides. T cells must have the ability to recognise self MHC molecules with bound non-self peptides. During negative selection, T cells are tested for their affinity to self, wherein if they bind a self peptide, they are signalled to apoptose.

T cell epitopes that have a high degree of similarity to self may induce autoimmune pathology in a processed named “molecular mimicry”. Such autoimmune pathologies are involved with the generation of an immune response against self-tissue and cells, which may include rapid polyclonal activation of B or T cells and/or a detrimental release of cytokines and alteration of macrophage function (Karlsen & Dyrberg 1998, Seminars in Immunology (1): 25-34).

In the present invention, an IP score of at least 0.5 is considered immunogenic, and could represent a threshold for inclusion within the vaccine composition. The threshold value represents a safe margin of considerable confidence, wherein IP values of above said threshold are considered appropriately representative of “further from self”, whilst values below are considered appropriately representative of “similar to self”. It is further envisaged that as an alternative utilisation of the IP score, exclusion may be carried out on an epitope basis, wherein those epitopes that have an average IP score of below 0.5 may be discarded from the selection of epitopes included within the vaccine composition.

The IP score of each amino acid within the analysed proteins and open reading frames is listed in FIGS. 1-10.

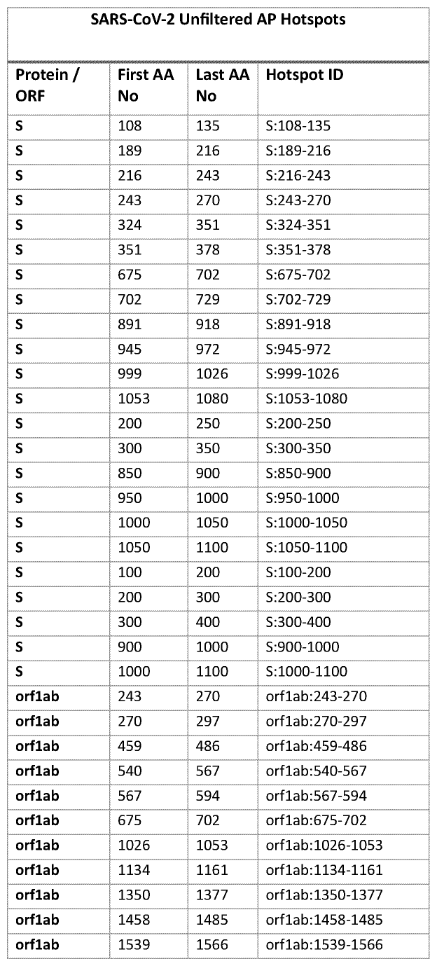

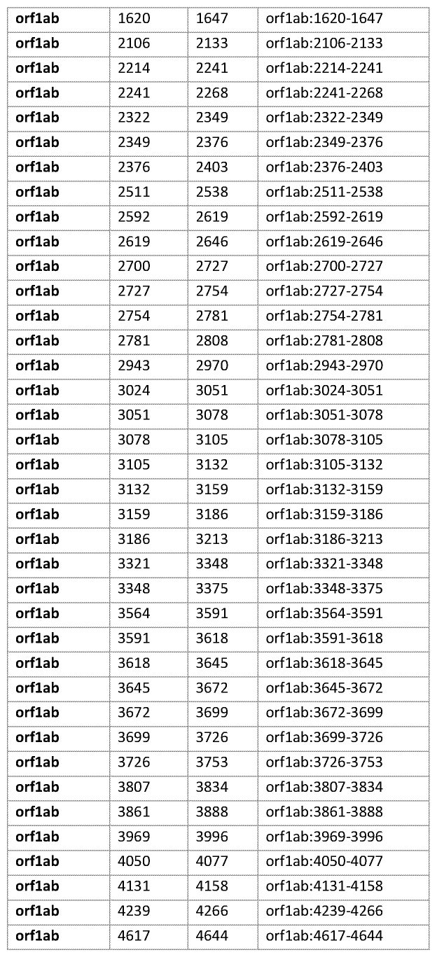

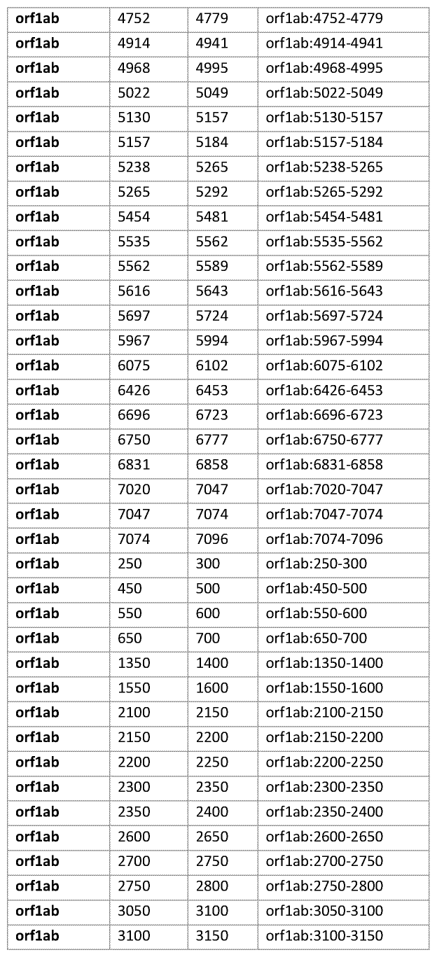

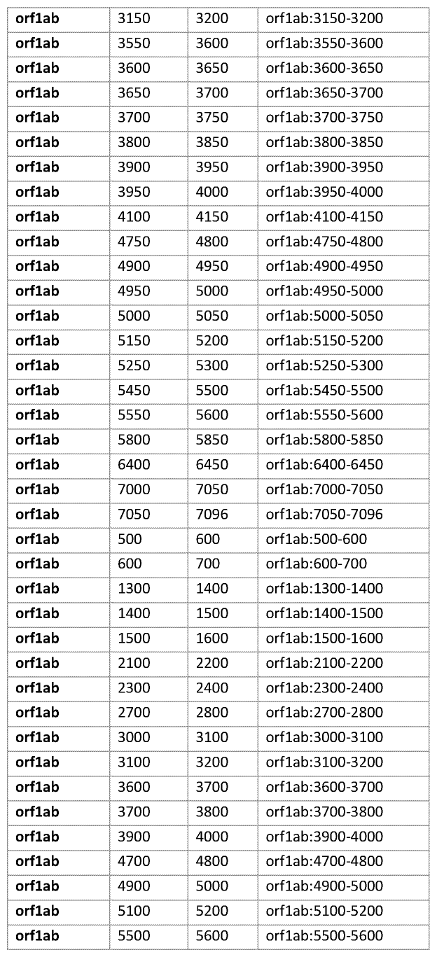

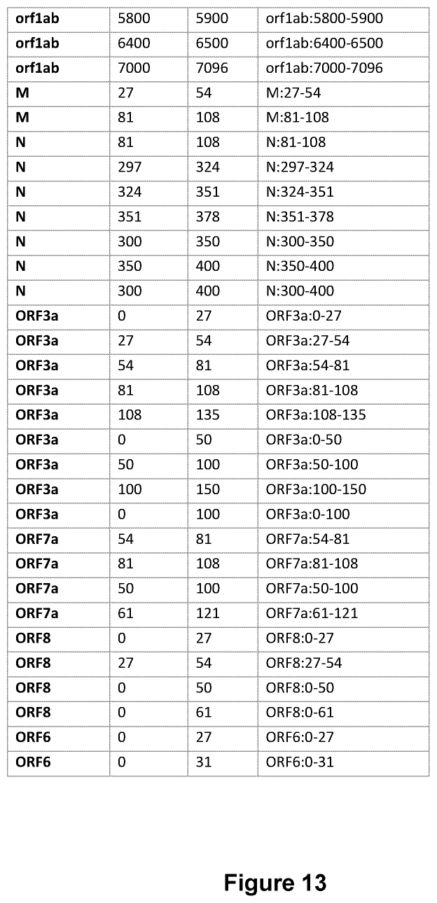

It is envisaged that the coronavirus vaccine composition of the present invention comprises one or more epitopes found within any one or more of the hotspots, including SEQ ID NOs: 1-30 within Table 1, as well as comprised within FIGS. 13-18, wherein said epitopes are at least 8 amino acids in length, and wherein said epitopes meet a particular threshold of a mean antigen presentation (AP) cut off value and an IP score of at least 0.5. Said mean AP cut off value is the value, averaged across all amino acids within an epitope, for which said epitope is considered able to stimulate a broad adaptive immune response across a plurality of HLA types, for either MHC Class I and/or MHC Class II immunogenicity.

For the sake of avoiding confusion, the term “antigen presentation (AP) value” may be used to mean binding affinity or percentile ranking, and the terms shall be used interchangeably. As such, reference to a mean “AP cut off value” in the context of MHC Class II, is to be construed as the mean binding affinity or mean percentile ranking of the relevant epitopes.

In some embodiments of the present invention, the mean AP cut-off value may be ≥0.4 for MHC Class I, and/or ≤13 for MHC Class II. In a preferred embodiment, the mean AP cut-off value may be ≥0.5 for MHC Class I, and/or ≤10 for MHC Class II.

It is envisaged that the coronavirus vaccine composition of the present invention may comprise any number of epitopes as would be suitable for use within a vaccine composition. In some embodiments, the composition comprises at least epitopes. In a preferred embodiment, the composition comprises between 5 and 10 epitopes. In a yet further preferred embodiment, the composition comprises between 5 and 20 epitopes, most preferably 10-12 epitopes. As disclosed herein the vaccine composition may be prepared by selecting individual epitopes as defined herein, or the epitopes may be contained in the hotspot regions which are prepared as part of the vaccine composition.

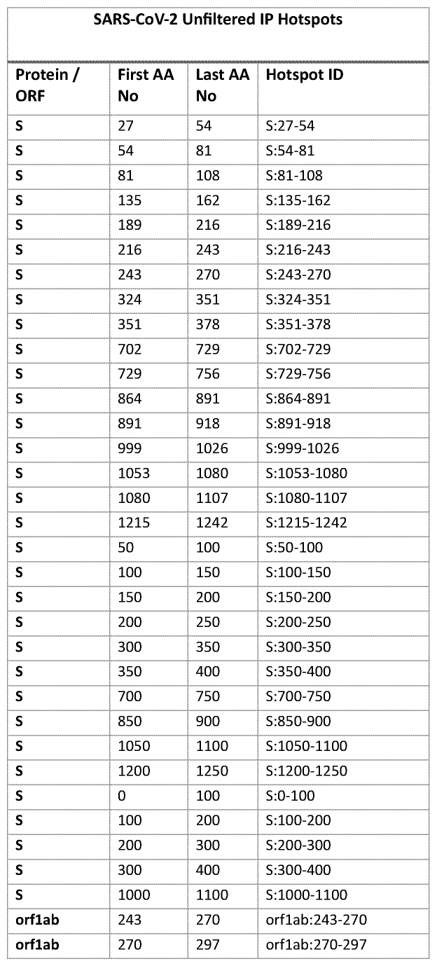

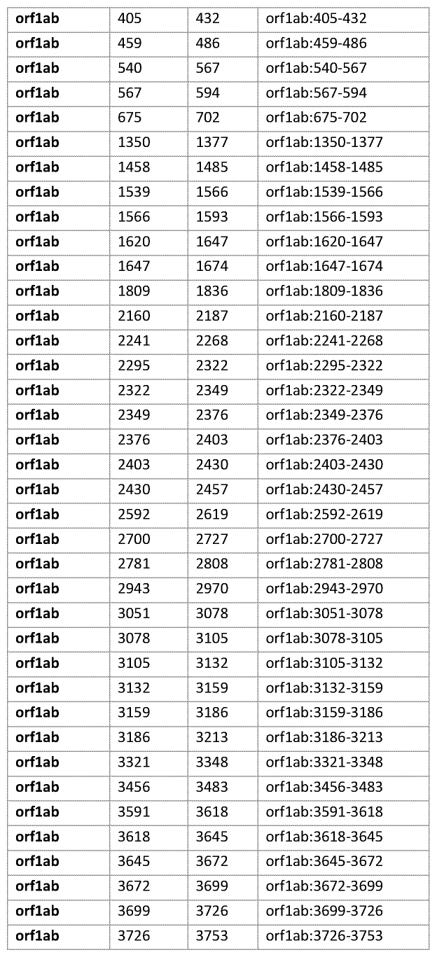

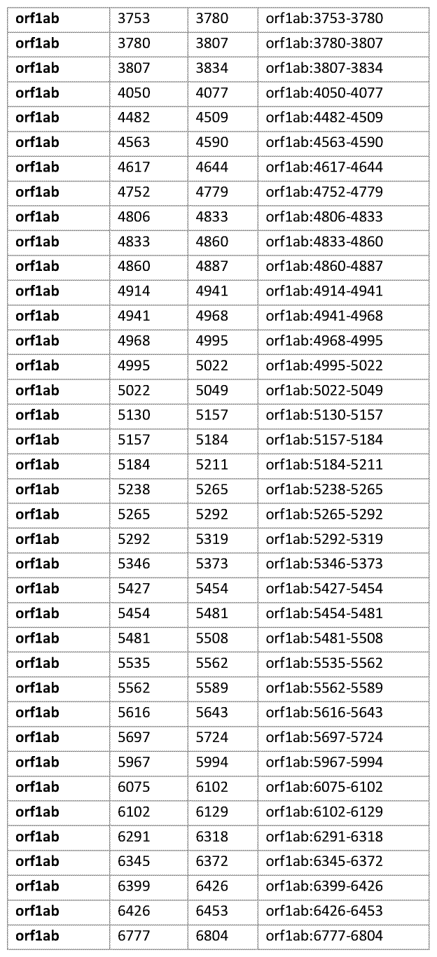

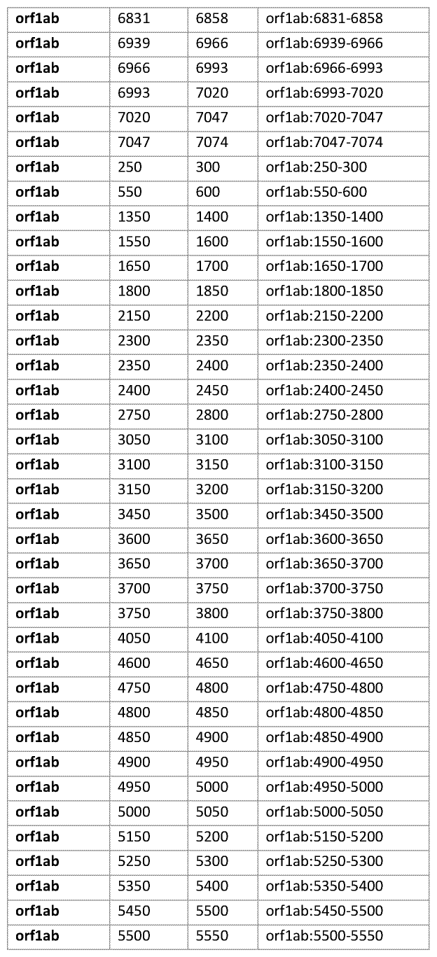

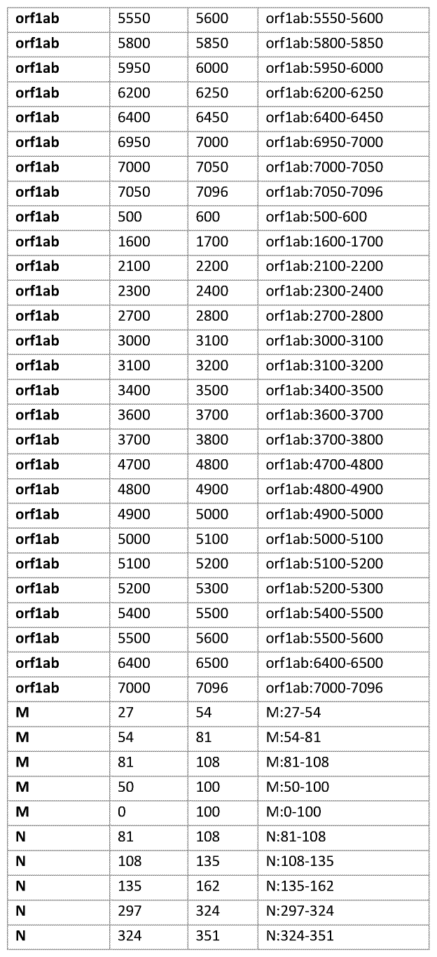

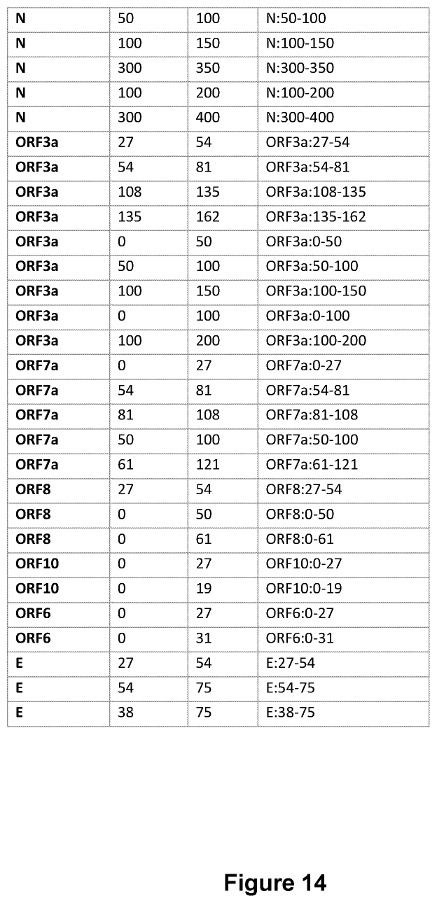

A selection of defined hotspots have been listed in FIGS. 13 and 14, representing the “unfiltered” epitopes with their corresponding AP scores, and IP scores, respectively. This selection of defined hotspots can be further filtered to preferred embodiments classified under AP and IP scores, as listed in FIGS. 15 and 16 respectively. The filtering refers to a process of identifying similarity to self, as described previously, as well as preferentially selecting those hotspots that may be found within particularly conserved regions of the viral proteome. As such, this step would advantageously comprise filtering the one or more candidate regions so as to select one or more candidate regions in conserved areas of the one or more proteins (i.e. areas less likely to present mutations).

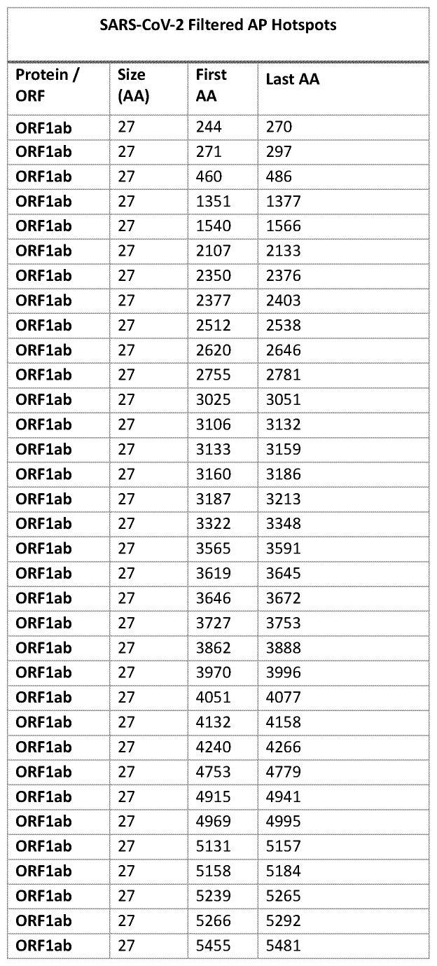

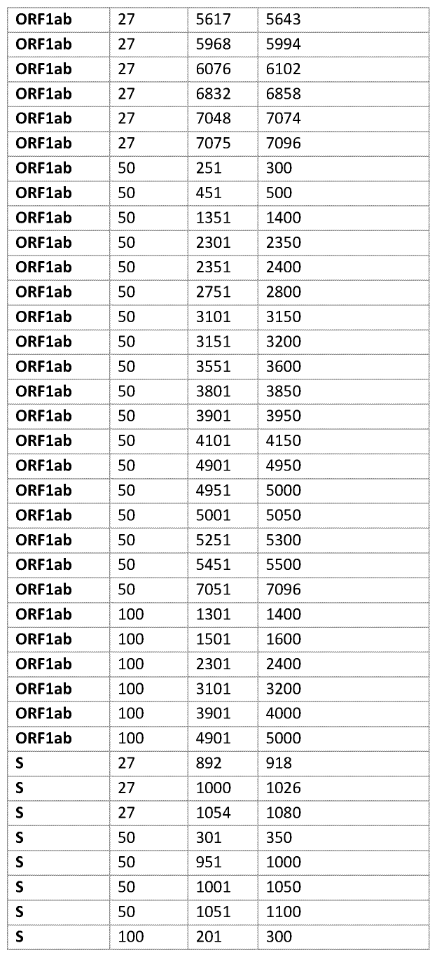

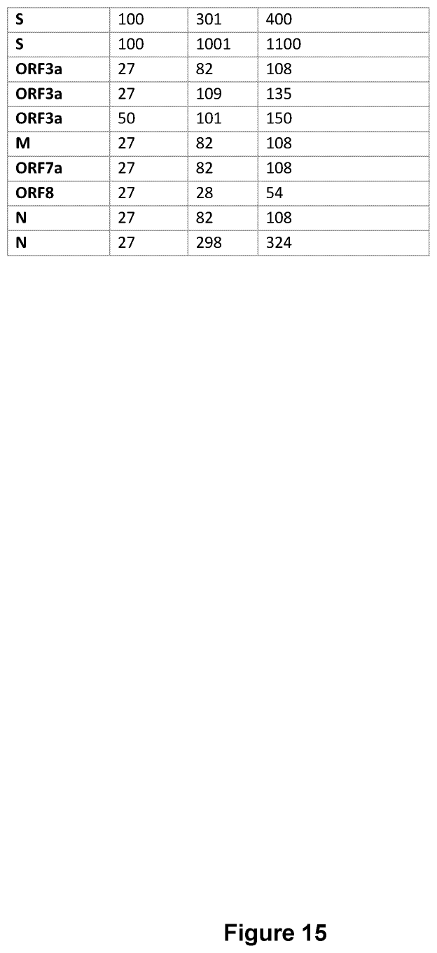

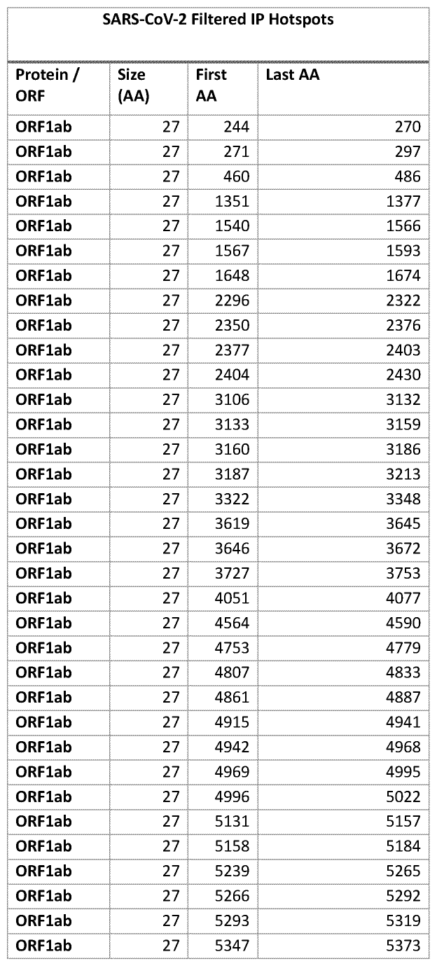

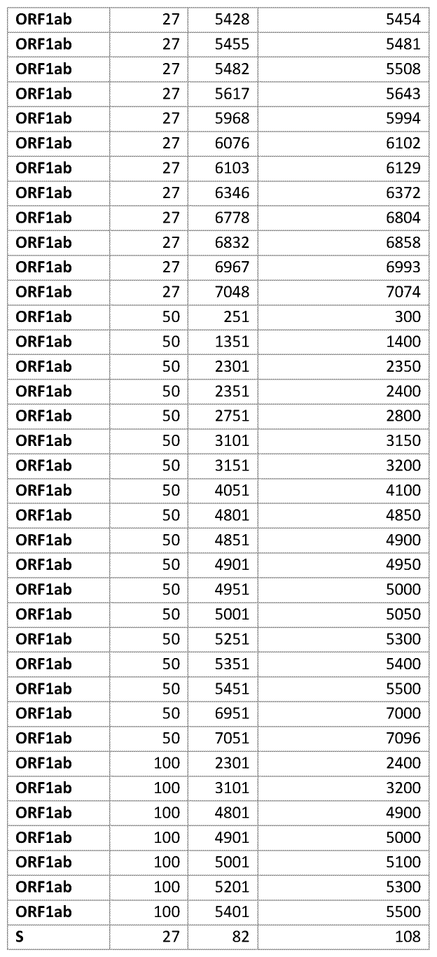

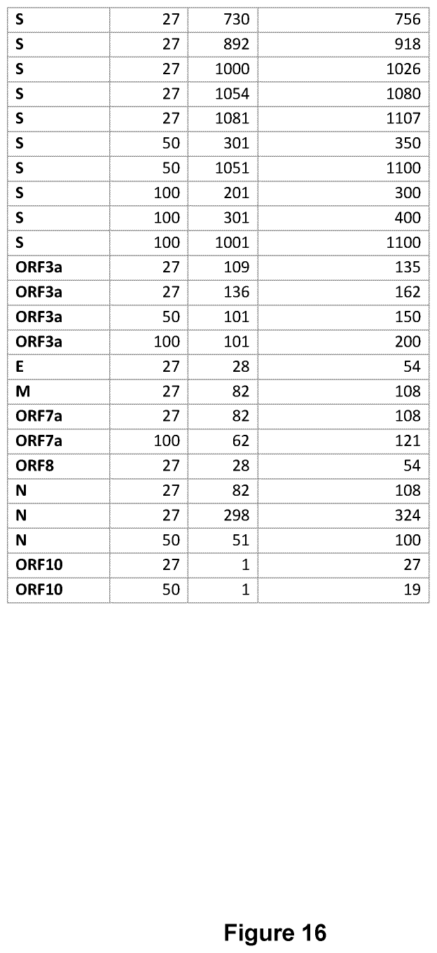

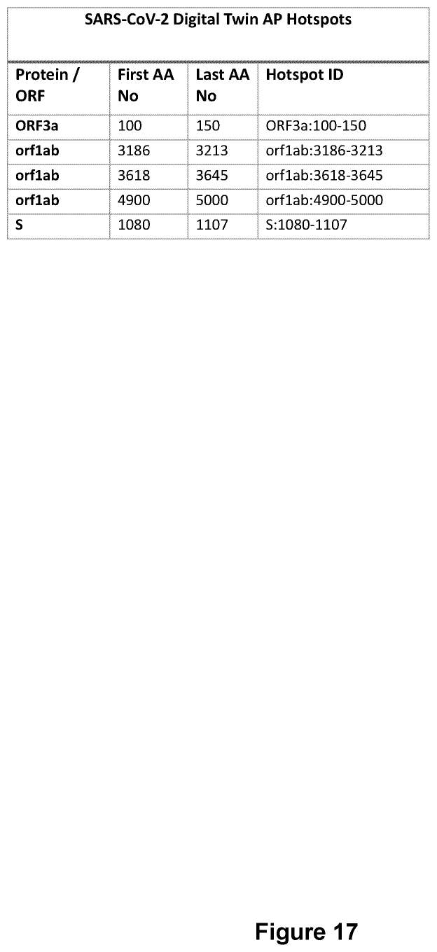

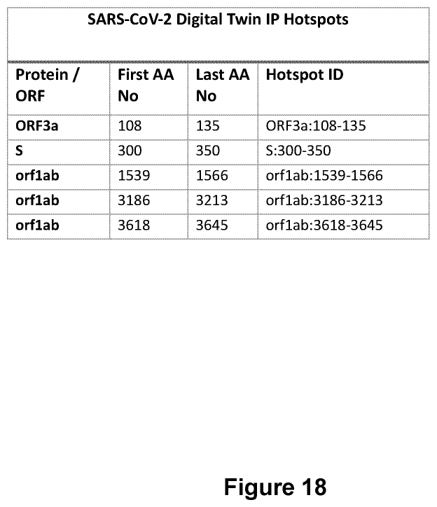

Conserved regions may be identified using techniques known in the art. In yet a further approach of refining the hotspot selection, a digital twin analysis—as explained in Example 5—was carried out: a method and system for selecting a small set of candidate peptides, or hotspot regions, for inclusion in a vaccine such that the likelihood that every member of a population has a positive response to the vaccine is maximised. This refined selection of most preferred hotspots is shown in FIG. 17, in the context of AP values, and FIG. 18, in the context for IP values.

Thus, in some embodiments of the invention, the composition may comprise one or more epitopes found within FIG. 13 or 14. In a preferred embodiment, the one or more epitopes may be found within FIG. 15 or 16. In yet a further preferred embodiment, the one or more epitopes may be found within FIG. 17 or 18.

As noted within the description of the figures, a variety hotspot regions identified in FIGS. 1-10 have been highlighted via grey scaling for the ease of the skilled reader, wherein said hotspot regions are unfiltered and may be around 100 amino acids in length. Such highlighted hotspots are not exhaustive of the total identified hotspots of the invention, and are merely an indication of several optional embodiments.

In some embodiments, the composition may comprise one or more epitopes found within any one or more of FIGS. 13-18 and/or Table 1.

In a preferred embodiment, the one or more epitopes may be any one or more of the epitopes listed in Table 1 and/or FIG. 17. In a further preferred embodiment, the one or more epitopes may be any one or more of the epitopes listed in Table 1 and/or FIG. 18.

It is envisaged that the composition of the present invention may comprise an immunogenic portion of the coronavirus, wherein the term “immunogenic portion” refers to one or more epitopes found within any one or more of FIGS. 1-10, or a polynucleotide encoding said epitope. Each epitope within said immunogenic portion must be at least 8 amino acids in length, and each epitope considered able to stimulate a broad adaptive immune response across a plurality of HLA types, for either MHC Class I and/or MHC Class II immunogenicity.

In some embodiments, the size of said immunogenic portion may have, or express, an upper limit of 450 amino acids in length, preferably 300 amino acids in length. In other embodiments, the upper limit may be 200 amino acids in length. In a further embodiment, the upper limit may be 50 amino acids. In yet another further embodiment, the upper limit may be 30 amino acids in length. Accordingly, the immunogenic portion may consist of the complete (discrete) sequence defined herein as a hotspot, or fragments thereof that comprise at least one of the epitopes defined herein.

It is envisaged that such an immunogenic portion for use in the composition of the present invention would be recombinant in nature, wherein recombinant refers to the artificial and/or modified characteristic of said immunogenic portion, which may be produced through genetic recombination means. As such, it is envisaged that the immunogenic portion may be a discrete, non-functional, recombinant fragment of a protein, such as that of SARS-CoV-2 spike(S) protein or SARS-CoV-2 membrane (M) protein, wherein said non-functional, recombinant fragment includes one or more of the epitopes of at least 8 amino acids in length, capable of stimulating a broad adaptive immune response across a plurality of HLA types, as described in the present invention.

The vaccine may comprise multiple discrete immunogenic portions as described above. For example, the vaccine may comprise one or more hotspots from an ORF in combination with one or more hotspots from a different ORF, etc. Each immunogenic portion may be presented separately in the vaccine composition or may be linked in a single construct. In one embodiment there are at least two discrete immunogenic portions in the vaccine, more preferably there are at least three, four, five, six, seven, eight, nine, ten, fifteen, twenty, twenty five or thirty separate immunogenic portions in the vaccine. It is most preferable that the vaccine will comprise a combination of hotspot regions identified in FIGS. 16 and 17.

The immunogenic portions may be presented in the vaccine composition as amino acid portions (peptides) or may be composed of polynucleotides eg DNA or RNA (eg mRNA).

In one embodiment, the vaccine composition comprises one or more epitopes or hotspot regions identified herein (preferably those identified in any of FIGS. 13-18, preferably 15 or 16, more preferably 17 or 18) within orf1ab. The vaccine composition may further comprise one or more epitopes or hotspot regions identified herein within any of S, orf3a, E, M, orf6, orf8 or N.

In one embodiment, the vaccine composition comprises one or more epitopes or hotspot regions identified herein (preferably those identified in any of FIGS. 13-18, preferably 15 or 16, more preferably 17 or 18) within orf3a. The vaccine composition may further comprise one or more epitopes or hotspot regions identified herein within any of S, orf1ab, orf6, orf8, E, M or N.

In one embodiment, the vaccine composition comprises one or more epitopes or hotspot regions identified herein (preferably those identified in any of FIGS. 13-18, preferably 15 or 16, more preferably 17 or 18) within orf6. The vaccine composition may further comprise one or more epitopes or hotspot regions identified herein within any of S, orf1ab, orf8, orf3a, E, M or N.

In one embodiment, the vaccine composition comprises one or more epitopes or hotspot regions identified herein (preferably those identified in any of FIGS. 13-18, preferably 15 or 16, more preferably 17 or 18) within orf8. The vaccine composition may further comprise one or more epitopes or hotspot regions identified herein within any of S, orf1ab, orf3a, orf6, E, M or N.

In one embodiment, the vaccine composition comprises one or more epitopes or hotspot regions identified herein (preferably those identified in any of FIGS. 13-18, preferably 15 or 16, more preferably 17 or 18) within S. The vaccine composition may further comprise one or more epitopes or hotspot regions identified herein within any of orf1ab, orf3a, orf6, orf8, E, M or N.

In one embodiment, the vaccine composition comprises one or more epitopes or hotspot regions identified herein (preferably those identified in any of FIGS. 13-18, preferably 15 or 16, more preferably 17 or 18) within M. The vaccine composition may further comprise one or more epitopes or hotspot regions identified herein within any of orf1ab, orf3a, orf6, orf8, S, E, or N.

In one embodiment, the vaccine composition comprises one or more epitopes or hotspot regions identified herein (preferably those identified in any of FIGS. 13-18, preferably 15 or 16, more preferably 17 or 18) within E. The vaccine composition may further comprise one or more epitopes or hotspot regions identified herein within any of orf1ab, orf3a, orf6, orf8, S, M or N.

In one embodiment, the vaccine composition comprises one or more epitopes or hotspot regions identified herein (preferably those identified in any of FIGS. 13-18, preferably 15 or 16, more preferably 17 or 18) within N. The vaccine composition may further comprise one or more epitopes or hotspot regions identified herein within any of orf1ab, orf3a, orf6, orf8, S, E or M.

The coronavirus vaccine composition of the present invention may comprise one or more epitopes found within the following table:

TABLE 1
List of further preferred epitope sequences
found within the proteome of SARS-CoV-2.

| SEQ ID | | Protein/ | First | Last |
|---|---|---|---|---|
| NO: | Sequence | ORF | AA No | AA No |
| 1 | CTDDNALAY | orf1ab | 4163 | 4171 |
| 2 | NLIDSYFVV | orf1ab | 4456 | 4464 |
| 3 | TMADLVYAL | orf1ab | 4515 | 4523 |
| 4 | IPRRNVATL | orf1ab | 5916 | 5924 |
| 5 | RLFRKSNLK | S | 454 | 462 |
| 6 | KIADYNYKL | S | 417 | 425 |
| 7 | NYNYLYRLF | S | 448 | 456 |
| 8 | LLFNKVTLA | S | 821 | 829 |
| 9 | FTSDYYQLY | ORF3a | 207 | 215 |
| 10 | HVTFFIYNK | ORF3a | 227 | 235 |
| 11 | YFTSDYYQL | ORF3a | 206 | 214 |
| 12 | YYQLYSTQL | ORF3a | 211 | 219 |
| 13 | SVLLFLAFV | E | 16 | 24 |
| 14 | SEETGTLIV | E | 6 | 14 |
| 15 | LIVNSVLLF | E | 12 | 20 |
| 16 | FVSEETGTL | E | 4 | 12 |
| 17 | SELVIGAVI | M | 136 | 144 |
| 18 | TSRTLSYYK | M | 172 | 180 |
| 19 | ATSRTLSYY | M | 171 | 179 |
| 20 | LSKSLTENK | ORF6 | 40 | 48 |
| 21 | NLIIKNLSK | ORF6 | 34 | 42 |
| 22 | YIDIGNYTV | ORF8 | 73 | 81 |
| 23 | FLEYHDVRV | ORF8 | 108 | 116 |
| 24 | FTINCQEPK | ORF8 | 86 | 94 |
| 25 | NYTVSCLPF | ORF8 | 78 | 86 |
| 26 | EYHDVRVVL | ORF8 | 110 | 118 |
| 27 | LLLDRLNQL | N | 222 | 230 |
| 28 | KPRQKRTAT | N | 257 | 265 |
| 29 | KSAAEASKK | N | 249 | 257 |
| 30 | QRNAPRITF | N | 9 | 17 |

The above epitope sequences are also considered able to stimulate a broad adaptive immune response across a plurality of HLA types, for either MHC Class I and/or MHC Class II immunogenicity.

In some embodiments of the invention, the vaccine composition may comprise one or more epitopes found within Table 1. In other embodiments of the invention, the vaccine composition may comprise one or more epitopes found within Table 1, and also one or more epitopes found within any of the hotspot regions identified in FIGS. 1-10 and/or 13-18.

In some embodiments, the vaccine composition may comprise one or more epitopes according to the present invention that are considered able to stimulate a broad adaptive immune response across a plurality of HLA types for MHC

Class I. In other embodiments, the vaccine composition may comprise one or more epitopes according to the present invention that are considered able to stimulate a broad adaptive immune response across a plurality of HLA types for MHC Class II. In a preferred embodiment, the vaccine composition may comprise one or more epitopes that are considered able to stimulate a broad adaptive immune response across a plurality of HLA types for both MHC Class I and MHC Class II.

It is envisaged that the coronavirus vaccine composition of the present invention may further comprise tertiary protein structures, or domains thereof, of SARS-CoV-2 proteins, such as S protein, M protein, E protein, and/or N protein. In some embodiments, the composition of the present invention may further comprise full recombinant SARS-CoV-2 spike(S) protein, or one or more domains thereof.

The skilled person would appreciate that the one or more epitopes of the present invention, as well as any other protein or domain embodiments, or candidate regions/immunogenic portions/hotspots embodiments, may be comprised within, or encoded by, a cassette. Furthermore, the vaccine composition may comprise one or more polynucleotides encoding the one or more epitopes, hotspots or immunogenic portions according to the present invention, optionally further comprising any other embodiment therein, such as polynucleotides encoding an S protein, or one or more domains thereof. Said polynucleotides may also be comprised within a cassette.

The vaccine composition of the present invention may be formulated according to conventional techniques, eg as a sub-unit peptide vaccine. As will be appreciated by the skilled person, the vaccine may be formulated as a nucleoside-modified mRNA vaccine, preferably wherein the mRNA is encapsulated in lipid nanoparticles. The mRNA may be modified, for example to replace uridine residues with 1-methyl-3′ pseudouridylyl. Other modification to prevent endo and exo-nuclease degradation will be evident to the skilled person.

The vaccine may also be prepared using conventional vector carrier technologies. For example, presenting the one or more epitopes, hotspot regions or immunogenic portions on one or more replication-deficient adenovirus vectors, vesicular stomatis virus vectors, influenza virus vectors or measles virus vectors.

In some embodiments of the present invention, the vaccine composition may further comprise minor amounts of auxiliary substances such as wetting or emulsifying agents, pH buffering agents, and/or adjuvants which enhance the effectiveness of the vaccine.

The phrase “pharmaceutically acceptable” refers to molecular entities and compositions that do not produce an adverse, allergic or other untoward reaction when administered to a human, as appropriate. The preparation of a pharmaceutical composition that contains the vaccine composition of the present invention will be known to those of skill in the art in light of the present disclosure. Moreover, for human administration, it will be understood that preparations should meet sterility, pyrogenicity, general safety and purity standards. A specific example of a pharmacologically acceptable carrier as described herein is borate buffer or sterile saline solution (0.9% NaCl).

As used herein, “pharmaceutically acceptable carrier” includes any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329).

Examples of adjuvants which may be effective include but are not limited to: granulocyte-macrophage colony-stimulating factor (GM-CSF), aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. Further examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAB-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Thus in some embodiments of the invention the composition may further comprise a pharmaceutically acceptable carrier, diluent, excipient and/or adjuvant. In a preferred embodiment, the composition may further comprise an adjuvant.

In a further aspect of the invention, there is provided a coronavirus vaccine composition according to the first, second or third aspects of the invention, for use in the therapeutic or prophylactic treatment of a coronavirus infection in a subject.

In a further aspect, there is a method for the treatment or prevention of a coronavirus infection, comprising administering to a subject a vaccine composition as defined herein.

In some embodiments, the coronavirus vaccine composition may be used in the therapeutic or prophylactic treatment of any coronavirus infection in a subject. In a preferred embodiment, the coronavirus infection may be caused by SARS-CoV-2, SARS-CoV, or MERS-CoV. In a most preferred embodiment, the coronavirus infection may be caused by SARS-CoV-2.

The one or more compositions of the present invention may be administered to the subject via the parenteral, oral, sublingual, nasal, naso-oral, or pulmonary route. In a preferred embodiment, the one or more compositions is administered via a parenteral route selected from subcutaneous, intradermal, intramuscular, subdermal, intraperitoneal, or intravenous injection. In a most preferred embodiment, administration by the parenteral route may comprise intradermal injection of said one or more compositions. The term “injection” as used herein is intended, for the sake of ease, to encompass any such parental, oral, sublingual, nasal, naso-oral, or pulmonary route.

It is envisaged that administration of the coronavirus vaccine composition according to the present invention would be carried out following an appropriate immunisation regimen. The term “appropriate immunisation regimen” is to be construed as a schedule or timescale of one or more administrations of the compositions of the present invention, which may resultantly yield the most effective results in consideration of immunisation efficacy and safety of the subject to which the composition is being administered. For example, for the therapeutic or prophylactic treatment of COVID-19, an immunisation regimen should be chosen that yields as effective immunisation against SARS-CoV-2 as possible, whilst still maintaining suitable safety for the subject.

In some embodiments of the present invention, the immunisation regimen may comprise a single administration. In other embodiments, the immunisation regimen may comprise multiple administrations, either concomitantly or over an appropriate period of time. In a preferred embodiment, the immunisation regimen may comprise multiple administrations over a period of 14 days.

It is envisaged that the appropriate dosage regimen may be repeated for each subject at a suitable time. In a preferred embodiment, the immunisation regimen may be repeated after one month.

There exists further the possibility to further administer boost immunisations after a more extended period of time. This may be selected as an appropriate measure if a subject's immunoglobulin G (IgG) antibody levels or T-cell response fall below determined protective levels. Thus in some embodiments, an appropriate dosage regimen may be given as a “boost immunisation” after 6 months.

In some embodiments of the present invention, the coronavirus vaccine composition may be administered for the treatment or prevention of infections caused by a virus in combination with one or more other antiviral therapies or other appropriate therapies such as stem cell therapies. Such antiviral therapies may include administration of oseltamivir phosphate (Tamiflu®), zanamivir (Relenza®), peramivir (Rapivab®), baloxavir marboxil (Xofluza®), or lopinavir/ritonavir (Aluvia®). Such antiviral therapies may be administered simultaneously, separately or sequentially with the composition of the present invention. In a further embodiment, the antiviral therapy is administered via the same or different route of administration as the composition of the present invention, for example via intradermal injection.

In another aspect of the invention, there is provided a use of a coronavirus vaccine composition according to the first aspect of the invention, in the manufacture of a medicament for the therapeutic or prophylactic treatment of a coronavirus infection.

The manufacture of said medicament may involve the selecting of one or more epitope sequences or candidate regions/immunogenic portions or hotspots for inclusion in a vaccine from a set of predicted immunogenic candidate amino acid sequences by a method according to any of the preceding aspects of the invention, and synthesising the one or more amino acid sequences or encoding the one or more amino acid sequences into a corresponding DNA or RNA sequence. Said DNA and/or RNA sequences may be inserted into a genome of a bacterial or viral delivery system to create a vaccine, or used naked, or in some other formulation such as lipid nanoparticles to create a vaccine

In a further aspect of the invention, there is provided a diagnostic assay to determine whether a patient has or has had prior infection with SARS-CoV-2 (and for example has developed a protective immune response), wherein the diagnostic assay is carried out on a biological sample obtained from a subject, and wherein the diagnostic assay comprises the utilisation or identification within the biological sample of one or more epitopes according to any of claims 1-15. The term utilisation as used herein is intended to mean that the epitopes of the present invention are used in an assay to identify an (e.g. protective) immune response in a patient. In this context, the epitopes are not the target of the assay, but a component of said assay.

Suitable diagnostic assays would be appreciated by the skilled person, but may include enzyme-linked immune absorbent spot (ELISPOT) assays, enzyme-linked immunosorbent assays (ELISA), cytokine capture assays, intracellular staining assays, tetramer staining assays, or limiting dilution culture assays.

In another embodiment, the in vitro diagnostic test may comprise an immune system component based assay to identify an immune system component within the biological sample that recognises one or more epitopes of the present invention. In this way, the diagnostic assay may utilise the at least one identified candidate region and/or at least one predicted epitope of the present invention.

Typically the diagnostic assay will contain the (e.g. synthesised) at least one identified candidate region and/or predicted epitope of the present invention. In a preferred embodiment, the immune system component may be a T-cell. In another preferred embodiment, the immune system component may be a B-cell.

As an example of such a diagnostic use, a sample, preferably a blood sample, isolated from a patient may be analysed for the presence of T-cells that recognise and bind to epitopes within the candidate regions, or hotspots, contained within the assay that have been identified as part of the present invention. The epitopes identified as part of the present invention are predicted to be presented by HLA molecules, and as such are capable of being recognised by T-cells. Thus, the coronavirus vaccine composition according to the present invention may be used to create a quick diagnostic test or assay. The epitopes identified as part of the vaccine compositions may be further analysed in laboratory testing in order to create such a diagnostic test or assay, thereby significantly reducing the time taken to develop the test compared to traditional laboratory methods.

Such a T-cell diagnostic response would indicate to the skilled person whether the patient has been exposed to an infection by SARS-CoV-2 and has developed a protective immune response, wherein said infection resulted in an observable level of cellular immunity and/or immunological memory.

### Example 1

The first part of the data processing to identify potential epitopes involved the generation of epitope scores for each amino acid position in all the proteins in the SARS-CoV-2 proteome, for 100 HLA types.

For HLA types of MHC class I, the scores assigned to each amino-acid were in the range of 0 to 1, with 1 being the best epitope score. For HLA types of MHC class II, the scores assigned to each amino-acid were in the range of 0 to 100 (percentile ranks), with 0 being the best epitope score. A score for a designated amino-acid was determined as the best score that a peptide overlapping that amino-acid carries in the predictions. All peptides of size 8-12 for class I, and size 15 for class II had been processed by the antigen presentation framework. At this point, one dataset per protein was generated. Each row in the dataset represented the amino-acid epitope scores predicted for one HLA type.

### Example 2

To ascertain whether the regions in a given protein that were most enriched with high epitope scores, in respect to a given set of HLA types, were enriched more than could reasonably be expected by chance, a hypothesis testing framework was implemented.

The raw input datasets were first transformed into binary tracks. For each class I HLA dataset, the epitope scores were transformed to binary (0 and 1) values, such that amino acid positions with predicted epitope scores larger than 0.7 were assigned the value 1 (positively predicted epitope), and the rest were assigned the value 0. Similarly, for class II HLA datasets, amino acid positions with predicted epitope scores 10 or smaller were assigned the value 1, otherwise 0. These cut-off thresholds were relatively conservative. Each binary track could effectively be presented as a list of intervals of consecutive ones segments, with consecutive zeros in between, forming inter-segments or gaps.

### Example 3

For a group of k HLA binary tracks, a test statistic Si was calculated for each hotspot bᵢ of given size m, dividing the protein in n hotspots (e.g. m=100 amino acids for the larger proteins). For a single HLA track, a test statistic si was calculated:

$${s}_{i} = {\sum }_{j = 1}^{m} {b}_{i , j} * {weight}_{k} \qquad ( 1 )$$

Wherein the weight is default 1.0, however can also represent frequency of the HLA track in the population under analysis.

Then:

$${S}_{i} = \frac{{\sum }_{i = 1}^{k} {s}_{i}}{k} \qquad ( 2 )$$

Is the average number of amino acids predicted to be epitopes (epitope enrichment) of the hotspot bᵢ, across the selected HLA types.

### Example 4

A Monte Carlo-based simulation was carried out to estimate the statistical significance of each observed hotspot.

A null model was defined, as the generative model of the HLA tracks, if they were generated by chance. From the null model, through sampling, the null distribution of the test statistic Si arose. To sample from the null model, each of the k HLA tracks was divided into segments and gaps, which were then shuffled to produce a randomised HLA track. This was repeated 10,000 times, to produce 10,000 samples of Si statistic for each hotspot. For each hotspot, the p-value was estimated as the proportion of the samples that were equal or larger than the truly observed enrichment. Further, the generated p-values were adjusted for multiple testing with the Benjamin-Hochberg procedure to control for a false discovery rate (FDR) of 0.05. A Benjamini-Yekutieli procedure could also be used as an alternative.

All hotspots that resulted in an adjusted p-value of lower than 0.05 were considered to be statistically significant hotspots across the selected HLA group.

### Example 5

The following example describes a “digital twin” approach for peptide or hotspot selection process: a method and system for selecting a small set of candidate peptides or hotspots for inclusion in a vaccine such that the likelihood that every member of a population has a positive response to the vaccine is maximised.

In the “digital twin” framework, synthetic populations were simulated, and an optimal selection of peptides or hotspots was made with respect to that simulation. The final peptide selection may then be based on commonly selected peptides in all simulations.

A population was considered as a set C of “digital twin” citizens c, and a vaccine as a set V of vaccine elements v. We model the likelihood that each citizen has a positive response to a vaccine, P(R=+|C,V), as follows:

$$P ( R = + \vert C , V ) = {min}_{c ∈ C} P ( R = + \vert c , V )$$

Our goal was then to select the vaccine V that maximizes this likelihood.

$${max}_{V} P ( R = + \vert Pop , V ) = {max}_{V} {min}_{c ∈ C} \{ P ( R = + \vert c , V ) \}$$

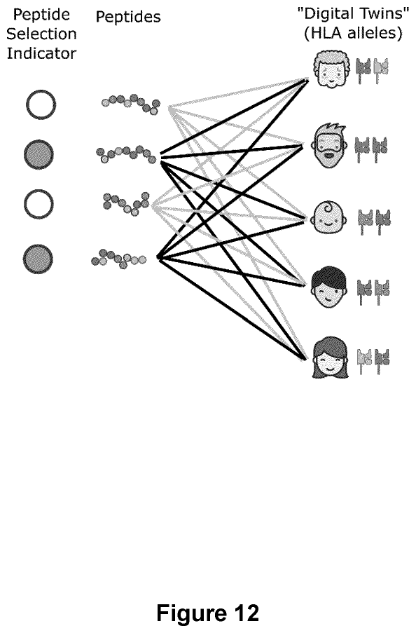

This maximin problem was approached as a type of weighted bipartite graph matching problem. FIG. 12 gives an overview of the problem setting.

We performed a set of Monte Carlo simulations to assign a score to each vaccine element. In each simulation, we performed the following steps.
(1) Select a set of candidate vaccine elements for inclusion in the vaccine.

The vaccine elements could also be the “hotspots” or anything else.
(2) Create a set of “digital twins” of members of a population.

In the context of the present invention, a digital twin was a set of HLA alleles. We had downloaded full HLA genotypes from actual citizens from a set of high-quality samples from the Allele Frequency Net Database (AFND). Thus, we could ensure our digital twins have HLA backgrounds that were accurate.

AFND assigned each sample to a region based on where the sample came from (e.g., “Europe” or “Sub-Saharan Africa”). In an offline step, we create a posterior distribution over genotypes based on the observations in each sample and an uninformative (Jeffreys) prior distribution. Creating a population thus consisted of the following steps:
(i) Specify a (Dirichlet) prior distribution over regions and a population size;
(ii) Sample a multinomial distribution over regions based on the Dirichlet prior;
(iii) Sample population counts from all regions based on the multinomial distribution;
(iv) Sample genotypes from the posterior Dirichlet over genotypes for each region.

The digital twin concept could also include sampling the strain, mutations, etc., for the virus in that patient. These sampling distributions can also be posterior distributions based on prior assumptions and observed data.
(3) Create a graph in which each vaccine element i is connected to each “digital twin” j. The weight of the edge is the log likelihood that the vaccine element will result in a “positive” response in that patient. (We refer to this value as p_{i,j}.)
(4) Assume that the likelihood that a vaccine element will elicit a positive response when it is included in the vaccine is independent of other included elements. In this case, then, the log likelihood that a citizen has a positive response is equal to the sum of the log likelihoods that each individual vaccine element results in a response. We refer to this overall log likelihood of response for a particular citizen as xⱼ^{citizen}.

In terms of the graph, we called the edges from a vaccine element to a citizen as “active” when the vaccine element is selected. Then, the log likelihood of response for a citizen was the sum of all active incoming edges.
(5) Select a set of vaccine elements (of a fixed weight) such that the likelihood that each patient has a positive response is maximized. Since the likelihood that a patient responds was equal to the sum of active edges, this selection could be framed as an integer linear program (ILP) and provably, optimally solved using conventional ILP solvers. We used binary indictor variables xᵢ^{peptide} to indicate selected peptides.

### Example 6

### Peptide Pool Creation and Validation

93 unique peptides were selected for validation in convalescent patient samples. The peptides were sorted into seven allele-specific peptide pools, as well as three pan-allele pools. The peptides included in some of the pan-allele pools overlap with those in the allele-specific pools, but each peptide appears in only one allele-specific pool.

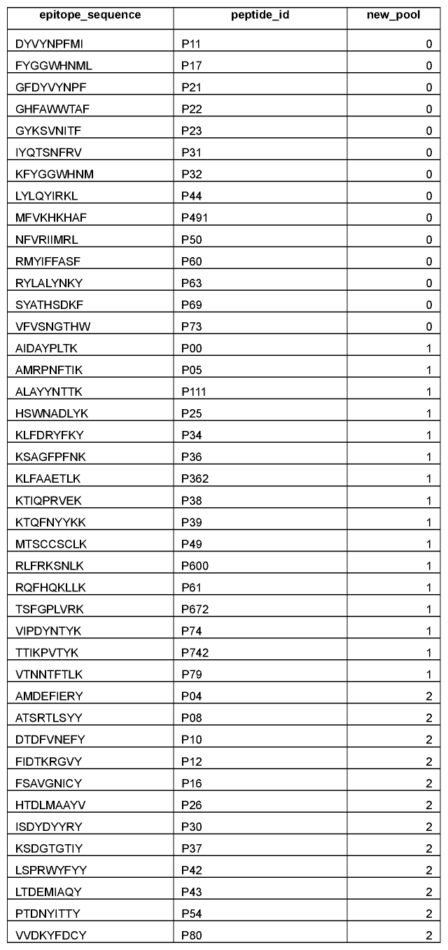

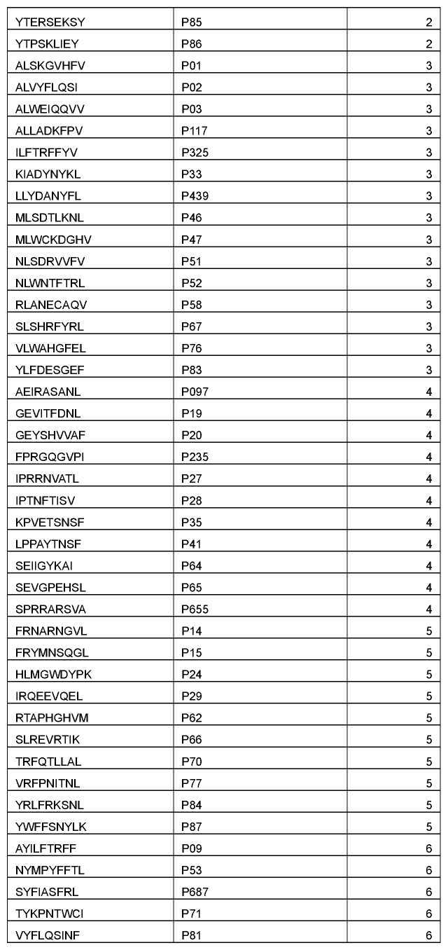

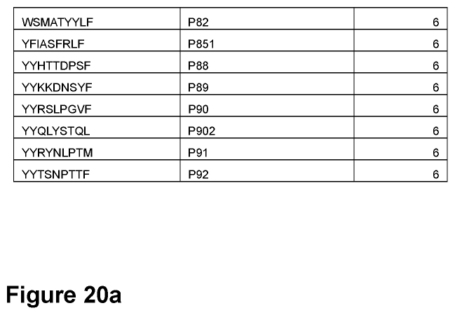

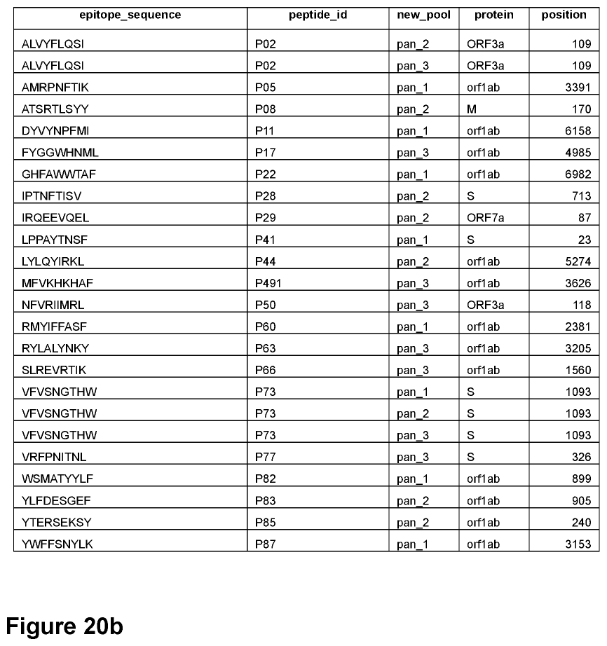

FIG. 20 shows the final allocation of peptides to pools.

Unless otherwise noted, the following HLA class I alleles were considered in this analysis:
A0101
A0201
A0301
A1101
A2301
A2402
B0702
B4001
C0701
C0702

Unless otherwise noted, the following HLA binding prediction methods were used in this analysis:
NetMHCPan
NetMHC
MHCFlurry
A custom ResNet model, included in the “ResERT” package from NEC Laboratories Europe GmbH (NLE)

For HLA presentation prediction, a custom ResNet model trained by NLE was used for making predictions.

The “AP” and “IP” scores are the “antigen presentation” and “immune presentation” scores calculated as disclosed herein.

### General Filtering

For each possible peptide and considered set of alleles, we made predictions with each binding tool and our presentation model. A conservation score was also calculated which accounts for how many sars-cov-2 genomes in which the peptide occurs.

All selection methods used the following filtering approach to identify a set of high-quality candidate peptides for validation.
Binding predictions must be greater than 500 nM (>4.7) for at least three of the four binding methods.
The likelihood of presentation must be >70%.
The peptide must appear in more than 90 (out of 119 collected at that time) genomes.

### Peptide Selection for Allele-Specific Peptide Pools

The selection and pool creation was as follows. Some of the selection steps resulted in duplication peptides (e.g., one peptide predicted to be a strong binder to multiple HLA alleles, likely to appear twice in Step 2 below). Only unique peptides are retained.
1. We filtered for high-quality candidate peptides as described above.
2. We selected peptides which are the top-5 for each allele, sorted by likelihood of presentation (ties broken by mean prediction of all four binding tools).
3. We selected 8 peptides each based on AP and IP scores. (See below 3a.)
4. We selected 8 peptides based on the preferred hotspots identified in FIGS. 17 and 18 and IP scores. (See below 3b.)
5. We selected 4 peptides predicted to be strong binders but with a low likelihood of presentation (see below 3c).
6. We selected the remaining top-7 peptides (again, sorted by presentation then binding) from common alleles (A0101, A2402, A0201, A0301).
7. The 93 unique peptides were sorted into seven pools by minimizing the difference in predicted binding scores for all HLA alleles in each peptide pool. This minimization was performed using a standard greedy hill climbing algorithm.

### 3a AP and IP Peptide Selection

1. We filtered for high-quality candidate peptides as described above.
2. We used either the AP or IP scores to calculate likelihood of immune response.
3. These scores were used in the integer linear programming optimization routine to select peptides for maximizing population coverage across 110 different populations (10 each of global plus population specific).
4. We hand-selected 8 peptides based on optimization results.

### 3b Hotspot Peptide Selection

1. We filtered for high-quality candidate peptides as described above.
2. We further filtered and only included peptides which overlapped one of the preferred AP or IP preferred hotspots (FIGS. 17 and 18).
3. We used the integer linear programming optimization routine to select peptides for maximizing population coverage across 110 different populations (10 each of global plus population specific), using IP scores to calculate likelihood of response.
4. We hand-selected 8 peptides based on optimization results.

### 3c Strong Binder but Low Likelihood of Presentation Peptide Selection

We selected four peptides (one each for the alleles A0101, A0201, A0301, and A2402) that were predicted to be strong binders but that had a low predicted likelihood of presentation were selected.
1. We filtered for high-quality candidate peptides as described above.
2. All candidate peptides with a predicted likelihood of presentation 50% or greater were removed. (Thus, “weak” binders have been removed in Step 1, and “high likelihood” of presentation have been removed in this step.)
3. Of the remaining peptides, those with the highest average predicting binding score for the A0101, A0201, A0301, and A2402 were retained.

### 4. Results

### 1. Allele-Specific Pool Results

Pools 0-6 were tested using fresh blood samples collected from patients. 7 patients (presenting fever but not hospitalized; confirmed positive for COVID with PCR; samples taken after recovery) were tested; 3 controls were also tested. Experimental positive and negative experimental controls were also included. No HLA typing was available.

ELISpot assays were used to test for IFNg response.

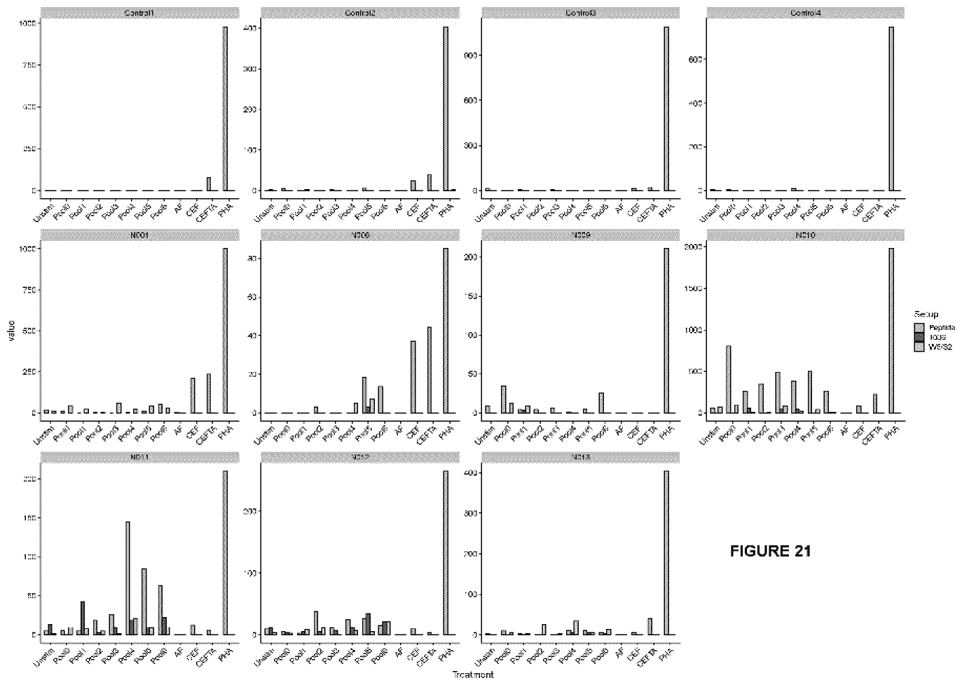

FIG. 21 shows the results, in terms of spots per 3×10⁵ cells. In addition to the pools, the following controls are included (as indicated in the plots):
Unstimulated
AF: autofluorescence. i.e., spots resulting from artefacts such as antibody precipitates.
Cytomegalovirus/Epstein-Barr virus/influenza (CEF)
Cytomegalovirus, Epstein Barr virus, Influenza virus, Tetanus toxin, and Adenovirus 5 (CEFTA)
Phytohemagglutinin (PHA)
Tu39—Anti HLA Class II (DR, DP, and most DQ) antibody
w6/32—Anti-HLA Class I antibody

### 2. Pan-Allele Pool Results

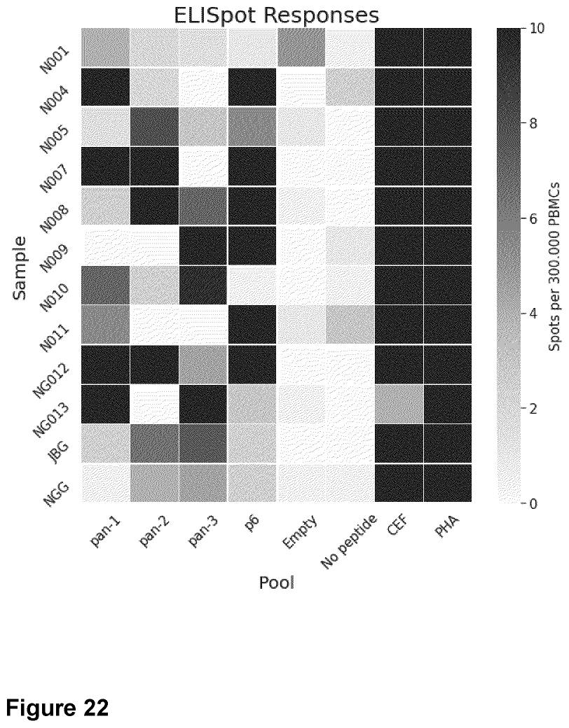

The pan-allele pools were tested using fresh blood samples collected from patients. 10 patients (presenting fever but not hospitalized; confirmed positive for COVID with PCR; samples taken after recovery) were tested (N001, N004 etc); 2 controls were also tested (the “JBG” and “NGG” rows in the results heatmap FIG. 22). Experimental positive and negative experimental controls were also included. No HLA typing was available.

ELISpot assays were used to test for IFNg response.

FIG. 22 shows the results, in terms of spots per 300,000 cells. In addition to the pools, the following controls are included (as indicated in the plot).
Empty—nothing is included in the well at all.
No peptide—the sample but not peptide was included. This matches the “Unstimulated” setting in the allele-specific pool results.
CEF
PHA

### 5. Conclusions

The results demonstrate that at least one of the pan-allele pools resulted in a positive immune response, above what was observed in the negative controls (FIG. 22). Further, five of the seven patients responded to at least one of the allele-specific pools (FIG. 21), while all of the allele-specific pools led to an immune response in at least one patient. None of the pools resulted in a significant response in the negative control settings.

Thus, these results show that these peptides are associated with immune responses in recovered patients, and they do not result in responses from patients who have not tested positive for COVID.

### Example 7

### Objective

The aim of the study was to generate proof-of-concept data that demonstrates that the hotspot regions identified in silico using the NEC Immune Profiler and subsequent Monte Carlo simulation analysis are immunogenic i.e., minimal epitopes contained within the hotspots are recognized by T-cells from convalescent donors who have recovered from SARS-CoV-2 infection.

### Method

### 1. Identification of the Minimal Epitopes.

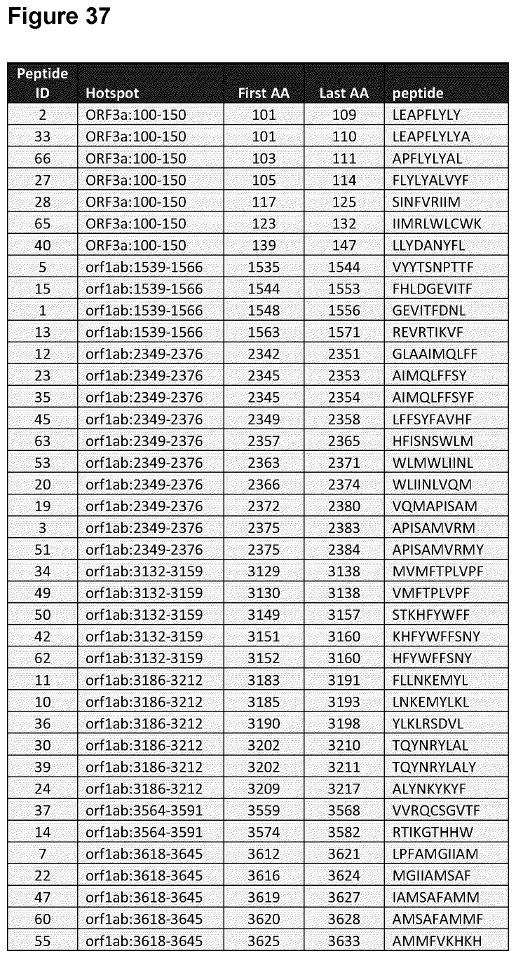

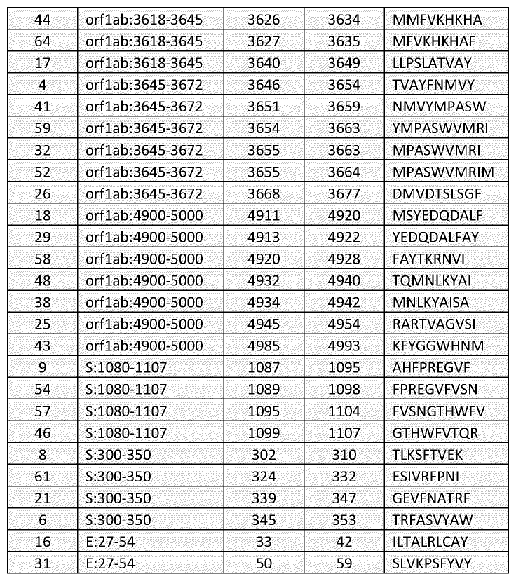

For each hotspot identified in Table 2 (below) every possible 9mer and 10mer peptide permutation was created in silico by tiling across the peptide sequence and flanking regions. Predicted cell surface presentation scores (AP scores) and immunogenicity scores (IP scores) were then generated for the most common HLA-A and HLA-B alleles in the Norwegian population; HLA-A*01:01, HLA-A*02:01, HLA-A*03:01, HLA-A*23:01, HLA-A*29:02, HLA-B*07:02, HLA-B*08:01, HLA-B*15:01, HLA-B*15:02 HLA-B*40:01 & HLA-B*44:02. Peptides with AP & IP scores above 0.7 and 0.5 respectively were synthesized for subsequent immunogenicity testing. 65 (mutually exclusive) peptides from the 12 hotspots were successfully synthesized and subsequently tested in total (see FIG. 37).

TABLE 2
The test peptides from the selected hotspots

| | # epitopes identified | | | |
|---|---|---|---|---|
| | OL | | OL | |
| Hotspot ID | N-terminus | Exact | C-terminus | Total |
| ORF3a:100-150 | 0 | 7 | 0 | 7 |
| orf1ab:1539-1566 | 1 | 2 | 1 | 4 |
| orf1ab:2349-2376 | 3 | 4 | 3 | 10 |
| orf1ab:3132-3159 | 2 | 1 | 2 | 5 |
| orf1ab:3186-3213 | 2 | 3 | 1 | 6 |
| orf1ab:3564-3591 | 1 | 1 | 0 | 2 |
| orf1ab:3618-3645 | 2 | 5 | 1 | 8 |
| orf1ab:3645-3672 | 0 | 5 | 1 | 6 |
| orf1ab:4900-5000 | 0 | 7 | 0 | 7 |
| S:1080-1107 | 0 | 4 | 0 | 4 |
| S:300-350 | 0 | 3 | 1 | 4 |
| E:27-54 | 0 | 1 | 1 | 2 |
| Total | 11 | 43 | 11 | 65 |

Preferred hotspots from FIGS. 16 & 17 are shown in bold text while other hotspots that were evaluated are non-bolded text.

### 2. Immunogenicity Testing

### SARS-CoV-2 Donors

Blood samples were collected from donors with confirmed SARS-CoV-2 PCR-positive status 3-12 weeks after resolution of disease. All the donors had self-limiting disease associated with mild symptoms and were not hospitalized. Peripheral blood mononuclear cells (PBMCs) were isolated from the blood using centrifugation and then used for subsequent immunogenicity testing to determine the antigen-specific T cell response directed against the selected test SARS-CoV-2 epitopes. Even though the test peptides were selected based on the most common HLA-A and HLA-B alleles in the Norwegian population, the patients in the study were not HLA-typed (at the time of immunogenicity testing) and it is quite likely that many were not ethnic Norwegians as COVID-19 was more predominant in the non-ethnic Norwegian population when the samples were collected.

### T-Cell Profiling

All PBMC samples were tested for proliferative (3H-thymidine incorporation) and cytokine (IFN-γ) responses to the individual 65 selected test peptides.

Quantifying IFN-γ Secretion by T-Cells after Restimulation with Predicted Epitopes

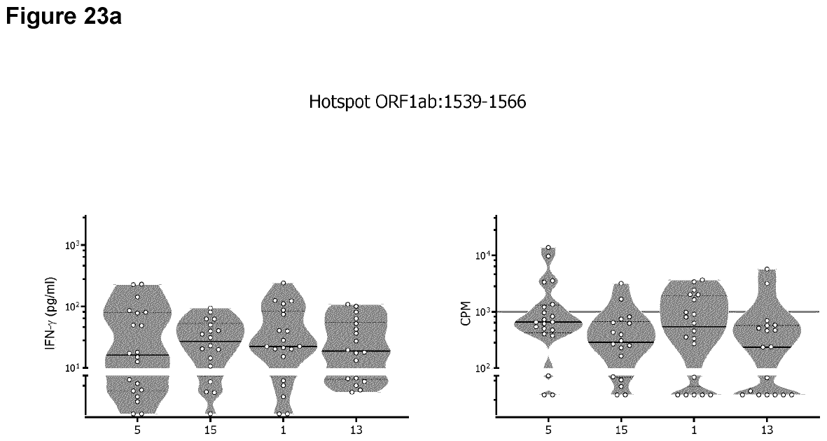

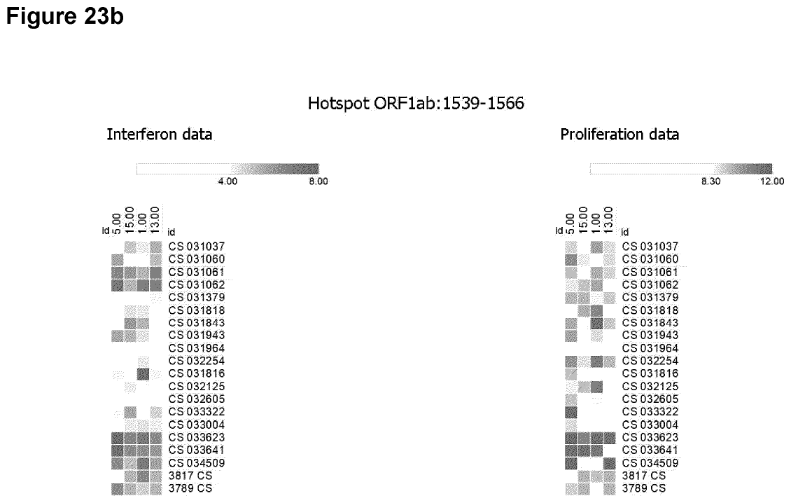

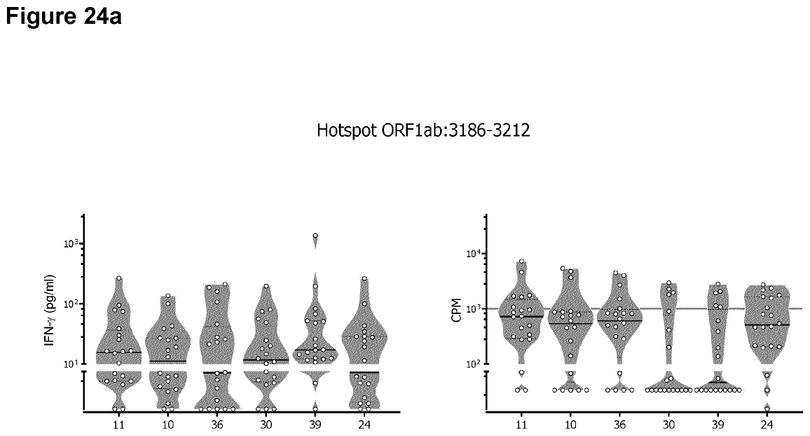

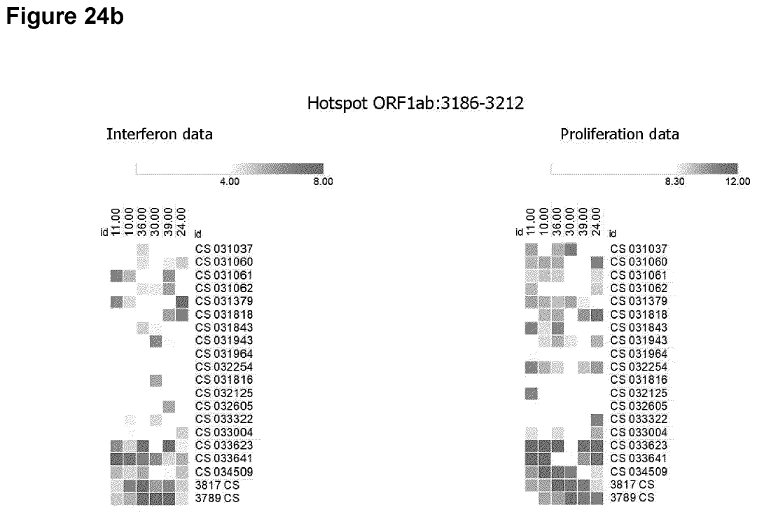

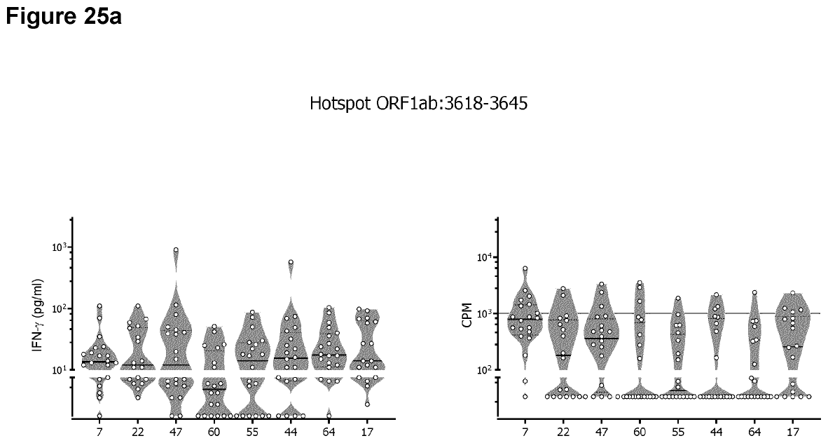

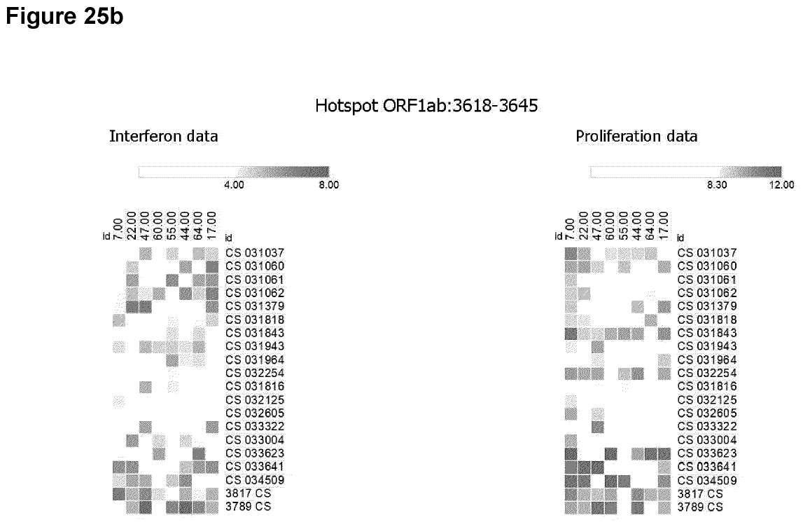

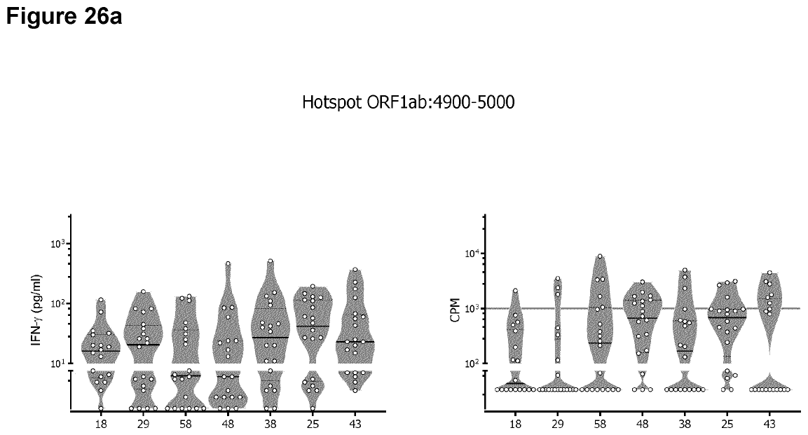

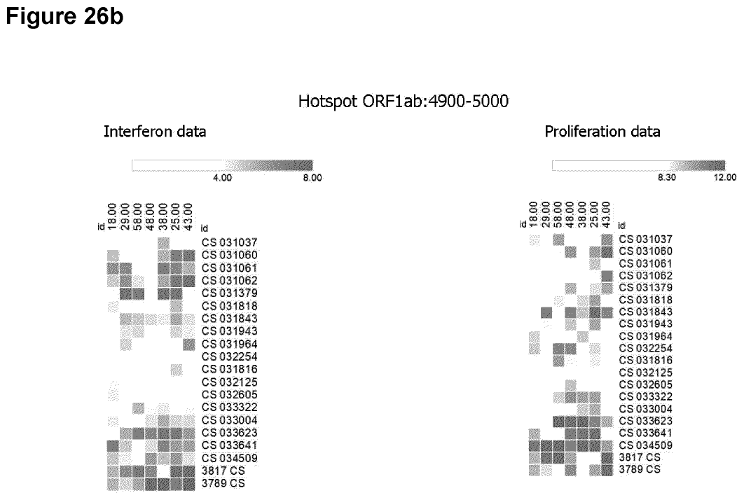

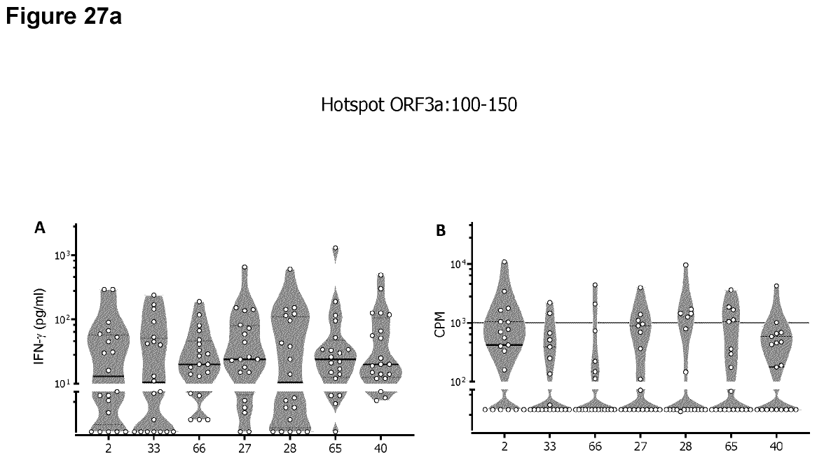

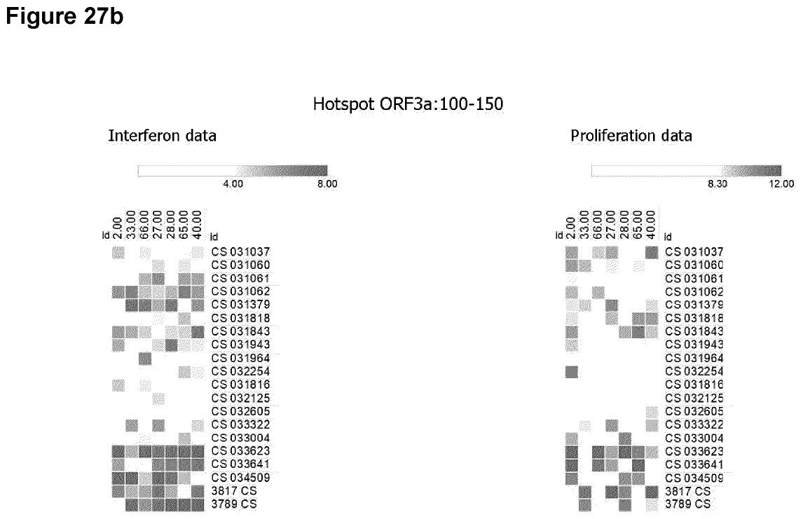

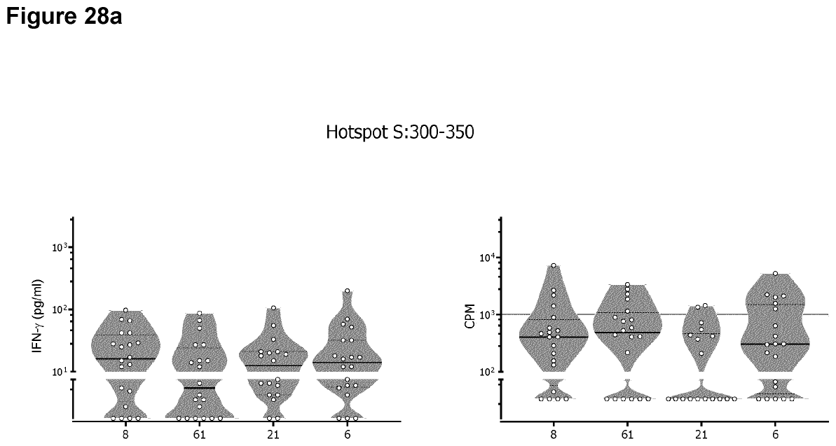

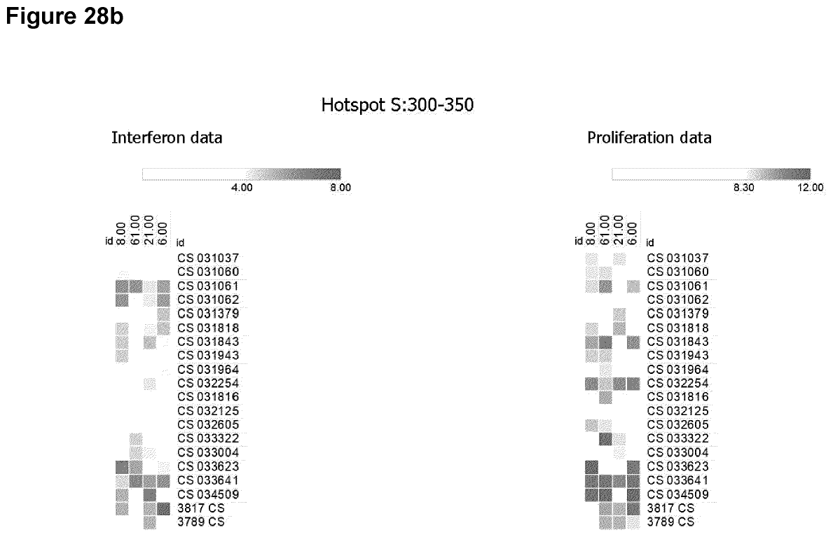

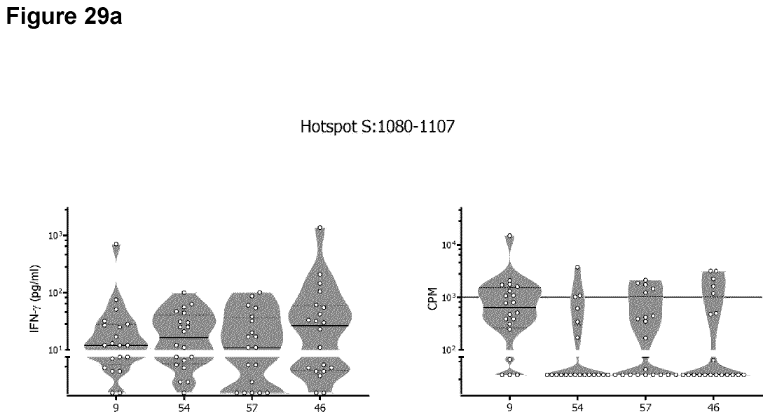

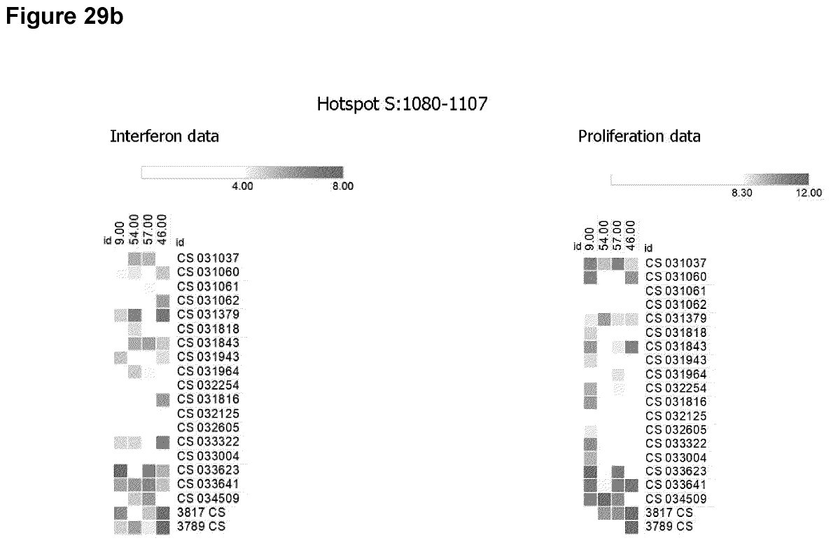

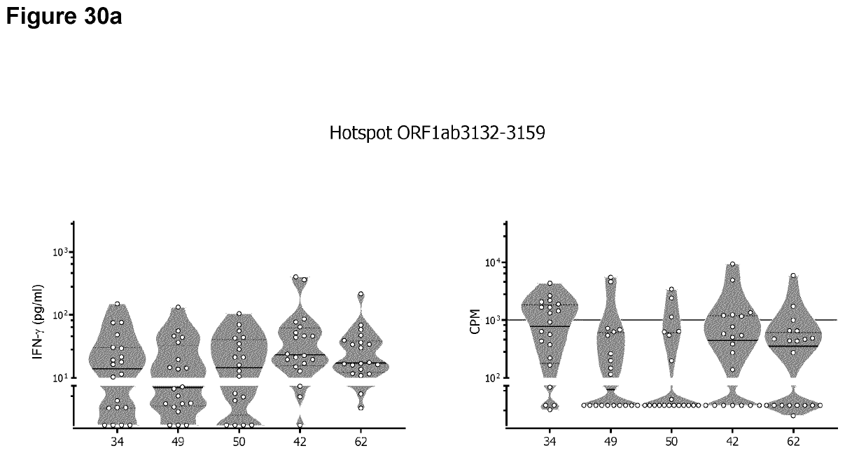

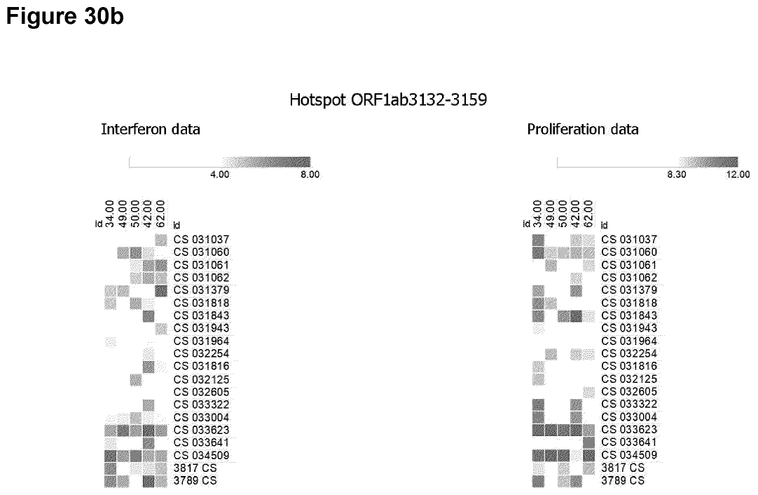

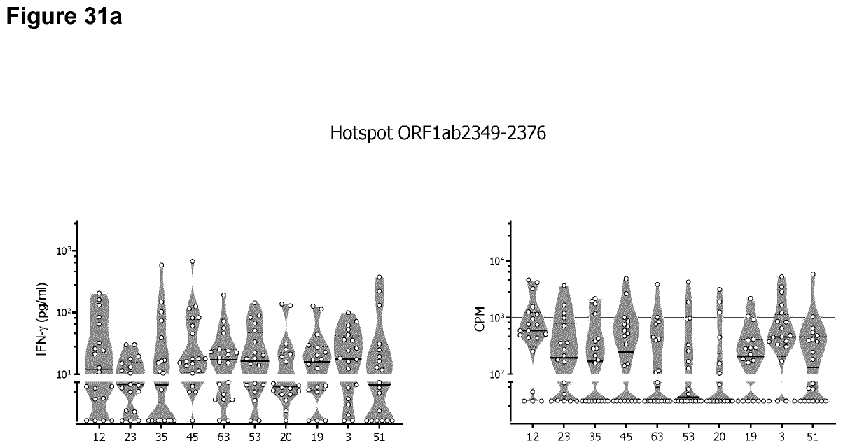

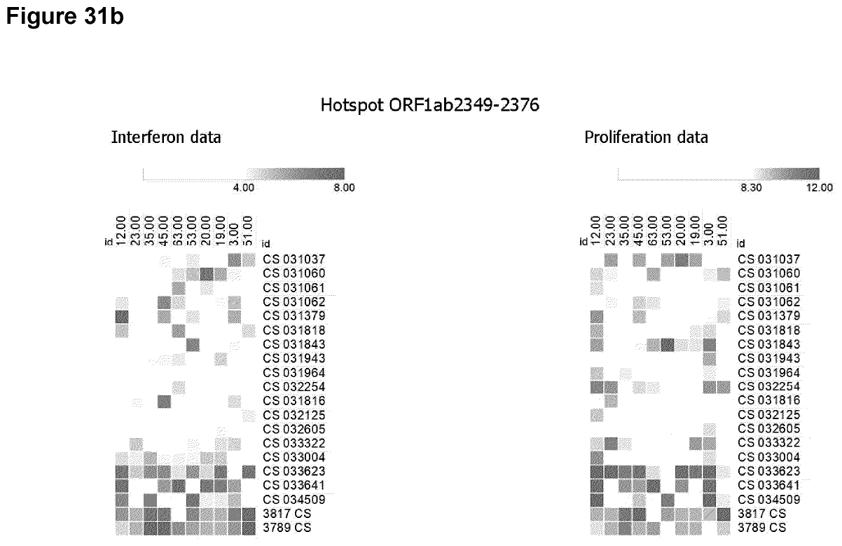

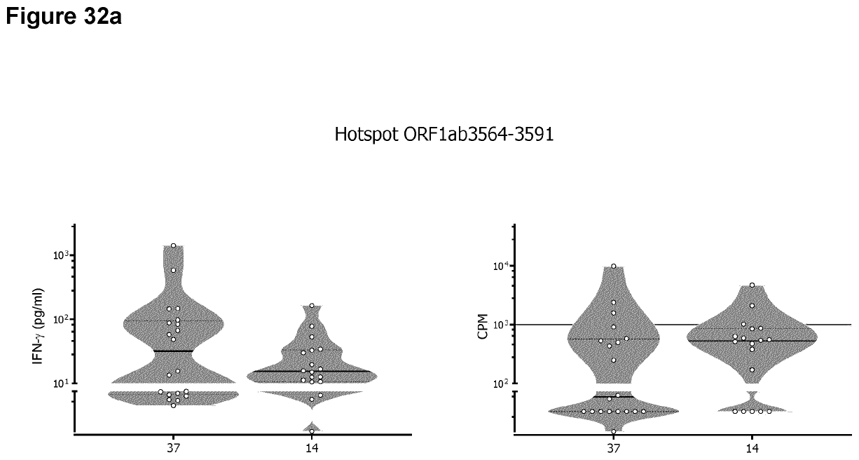

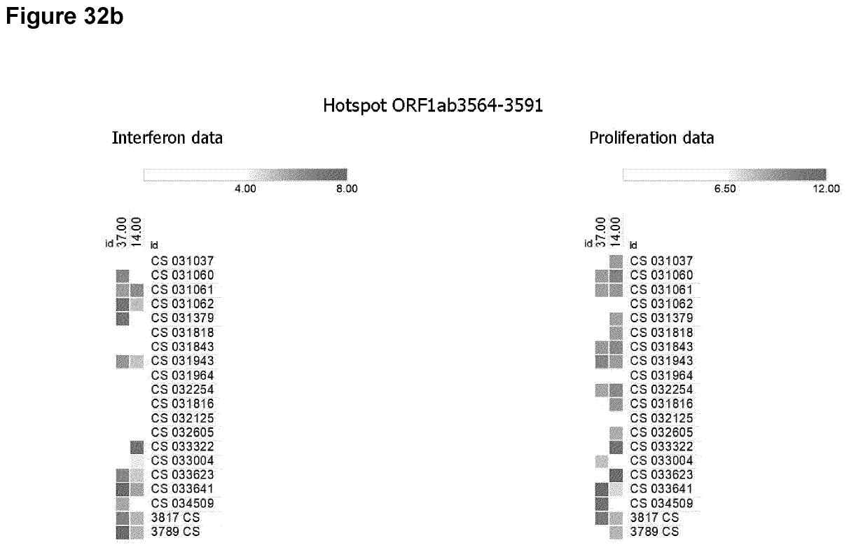

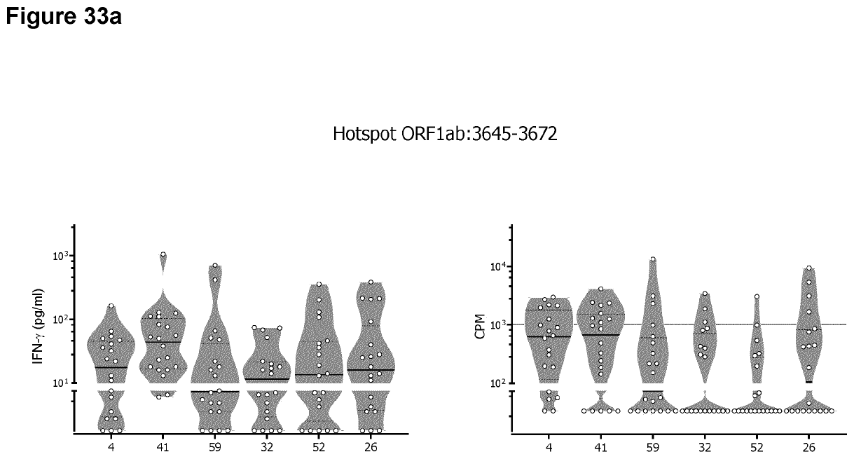

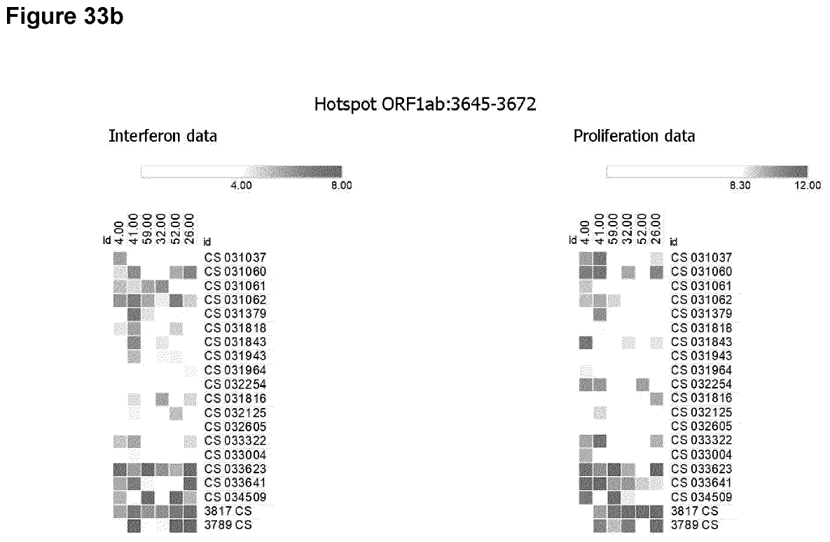

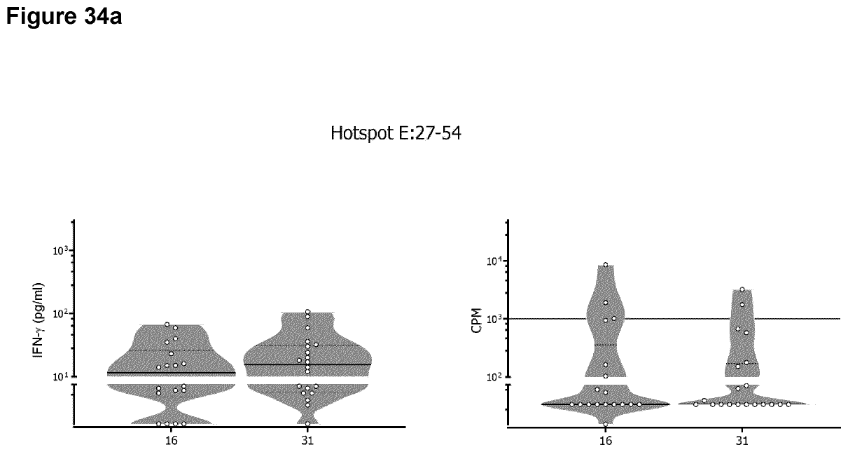

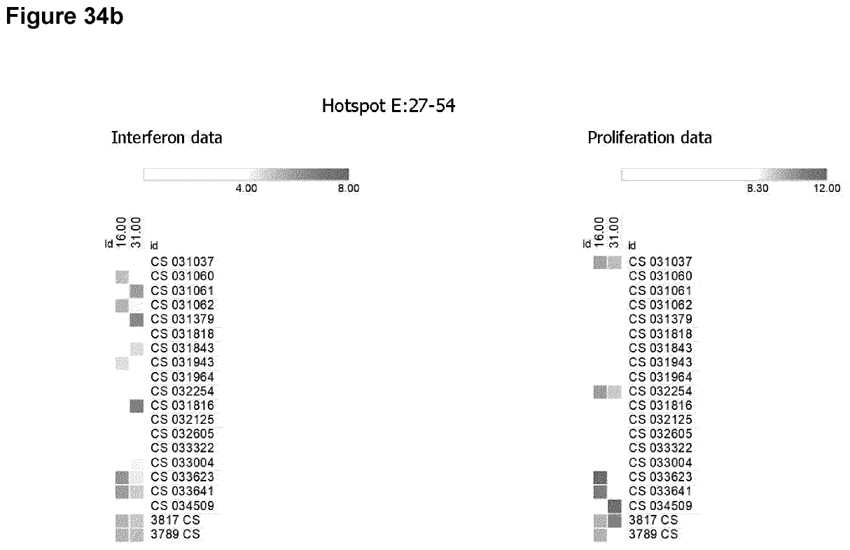

In brief, approximately 5×10⁵ PBMCs (from an individual patient) was added per well to a 96-microtiter plate and restimulated by the addition of 1 ug of test peptide (tested individually). PBMCs were also restimulated with media alone as a negative control or PMA as a maximum stimulation control. After a 3-day incubation, supernatants were removed and frozen for subsequent quantification of IFN-γ by ELISA. A commercial capture ELISA kit was used to quantify the level of secreted IFN-γ, and plates were developed using HRP. The level of IFN-γfor each patient/peptide combination was calculated using a titration curve. The results for each tested patient/peptide combination associated with a specific predicted hotspot was plotted in a violin plot and associated heatmap as shown below in FIGS. 23 to 34.

Measuring T-Cell Proliferation Responses after Restimulation with Predicted Epitopes

The restimulated PBMCs (from the above experiment) were subsequently incubated with 3H-thymidine for a further 3 days before being harvested and the amount of incorporated 3H-thymidine determined using a scintillation beta-counter and measured as counts per minute (CPM). Background CPM values for the negative controls were subtracted from the CPM values measured in the experiment wells restimulated with the individual test peptides. The net CPM results for each tested patient/peptide combination associated with a specific predicted hotspot was plotted in a violin plot and associated heatmap as shown below in FIGS. 23 to 34.

### 3. Results

### Summary Overview of the Results Per Hotspot

The IFN-γ and T cell proliferation response for each hotspot and each individual patient are shown in violin plots and associated heatmaps in FIGS. 23 to 34.

### An Epitope-Centric Overview

100% of the tested epitopes stimulated antigen-specific T-cell responses (were immunogenic) in the PBMCs from at least one donor when using an IFN-γ threshold of 20 pg/ml and a proliferation threshold of 500 CPM. 100% and 83% of the epitopes were immunogenic (in at least one donor) when using an IFN-γ threshold of 100 pg/ml and a proliferation threshold of 1000 CPM respectively (see table 3 below).

TABLE 3

| | # epitopes that stimulated a response | | | |
|---|---|---|---|---|
| | in at least 1 donor | | | |
| | IFN-γ | | Proliferation | |
| | 20 | 100 | 500 | 1000 |
| Hotspot ID | pg/ml | pg/ml | CPM | CPM |
| ORF3a:100-150 | 7/7 | 7/7 | 7/7 | 7/7 |
| orf1ab: 1539-1566 | 4/4 | 3/4 | 4/4 | 4/4 |
| orf1ab:2349-2376 | 10/10 | 8/10 | 10/10 | 10/10 |
| orf1ab:3132-3159 | 5/5 | 5/5 | 5/5 | 5/5 |
| orf1ab:3186-3213 | 6/6 | 6/6 | 6/6 | 6/6 |
| orf1ab:3564-3591 | 2/2 | 2/2 | 2/2 | 2/2 |
| orf1ab:3618-3645 | 8/8 | 5/8 | 8/8 | 8/8 |
| orf1ab:3645-3672 | 6/6 | 5/6 | 6/6 | 6/6 |
| orf1ab:4900-5000 | 7/7 | 7/7 | 7/7 | 7/7 |
| S:1080-1107 | 4/4 | 2/4 | 4/4 | 4/4 |
| S:300-350 | 4/4 | 3/4 | 4/4 | 4/4 |
| E:27-54 | 2/2 | 1/2 | 2/2 | 2/2 |
| Total | 100% | 83% | 100% | 100% |

### A Hotspot-Centric Overview

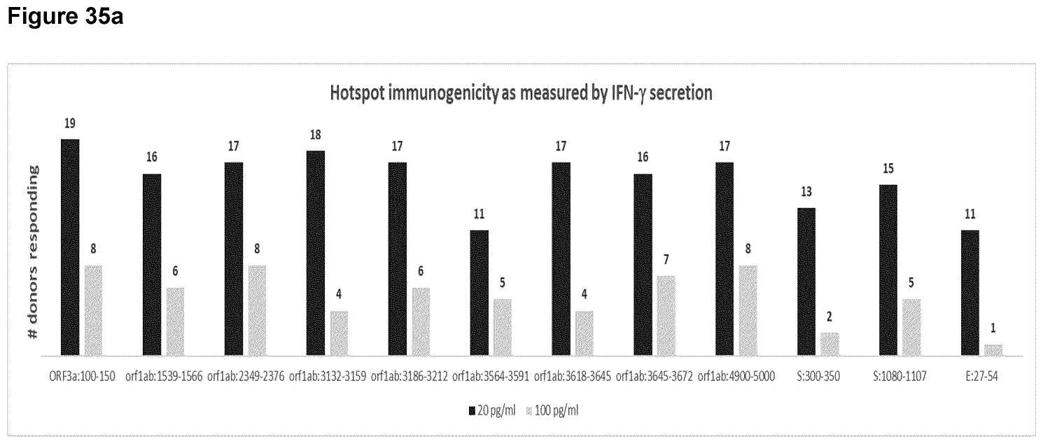

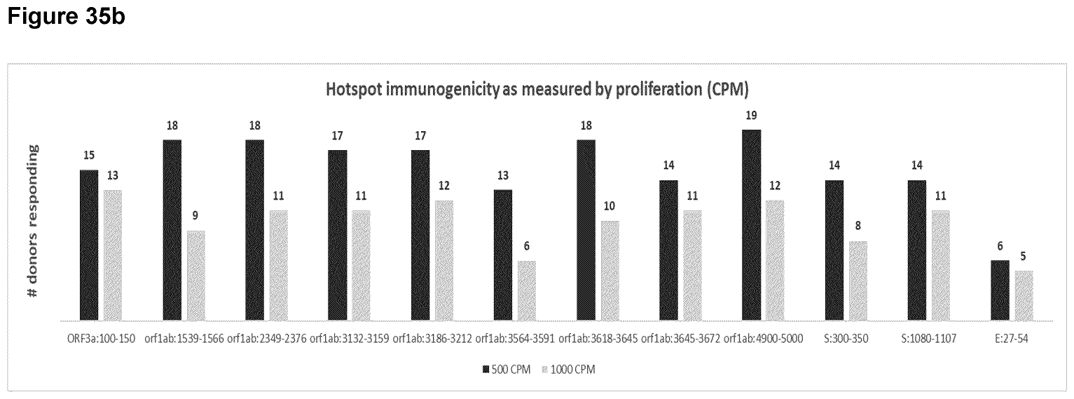

100% of the tested hotspots were shown to be immunogenic in the PBMCs from at least 1 donor using both IFN-γ secretion and T-cell proliferation readouts at both the lower and higher thresholds as shown in FIGS. 35a & 35b.

9/12 and hotspots were immunogenic in 75% of the donors using the lower IFN-γ threshold (20 pg/ml) and 7/12 using the lower proliferation threshold (500 CPM). These percentages were reduced when the higher readout thresholds were applied, although the responses were still highly surprisingly robust, especially when using the proliferation readout.

### A Donor-Centric Overview

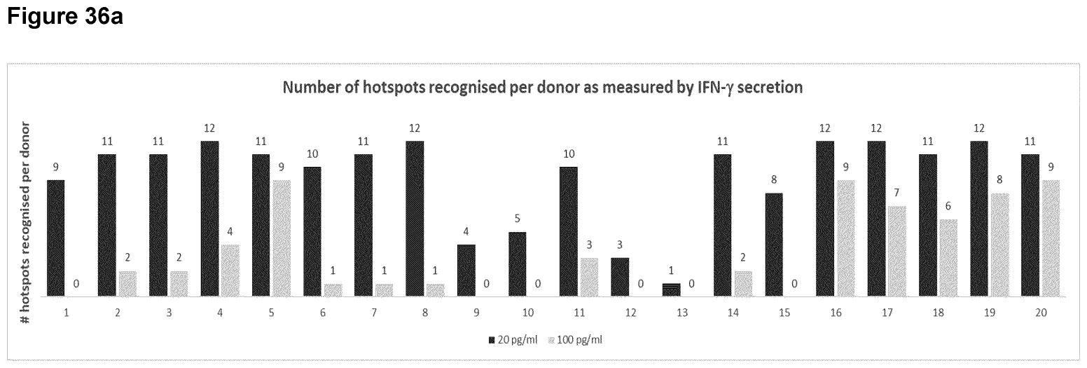

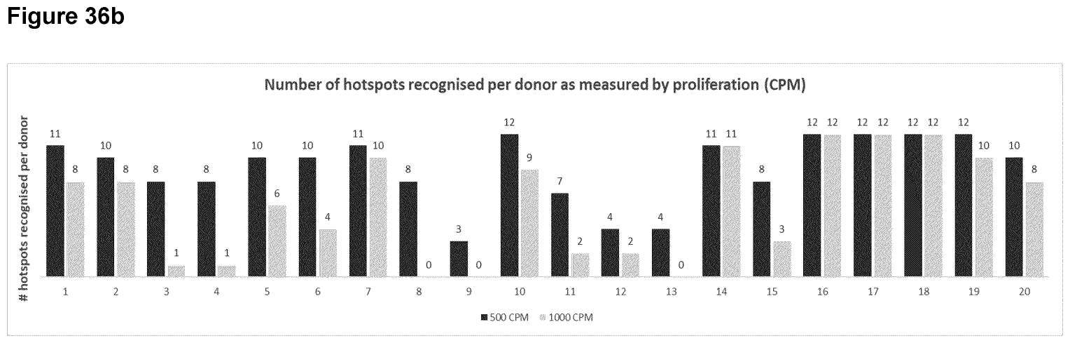

100% of the donors demonstrated antigen-specific T-cell responses against at least one epitope within one hotspot when using both IFN-γ secretion and T-cell proliferation readouts at the lower thresholds (20 pg/ml and 500 CPM respectively) as shown in FIGS. 36a & 36b below. PBMCs from 70% of donors demonstrated antigen-specific T-cell responses against peptides from at least 10/12 hotspots using the lower IFN-γ threshold (20 pg/ml) and 60% using the lower proliferation threshold (500 CPM). PBMCs from 75% of the donors demonstrated antigen-specific T-cell responses against at least one epitope within one hotspot when using the higher IFN-γ threshold (100 pg/ml) and 85% using the higher proliferation threshold (1000 CPM), and PBMCs from 90% of the donors had either a significant IFN-γ and/or a significant T cell proliferation response at the higher thresholds.

### 4. Discussion

SARS-CoV-2 hotspots identified in silico using the NEC Immune Profiler and subsequent Monte Carlo simulation analysis (shown in Table 2) were profiled to identify minimal epitopes for the most common HLA-A and HLA-B alleles in the Norwegian population. 65 test peptides (epitopes) were then synthesized and used to restimulate PBMCs from convalescent donors who had recovered from SARS-CoV-2 infection to assess whether the in silico predicted sequences could successfully induce T-cell recall responses. Demonstrating re-call responses in convalescent donors would provide compelling evidence that the predicted peptides and associated hotspots are capable of inducing antigen-specific T-cell responses during a natural infection, supporting their use for developing vaccines and diagnostics. Antigen-specific T-cell responses were measured using two readouts: IFN-γ secretion and T cell proliferation after restimulation with the test peptides.

100% of the tested peptides (epitopes), stimulated antigen-specific T-cell responses in the PBMCs from at least one donor when using the higher proliferation threshold of 1000 CPM, and 83% when using the higher IFN-γ threshold of 100 pg/ml. Similarly, 100% of the tested hotspots were shown to be immunogenic in at least 1 donor using both readouts at the higher thresholds. Interestingly, despite the lack of HLA-matching between the selected peptides and the donors (many who probably were not ethnic Norwegians), 100% of the donors demonstrated antigen-specific T-cell responses against at least one epitope when using both T cell readouts at the lower thresholds, and 90% of the donors had either a significant IFN-γ and/or a significant T cell proliferation response at the higher thresholds.

This data clearly supports the utility of using the hotspots, identified using the NEC Immune Profiler and subsequent Monte Carlo simulation analysis, as components of a universal T cell vaccine or diagnostic against SARS-CoV-2. Furthermore, since a vaccine incorporating the hotspots would contain multiple HLA-restricted T-cell epitopes that can be presented by a broad diversity of HLAs across the human population, it is likely to be more resistant to the emergence of escape variants than current generation of vaccines, that are designed to stimulate antibody responses against the Spike protein.

FIGS. 23-34 (i) below. The ELISA is capable of detecting IFN-γ concentrations of ≥10 pg/ml, but to be conservative, we have defined a positive response as being a test well that has an IFN-γ concentrations of ≥20 pg/ml (lower threshold). We have also applied a much more stringent threshold of ≥100 pg/ml to identify particularly strong responders (higher threshold).

FIGS. 23-34 (ii) below. We have defined a positive response as being a test well that has a CPM value above 500 (lower threshold) once the background CPM from the negative control has been subtracted. In addition, we have applied a more stringent threshold of ≥1000 CPM to identify particularly strong responders (higher threshold).

1. A coronavirus vaccine composition, comprising one or more epitopes found within any one or more hotspot regions identified in FIGS. 1-10, or a polynucleotide encoding said epitope,
wherein each epitope is at least 8 amino acids in length, and wherein each epitope has a mean antigen presentation (AP) cut off value according to the following table:

| | Mean Antigen |
|---|---|
| | Presentation |
| | (AP) |
| | Cut-Off |
| | Value |
| Averaged HLA Type of MHC Class I | ≥0.4 |
| Averaged HLA Type of MHC Class II | ≤13 |

or a mean immune presentation (IP) score of at least 0.5, and
wherein an antigen presentation (AP) value or immune presentation value is a prediction score assigned to each amino acid as shown in FIGS. 1-10, for each hotspot region,
and wherein the mean AP cut-off value is the value, averaged across all amino acids within an epitope, for which said epitope is considered able to stimulate a broad adaptive immune response across a plurality of HLA types, for either MHC Class I and/or MHC Class II immunogenicity.

2. The coronavirus vaccine composition according to claim 1, wherein each epitope has a mean antigen presentation (AP) cut off value according to the following table:

| | Mean Antigen |
|---|---|
| | Presentation |
| | (AP) |
| | Cut-Off |
| | Value |
| Averaged HLA Type of MHC Class I | ≥0.5 |
| Averaged HLA Type of MHC Class II | ≤10 |

3. A coronavirus vaccine composition, comprising an immunogenic portion of the coronavirus, said immunogenic portion consisting of one or more epitopes found within any one or more hotspot regions identified in FIGS. 1-10, or a polynucleotide encoding said epitope, wherein each of said epitope is at least 8 amino acids in length, and wherein each of said epitope is considered able to stimulate a broad adaptive immune response across a plurality of HLA types, for either MHC Class I and/or MHC Class II immunogenicity.

4. A coronavirus vaccine composition, comprising one or more epitopes found within Table 1, or a polynucleotide encoding said epitope, wherein each epitope is at least 8 amino acids in length, preferably 9 amino acids, and wherein the epitope is considered able to stimulate a broad adaptive immune response across a plurality of HLA types, for MHC Class I immunogenicity,
optionally wherein said composition also further comprises any of the one or more epitopes according to claim 1.

5. The coronavirus vaccine composition according to claim 1, wherein the one or more epitopes are found within any one or more of FIGS. 13-14.

6. The coronavirus vaccine composition according to claim 1, wherein the one or more epitopes are found within any one or more of FIGS. 15-16

7. The coronavirus vaccine composition according to claim 1, wherein the one or more epitopes are found within any one or more of FIGS. 17-18.

8. The coronavirus vaccine composition according to claim 1, wherein said composition comprises at least 5 epitopes.

9. The coronavirus vaccine composition according to claim 1, wherein said composition comprises between 5 and 10 epitopes.

10. The coronavirus vaccine composition according to claim 1, wherein said composition comprises between 5 and 20 epitopes.

11. The coronavirus vaccine composition according to claim 1, wherein said composition comprises at least one epitope that is considered able to stimulate a broad adaptive immune response across a plurality of HLA types for MHC Class I,
and at least one epitope that is considered able to stimulate a broad adaptive immune response across a plurality of HLA types for MHC Class II.

12. The coronavirus vaccine composition according to claim 1, wherein each epitope has a maximum length of 25 amino acids.

13. The coronavirus vaccine composition according to claim 1, wherein the composition comprises one or more discrete hotspot regions identified in any of FIGS. 13 to 18, or a portion thereof such that said portion comprises at least one epitope as defined herein.

14. The coronavirus vaccine composition according to claim 13, wherein the one or more discrete hotspot regions, or the portion thereof, are identified in FIG. 15 or FIG. 16.

15. The coronavirus vaccine composition according to claim 13, wherein the one or more discrete hotspot regions, or the portion thereof, are identified in FIG. 17 or FIG. 18.

16. The coronavirus composition according to claim 13, wherein the discrete hotspot regions, or the portion thereof, are comprised within an expression cassette.

17. The coronavirus composition according to claim 1, wherein the epitopes or hotspot regions in the composition are in the form of DNA or RNA sequences.

18. The coronavirus composition according to claim 1, wherein the epitope(s) or hotspot region(s) are in the composition in the form of peptides.

19. The coronavirus vaccine composition according to claim 1, wherein said one or more epitopes are comprised within a cassette.

20. The coronavirus vaccine composition according to claim 1, further comprising full recombinant SARS-CoV-2 spike(S) protein or one or more domains thereof.

21. The coronavirus vaccine composition according to claim 1, further comprising a pharmaceutically acceptable carrier, diluent, excipient and/or adjuvant.

22. A coronavirus vaccine composition according to claim 1, for use in the therapeutic or prophylactic treatment of a coronavirus infection in a subject.

23. The coronavirus vaccine composition for use according to claim 22, wherein the coronavirus infection is caused by SARS-CoV-2, SARS-CoV, or MERS-CoV.

24. The coronavirus vaccine composition for use according to claim 22, wherein the coronavirus infection is caused by SARS-CoV-2.

25. The coronavirus vaccine composition for use according to claim 22, wherein said composition is administered to the subject via a parental, oral, sublingual, nasal, naso-oral, or pulmonary route.

26. The coronavirus vaccine composition for use according to claim 25, wherein said parental route is a subcutaneous, intradermal, intramuscular, subdermal, intraperitoneal, or intravenous injection.

27. The coronavirus vaccine composition for use according to claim 25, wherein said composition is administered to the subject via one or more intradermal infections.

28. The use of a coronavirus vaccine composition according to claim 1, in the manufacture of a medicament for the therapeutic or prophylactic treatment of a coronavirus infection.

29. A diagnostic assay to determine whether a patient has or has had prior infection with SARS-CoV-2, wherein the diagnostic assay is carried out on a biological sample obtained from a subject, and wherein the diagnostic assay comprises the utilisation or identification within the biological sample of one or more epitopes according to claim 1.

30. The diagnostic assay according to claim 29, wherein the assay is an enzyme-linked immune absorbent spot (ELISPOT) assay, enzyme-linked immunosorbent assay (ELISA), cytokine capture assay, intracellular staining assay, tetramer staining assay, or a limiting dilution culture assay.

31. The diagnostic assay according to claim 29, wherein said diagnostic assay comprises identification of an immune system component within the biological sample that recognises said one or more epitopes.
